SEQUENCE LISTING (166 sequences)

SEQ ID NO: 1 - DNA, 9171 bp, synthetic construct
  source 1..9171: mol_type = other DNA
ggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcggctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggaagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccacgcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggtagggataacagggtaattatagggcgaattcgagctcggtacccggctcgagtctagagtcgacctgcaggcatgcaattaccctgttatccctactcactcaaaggcggtaatacggtt

SEQ ID NO: 2 - DNA, 8582 bp, synthetic construct
  source 1..8582: mol_type = other DNA
ttttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtctcgcgcgtttcggtgatgacggtggtatcgtttgaacacggcattagtcagggaagtcataacacagtcctttcccgcaattttctttttctattactcttggcctcctctagtacactctatatttttttatgcctcggtaatgattttcatttttttttttcccctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctactggcgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttcctttaccgcatcaggaaattgtaagtgtgaaataccgcacagatgcgtaaggtagggataacagggtaattatagggcgaattctattttacgcgcaagccggacccgcgcttgcacggctgaggtgcgtagggaacgtcgcaaagaggcatgaatattcagcagcacagaacaaattaatcacttaattaaatcgtcgatggatccacttgatacctagttaacccacgggtgtggaaaagctccctaagggaggaacggacgccggaaggttttcctgagtggcactcacacattatggtgtcaggcatgcattaccctgttatccctactcactcaaaggcggtaatacggttggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcggctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcga

SEQ ID NO: 3 - DNA, 3422 bp, synthetic construct
  source 1..3422: mol_type = other DNA
tctaaactagaaagttaccgttagtcttactgatccgaaccgtcattagttactagaggaaacgtgtcaagaatgccagccgatcccacattcttaattactcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccatgacgagtacggtgggtaggccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaactgcttggcaacctaatgctannannannannannannannannannannataccgttcgtataatgtgtactatacgaagttattcaatcggtgtcacttatggataacttcgtataatgtatgctatacgaacggtaacttgatacctagttaacccacgggtgtggaaaagctccctaagggaggaacggacgccggaaggttttcctgagtggcactcacacattatggtgtcag

SEQ ID NO: 4 - DNA, 515 bp, synthetic construct
  source 1..515: mol_type = other DNA
tattttacgcgcaagccggacccgcgcttgcacggctgaggtgcgtagggaacgtcgcaaagaggcatgaatattcagcagcacagaacaaattaatcacgagccaatgagattttaaagggtatattacttatcttatcgataagcagtattgatattaaagggacagttttatcgttggttaatatggaaaaagtgatgaccatgatgcctttcttaaaaagagtatttctttttatttcactttcacctcgagagtcctaggtatttttaggaaattgatgataccgtgggaataatgagagataacataaataaagtagcagaaagaggtgaaagattaacgtccattgaagataaagccgataacctagcggtctcagcccaaggctttaagaggggtgccaatagggtccttacatatcctctacaccgagcgcgtcgacccgtcgaatgaatgtgagttgcgtgttgttccttcggaaatgttgctaaaaaacagagccattcctttt

SEQ ID NO: 5 - DNA, 514 bp, synthetic construct
  source 1..514: mol_type = other DNA
cttacatatcctctacaccgagcgcgtcgacccgtcgaatgaatgtgagttgcgtgttgttccttcggaaatgttgctaaaaaacagagccattcctttttatatgtatatatacatatatacatatatgggcgtatatttactttgttcttattttctgtctaattttataattttactgacagtagctaagccctctgtattgctgttctgtgttattgcactagtgtcataacgcagatggtttttactcgagagtcctagctatttgtacgagttcgtcaggataaatcgtcagtaccatttttcttgtggctagttggcttcaaccaaacgtcctcttctctcttatggcaagaagaaagttatatgtgtgactggttgtttatttcactttcgcgactgaaagcgccgtctaaactagaaagttaccgttagtcttactgatccgaaccgtcattagttactagaggaaacgtgtcaagaatgccagccgatcccacattcttaatta

SEQ ID NO: 6 - DNA, 515 bp, synthetic construct
  source 1..515: mol_type = other DNA
aatcaaagggaagtcgaataaaggtcatccagggaattagccgttttcgaccaagtctttacgggagtagaagacgaatcctctcgtgtgctggcccaggaaattaagatagttattcgagtgattgccaaatatcatgttctacttcgaagacttatagctaattaattttttcataatgaaggtgtcgttaattgttctgattagtaacatgaaactcaaaaatcatcaaaaaaagaaaagctaaatgctcgagagtcctaggaaaggatcatggctggcagcgcccacaacaattaaactctgttactatcaaaaaacattgagcccaagaatggaataaaattttcactacacctcggacatggatttgtacatgtcctattatcctgtaattttgacatatactgatatgacttgatacctagttaacccacgggtgtggaaaagctccctaagggaggaacggacgccggaaggttttcctgagtggcactcacacattatggtgtcag

SEQ ID NO: 7 - DNA, 455 bp, synthetic construct
  source 1..455: mol_type = other DNA
tattttacgcgcaagccggacccgcgcttgcacggctgaggtgcgtagggaacgtcgcaaagaggcatgaatattcagcagcacagaacaaattaatcactagaaacggctgacaaccattttgttagtcctcggaaaatctccgaacaaatgagaaagcaaaatataataacacagagtttggcgagaagaagatatatgataactactattaaggaccctcgagagtcctaggtagaaacggctgacaaccattttgttagtcctcggaaaatctccgaacaaatgagaaagcaaaatataataacacagagtttggcgagaagaagatatatgataactactattaaggacccttacatatcctctacaccgagcgcgtcgacccgtcgaatgaatgtgagttgcgtgttgttccttcggaaatgttgctaaaaaacagagccattcctttt

SEQ ID NO: 8 - DNA, 455 bp, synthetic construct
  source 1..455: mol_type = other DNA
cttacatatcctctacaccgagcgcgtcgacccgtcgaatgaatgtgagttgcgtgttgttccttcggaaatgttgctaaaaaacagagccattcctttttatatatgagagcgtatatgtactttgttgtttttattttttcctgcttagttctataatcttattgacatttgtcaagccctctgtattgcagttatgtggtattgcaccggtgtcatactcgagagtcctaggtcacatgttacttttataaaataattatattatattttaaatatatcaaagtttctttcattttttagattactttaagataataaatatatgtttttggtttccgaaacgtttaaaatatctaaactagaaagttaccgttagtcttactgatccgaaccgtcattagttactagaggaaacgtgtcaagaatgccagccgatcccacattcttaatta

SEQ ID NO: 9 - DNA, 455 bp, synthetic construct
  source 1..455: mol_type = other DNA
aatcaaagggaagtcgaataaaggtcatccagggaattagccgttttcgaccaagtctttacgggagtagaagacgaatcctctcgtgtgctggcccaggccagcgatagcccattgtgtaatgtgtatttctaaccatcaaacactatacctctacgccaaatagacaacgcaagacactctataaacggaaacccaatgcaccataaatcattaaggactcgagagtcctaggccagcgatagcccattgtgtaatgtgtatttctaaccatcaaacactatacctctacgccaaatagacaacgcaagacactctataaacggaaacccaatgcaccataaatcattaaggaacttgatacctagttaacccacgggtgtggaaaagctccctaagggaggaacggacgccggaaggttttcctgagtggcactcacacattatggtgtcag

SEQ ID NO: 10 - DNA, 20 bp, synthetic construct
  source 1..20: mol_type = other DNA
cattaaaaagaaggcatagg

SEQ ID NO: 11 - DNA, 20 bp, synthetic construct
  source 1..20: mol_type = other DNA
tttgaagaaaggaaaaaatg

SEQ ID NO: 12 - DNA, 20 bp, synthetic construct
  source 1..20: mol_type = other DNA
ggccaatcaagcaaactgaa

SEQ ID NO: 13 - DNA, 20 bp, synthetic construct
  source 1..20: mol_type = other DNA
tctaaaaatgaagatgtgtc

SEQ ID NO: 14 - DNA, 20 bp, synthetic construct
  source 1..20: mol_type = other DNA
acacagaacagcaatacaga

SEQ ID NO: 15 - DNA, 20 bp, synthetic construct
  source 1..20: mol_type = other DNA
aacacagaacagcaatacag

SEQ ID NO: 16 - DNA, 20 bp, synthetic construct
  source 1..20: mol_type = other DNA
ttttagatccttgtaccaca

SEQ ID NO: 17 - DNA, 20 bp, synthetic construct
  source 1..20: mol_type = other DNA
tctaaaaatgaagatgtgtc

SEQ ID NO: 18 - DNA, 20 bp, synthetic construct
  source 1..20: mol_type = other DNA
ctgcattttcaaataccgct

SEQ ID NO: 19 - DNA, 20 bp, synthetic construct
  source 1..20: mol_type = other DNA
gttttttgcatcataaaggg

SEQ ID NO: 20 - DNA, 20 bp, synthetic construct
  source 1..20: mol_type = other DNA
tcaatataataagcatacta

SEQ ID NO: 21 - DNA, 20 bp, synthetic construct
  source 1..20: mol_type = other DNA
cacctttcgtctgctgcctc

SEQ ID NO: 22 - DNA, 20 bp, synthetic construct
  source 1..20: mol_type = other DNA
cacccgaggcagcagacgaa

SEQ ID NO: 23 - DNA, 20 bp, synthetic construct
  source 1..20: mol_type = other DNA
ataatttgaaaaaacacccg

SEQ ID NO: 24 - DNA, 20 bp, synthetic construct
  source 1..20: mol_type = other DNA
atcctttttttatttgaaag

SEQ ID NO: 25 - DNA, 20 bp, synthetic construct
  source 1..20: mol_type = other DNA
aataaaaaaaggatcatggc

SEQ ID NO: 26 - DNA, 20 bp, synthetic construct
  source 1..20: mol_type = other DNA
aaaaacattgagcccaagaa

SEQ ID NO: 27 - DNA, 20 bp, synthetic construct
  source 1..20: mol_type = other DNA
cattataaaaattaaaaccg

SEQ ID NO: 28 - DNA, 20 bp, synthetic construct
  source 1..20: mol_type = other DNA
gattaacggagctgcccaag

SEQ ID NO: 29 - DNA, 20 bp, synthetic construct
  source 1..20: mol_type = other DNA
atggaaacctccttctgtgg

SEQ ID NO: 30 - DNA, 20 bp, synthetic construct
  source 1..20: mol_type = other DNA
caatgtttaactaatactct

SEQ ID NO: 31 - DNA, 20 bp, synthetic construct
  source 1..20: mol_type = other DNA
tctctgctcaaggtttcaag

SEQ ID NO: 32 - DNA, 20 bp, synthetic construct
  source 1..20: mol_type = other DNA
atagggtgaggaaagccatg

SEQ ID NO: 33 - DNA, 20 bp, synthetic construct
  source 1..20: mol_type = other DNA
ttagtcgatagaaattcact

SEQ ID NO: 34 - DNA, 20 bp, synthetic construct
  source 1..20: mol_type = other DNA
gaatactatatatatatagg

SEQ ID NO: 35 - DNA, 20 bp, synthetic construct
  source 1..20: mol_type = other DNA
ataagattatagaactaagc

SEQ ID NO: 36 - DNA, 20 bp, synthetic construct
  source 1..20: mol_type = other DNA
atacccgtccagaatgtgag

SEQ ID NO: 37 - DNA, 20 bp, synthetic construct
  source 1..20: mol_type = other DNA
tacccgtccagaatgtgaga

SEQ ID NO: 38 - DNA, 20 bp, synthetic construct
  source 1..20: mol_type = other DNA
atagattttctgaccagtag

SEQ ID NO: 39 - DNA, 20 bp, synthetic construct
  source 1..20: mol_type = other DNA
cataactaactcgattcgca

SEQ ID NO: 40 - DNA, 20 bp, synthetic construct
  source 1..20: mol_type = other DNA
atggattgccaacgatgatg

SEQ ID NO: 41 - DNA, 20 bp, synthetic construct
  source 1..20: mol_type = other DNA
actgagagccaagaattcaa

SEQ ID NO: 42 - DNA, 20 bp, synthetic construct
  source 1..20: mol_type = other DNA
aaagttgagatattctggat

SEQ ID NO: 43 - DNA, 20 bp, synthetic construct
  source 1..20: mol_type = other DNA
caaacttcatgcaggatgct

SEQ ID NO: 44 - DNA, 20 bp, synthetic construct
  source 1..20: mol_type = other DNA
aatttcggaaaaaaagtccc

SEQ ID NO: 45 - DNA, 18 bp, synthetic construct
  source 1..18: mol_type = other DNA
ggaagcgtaggccaccct

SEQ ID NO: 46 - DNA, 21 bp, synthetic construct
  source 1..21: mol_type = other DNA
tccgagcacgatgaagaacga

SEQ ID NO: 47 - DNA, 20 bp, synthetic construct
  source 1..20: mol_type = other DNA
aagcctgctaacaagggcga

SEQ ID NO: 48 - DNA, 20 bp, synthetic construct
  source 1..20: mol_type = other DNA
ccagttacctgcgccaaatc

SEQ ID NO: 49 - DNA, 20 bp, synthetic construct
  source 1..20: mol_type = other DNA
ctggcttcacccactgcaga

SEQ ID NO: 50 - DNA, 19 bp, synthetic construct
  source 1..19: mol_type = other DNA
ccatgagctgcgtggcatt

SEQ ID NO: 51 - DNA, 20 bp, synthetic construct
  source 1..20: mol_type = other DNA
ccgatgggtgaaagcgtagc

SEQ ID NO: 52 - DNA, 21 bp, synthetic construct
  source 1..21: mol_type = other DNA
gaacggcctcagaatgtgcag

SEQ ID NO: 53 - DNA, 20 bp, synthetic construct
  source 1..20: mol_type = other DNA
gaggcgaagctggagtaccg

SEQ ID NO: 54 - DNA, 19 bp, synthetic construct
  source 1..19: mol_type = other DNA
aagcgccccttctgatgca

SEQ ID NO: 55 - DNA, 21 bp, synthetic construct
  source 1..21: mol_type = other DNA
aacagtgacaccacaccctct

SEQ ID NO: 56 - DNA, 20 bp, synthetic construct
  source 1..20: mol_type = other DNA
cttgttggcgtgtgaggagg

SEQ ID NO: 57 - DNA, 22 bp, synthetic construct
  source 1..22: mol_type = other DNA
aaagttgcaggaccacttctgc

SEQ ID NO: 58 - DNA, 20 bp, synthetic construct
  source 1..20: mol_type = other DNA
cccagatgcgaagttaagtg

SEQ ID NO: 59 - DNA, 21 bp, synthetic construct
  source 1..21: mol_type = other DNA
caagggagacgcattgggtca

SEQ ID NO: 60 - DNA, 19 bp, synthetic construct
  source 1..19: mol_type = other DNA
gcctggttgctacgcctga

SEQ ID NO: 61 - DNA, 482 bp, synthetic construct
  source 1..482: mol_type = other DNA
gaattctttccgaatgacatgcgtctccttgcgggtaaatcaccgaccgcaattcatagaagcctgggggaacagataggtctaattagcttaagagagtaaatcctcatatgtacatttatcgagccaatcgagggcagcagtttaacatcaagccggatttgctcacgctactttgaccccttttcgtttcgacggagagaagaaaccggtgttttcctatccttgcctctcgaggcgcgcatatcctaggtggtttcgatagaggccaagcgaataatactccttttccaggttattttggaggtttggatttatttgattatgactttttgtttggcaatgactttgcttaaaaattttctttccaaactccagccgcgctatccggtaatctccaaattaaaacataccgttccatgaaggctagaattacttaccggccttttccatgcctgcgctatacccccccactcgcatgc

SEQ ID NO: 62 - DNA, 483 bp, synthetic construct
  source 1..483: mol_type = other DNA
gaattcagccgcgctatccggtaatctccaaattaaaacataccgttccatgaaggctagaattacttaccggccttttccatgcctgcgctatacccccccactcaaatgcaacacttccctaatatagaaatttgggcattaattattttgagaattttgatgatttgaataatttcattaacgtaaaggaacatagtgctacgaatccaacagtggacccaaaaatgactcgaggcgcgcatatcctaggatgcctgccactaggaatccatcgacgtaccatggctataactttccttatgttgtttgcttagttttttgatattagtgttgcttatgtgaaatttcgcgatttcaattaaaataataaatacatatgcatatggtccacaggacactcgtcgctttcggatttgccctctatgtggcggttttcaggcacacttatgctcagcaccgtttaaaccagaccgacgcatg

SEQ ID NO: 63 - DNA, 483 bp, synthetic construct
  source 1..483: mol_type = other DNA
gaattctatgcatatggtccacaggacactcgtcgctttcggatttgccctctatgtggcggttttcaggcacacttatgctcagcaccgtttaaaccagaccgacctttccagattacaagaactttgcgtttggcctctacaacgattctcacaagcataagggccatgctggtgtacagggaaatgtctctgctgagacacatttccggattgagatggtcagtaaaactcgaggcgcgcatatcctaggttggtggtttaatctattttttataaaaaatgacgcgggcagattcaattagtgtcctaaatttattcgcgtttcaagatttcaaaggattgatcctcttatcagaaacgataagtgctactccgaactacgatgccgctaagaacctctcggtcgtcgctgacgtttacactctagtctcattataatcgttcgctattcagggattgaccaacaccggaaaacgcatg

SEQ ID NO: 64 - DNA, 484 bp, synthetic construct
  source 1..484: mol_type = other DNA
gaattcaactacgatgccgctaagaacctctcggtcgtcgctgacgtttacactctagtctcattataatcgttcgctattcagggattgaccaacaccggaaaacttttttcatttcttatattattttttgttcgagaatcactttttcaagatggtaacaacatcttcgtcttccaaaatgtgactcaaccccacgtattgaggttgatgtttgacactgctaccgtactcgaggcgcgcatatcctaggtggtacaaggatctaaaaatgaagatgtgtctggctttagtaatcatcatattgcttgttgtaatcatcgtccccattgctgttcactttagtcgatagaagttcactcgcaatgctatatatatgtcactagaggacgcacgctctatttttatgatccattgatgtccctgacgctgcaaaatttgcaaccaggcagtcttcgcggtaggtcctagtgcaatggcatgc

SEQ ID NO: 65 - DNA, 484 bp, synthetic construct
  source 1..484: mol_type = other DNA
gaattcgtcactagaggacgcacgctctatttttatgatccattgatgtccctgacgctgcaaaatttgcaaccaggcagtcttcgcggtaggtcctagtgcaatgaggaattgagtatgcagaatcagaataaaggctgactttcaaaaaaaggttgtattacaattgcaggttttcgataaaagagaccctattctcatctactactgctaaacttcgagatattttcgctcgaggcgcgcatatcctaggctgttgctgttgctgctgcgggcccacattagggtagcctagtaaagaagggtcgttcattagcggtagtttgcagggataacgtcagtcggagttcccttgctggtgtcccttatgctgtgcccatactcttccataaacgggctattagttatgaggtccgaagattgaaaaaggtgagggaactcggccgaacgggaaagacggacatctaggcaacctgacgcatgc

SEQ ID NO: 66 - DNA, 484 bp, synthetic construct
  source 1..484: mol_type = other DNA
gaattcatactcttccataaacgggctattagttatgaggtccgaagattgaaaaaggtgagggaactcggccgaacgggaaagacggacatctaggcaacctgactggcgtgtgctatagtgctctatattcgagtttgttgctactggtggacacccgactatctacagtaaggaacgtaaacaagaaaaagagagaaaatacgctatagttgaaaacatgagtggttcctcgaggcgcgcatatcctaggttgtttgattttggtccttttctccggtaggagttctgattctggcccagtttcagtctttaccagcggtcttttcctcagaattgccatagatgagtatttactgatcttttgcatatttttttgtcgctcttgggccgcggtgcgctaccttgcaggaattgagaccgtccgttaatttcccttgcatatatattgcgtttctttgaccttttaaccgctctcgcatgc

SEQ ID NO: 67 - DNA, 484 bp, synthetic construct
  source 1..484: mol_type = other DNA
gaattgtcgctcttgggccgcggtgcgctaccttgcaggaattgagaccgtccgttaatttcccttgcatatatattgcgtttctttgaccttttaaccgctctccggcaaagtacaagggaaggaagcacagaagcaagaggaggcgcatcgatcgtggcagatgagtcagcaaacaccacaggaaagtgaacagaccacagcgaaagaacaggaccttgatcaagagagctcgaggcgcgcatatcctaggactcataatatcttgtcgaaaatacgcggtgtagggagttatggtggataactttttcacgattagaagaaaaggaaaatttcattattcgtagcttaacatggcaaaaacgagaaagacatatacaaaagcttaaatgggaaatacgcgcccataacttggtgcgaatacgggtcgtagcaatgttcgtctgactatgatctacatattacaggcggtacgtctgcatgc

SEQ ID NO: 68 - DNA, 484 bp, synthetic construct
  source 1..484: mol_type = other DNA
gaattccaaaagcttaaatgggaaatacgcgcccataacttggtgcgaatacgggtcgtagcaatgttcgtctgactatgatctacatattacaggcggtacgtctcatgaagcgaaagtacttcacgacacctagattgcaatctactcaatgttatccctggatgaaatattatttcgttaacgaccatagtaactacctgcttccatatgtttggcctaatggaaccactcgaggcgcgcatatcctaggaatcgtcagtaccatttttcttgtggctagttggcttcaaccaaacgtcctcttctctcttatggcaagaagaaagttatatgtgtgactggttgtttatttcactttcgcgactgaaagcgccgtctaaactagaaagttaccgttagtcttactgatccgaaccgtcattagttactagaggaaacgtgtcaagaatgccagccgatcccacattcttaattagcatgc

SEQ ID NO: 69 - DNA, 484 bp, synthetic construct
  source 1..484: mol_type = other DNA
gaattcaatcaaagggaagtcgaataaaggtcatccagggaattagccgttttcgaccaagtctttacgggagtagaagacgaatcctctcgtgtgctggcccaggaaattaagatagttattcgagtgattgccaaatatcatgttctacttcgaagacttatagctaattaattttttcataatgaaggtgtcgttaattgttctgattagtaacatgaaactcaaaaactcgaggcgcgcatatcctaggagtagttgtttatcactagacatataattatgtttatttatatttagtgggagcaaaacagtttattgaatgtttaccagaaccgaaaaaaaagctcttctaaactgttgacatccagttcattttactaggtaactagaggatttgcgacgttctaagcgttggtccatgtgaatcgccatccaggatcacgtcgccctgaaaaaaagatatcagcaactctccgcatgc

SEQ ID NO: 70 - DNA, 484 bp, synthetic construct
  source 1..484: mol_type = other DNA
gaattctactaggtaactagaggatttgcgacgttctaagcgttggtccatgtgaatcgccatccaggatcacgtcgccctgaaaaaaagatatcagcaactctcctacccgtatgtattaatgtataaatgttctcagagcaaattttatcgatatcttgtttgccagtggtatgcaggtttggcaaattttttaccataatatccgtttatagattctggaaccttaccctcgaggcgcgcatatcctaggcctgaaaatttgaggttgttcacggaaatcatttggttatgtctgtcggcctgctatttagagacattttttattgcaacaacctactctatgcacttacacggaatcgcagaataacgcgcccatgctctaggcatctaactattcccactgccttaagggggcctgcgttttctgcctgtcgatccataggactcgtgtcaacgcgcaggcttagttcgagatgcatgc

SEQ ID NO: 71 - DNA, 484 bp, synthetic construct
  source 1..484: mol_type = other DNA
gaattctgctctaggcatctaactattcccactgccttaagggggcctgcgttttctgcctgtcgatccataggactcgtgtcaacgcgcaggcttagttcgagatgggtgtaaaagttatgtacgctcgaaacaaattttatgtagtttactttagatgcaaatgctattatatattttgctttatgatcctcggcttgatgctcgccaacgtgagatagctggtcatcactcgaggcgcgcatatcctagggacaataactggcaagggctctcactaaatatcaacccctttcaaataaaaaaaggatcatggctggcagcgcccacaacaattaaactctgttactatcaaaaaacattgagcccaagaatggaacttgatacctagttaacccacgggtgtggaaaagctccctaagggaggaacggacgccggaaggttttcctgagtggcactcacacattatggtgtcaggcatgc

SEQ ID NO: 72 - DNA, 20 bp, synthetic construct
  source 1..20: mol_type = other DNA
aattgatgaaatgaataggt

SEQ ID NO: 73 - DNA, 20 bp, synthetic construct
  source 1..20: mol_type = other DNA
atatatatatatgtatataa

SEQ ID NO: 74 - DNA, 20 bp, synthetic construct
  source 1..20: mol_type = other DNA
acgttctcgtgttaatcccg

SEQ ID NO: 75 - DNA, 20 bp, synthetic construct
  source 1..20: mol_type = other DNA
ttagtaaaacaagaagaccg

SEQ ID NO: 76 - DNA, 20 bp, synthetic construct
  source 1..20: mol_type = other DNA
caacacaattgggaaacgat

SEQ ID NO: 77 - DNA, 20 bp, synthetic construct
  source 1..20: mol_type = other DNA
aagcatactcatcttcaact

SEQ ID NO: 78 - DNA, 20 bp, synthetic construct
  source 1..20: mol_type = other DNA
tattcagtgaggagttacac

SEQ ID NO: 79 - DNA, 20 bp, synthetic construct
  source 1..20: mol_type = other DNA
tgtatgaatcatattcagtg

SEQ ID NO: 80 - DNA, 20 bp, synthetic construct
  source 1..20: mol_type = other DNA
gatggactgacacttaattg

SEQ ID NO: 81 - DNA, 20 bp, synthetic construct
  source 1..20: mol_type = other DNA
aattaagtgtcagtccatct

SEQ ID NO: 82 - DNA, 20 bp, synthetic construct
  source 1..20: mol_type = other DNA
ataaaattttcactacacct

SEQ ID NO: 83 - DNA, 20 bp, synthetic construct
  source 1..20: mol_type = other DNA
catgtacaaatccatgtccg

SEQ ID NO: 84 - DNA, 20 bp, synthetic construct
  source 1..20: mol_type = other DNA
tatataaagaatatacacag

SEQ ID NO: 85 - DNA, 20 bp, synthetic construct
  source 1..20: mol_type = other DNA
atataaagaatatacacaga

SEQ ID NO: 86 - DNA, 20 bp, synthetic construct
  source 1..20: mol_type = other DNA
tctgctgtagttagacgtag

SEQ ID NO: 87 - DNA, 20 bp, synthetic construct
  source 1..20: mol_type = other DNA
ctatgatatctggcctaagg

SEQ ID NO: 88 - DNA, 20 bp, synthetic construct
  source 1..20: mol_type = other DNA
agatggctagaagaatagga

SEQ ID NO: 89 - DNA, 20 bp, synthetic construct
  source 1..20: mol_type = other DNA
agatgggaagaaaactgaag

SEQ ID NO: 90 - DNA, 20 bp, synthetic construct
  source 1..20: mol_type = other DNA
atcctgaagagactggaatg

SEQ ID NO: 91 - DNA, 20 bp, synthetic construct
  source 1..20: mol_type = other DNA
gcattccagtctcttcagga

SEQ ID NO: 92 - DNA, 20 bp, synthetic construct
  source 1..20: mol_type = other DNA
ggagcttccatatgtagtgt

SEQ ID NO: 93 - DNA, 20 bp, synthetic construct
  source 1..20: mol_type = other DNA
catatatcctacactacata

SEQ ID NO: 94 - DNA, 20 bp, synthetic construct
  source 1..20: mol_type = other DNA
agcatttgtattcgaaaagg

SEQ ID NO: 95 - DNA, 20 bp, synthetic construct
  source 1..20: mol_type = other DNA
aagcatttgtattcgaaaag

SEQ ID NO: 96 - DNA, 20 bp, synthetic construct
  source 1..20: mol_type = other DNA
gagaaggttctcaagcacaa

SEQ ID NO: 97 - DNA, 20 bp, synthetic construct
  source 1..20: mol_type = other DNA
cgatgagaattaatgctgaa

SEQ ID NO: 98 - DNA, 20 bp, synthetic construct
  source 1..20: mol_type = other DNA
aaaaaaggtgataactccgt

SEQ ID NO: 99 - DNA, 20 bp, synthetic construct
  source 1..20: mol_type = other DNA
tgataactccgtaggaattg

SEQ ID NO: 100 - DNA, 20 bp, synthetic construct
  source 1..20: mol_type = other DNA
tatatgatgaatcattaaag

SEQ ID NO: 101 - DNA, 20 bp, synthetic construct
  source 1..20: mol_type = other DNA
atgatgaatcattaaagagg

SEQ ID NO: 102 - DNA, 20 bp, synthetic construct
  source 1..20: mol_type = other DNA
cagatccaaagcgcaagtga

SEQ ID NO: 103 - DNA, 20 bp, synthetic construct
  source 1..20: mol_type = other DNA
cacgtaacttaaatcagtac

SEQ ID NO: 104 - DNA, 20 bp, synthetic construct
  source 1..20: mol_type = other DNA
atcaaaacgtgagtttcctg

SEQ ID NO: 105 - DNA, 20 bp, synthetic construct
  source 1..20: mol_type = other DNA
aggtatatcatcgtaaccag

SEQ ID NO: 106 - DNA, 20 bp, synthetic construct
  source 1..20: mol_type = other DNA
cacgactttgatcacccgaa

SEQ ID NO: 107 - DNA, 20 bp, synthetic construct
  source 1..20: mol_type = other DNA
ataaactcataacagtgcaa

SEQ ID NO: 108 - DNA, 20 bp, synthetic construct
  source 1..20: mol_type = other DNA
ttatgatgcaaaaaaccaag

SEQ ID NO: 109 - DNA, 20 bp, synthetic construct
  source 1..20: mol_type = other DNA
ctgcattttcaaataccgct

SEQ ID NO: 110 - DNA, 20 bp, synthetic construct
  source 1..20: mol_type = other DNA
tgaatctacataatgaaatg

SEQ ID NO: 111 - DNA, 20 bp, synthetic construct
  source 1..20: mol_type = other DNA
aaatatcagaaatatgcaaa

SEQ ID NO: 112 - DNA, 20 bp, synthetic construct
  source 1..20: mol_type = other DNA
acttccacgtgtagatgtga

SEQ ID NO: 113 - DNA, 20 bp, synthetic construct
  source 1..20: mol_type = other DNA
tgttccttcacatctacacg

SEQ ID NO: 114 - DNA, 20 bp, synthetic construct
  source 1..20: mol_type = other DNA
cacctttcgtctgctgcctc

SEQ ID NO: 115 - DNA, 20 bp, synthetic construct
  source 1..20: mol_type = other DNA
ataatttgaaaaaacacccg

SEQ ID NO: 116 - DNA, 18 bp, synthetic construct
  source 1..18: mol_type = other DNA
ggaagcgtaggccaccct

SEQ ID NO: 117 - DNA, 20 bp, synthetic construct
  source 1..20: mol_type = other DNA
gagccacaaatcccacacca

SEQ ID NO: 118 - DNA, 22 bp, synthetic construct
  source 1..22: mol_type = other DNA
agagcttggactacaacagtgg

SEQ ID NO: 119 - DNA, 20 bp, synthetic construct
  source 1..20: mol_type = other DNA
gagatgtagagcctgctgca

SEQ ID NO: 120 - DNA, 22 bp, synthetic construct
  source 1..22: mol_type = other DNA
acccactgcacatatcatccag

SEQ ID NO: 121 - DNA, 20 bp, synthetic construct
  source 1..20: mol_type = other DNA
atcagtgtcattgcccagcg

SEQ ID NO: 122 - DNA, 20 bp, synthetic construct
  source 1..20: mol_type = other DNA
tctcatcaagcagcaccagc

SEQ ID NO: 123 - DNA, 20 bp, synthetic construct
  source 1..20: mol_type = other DNA
tggtgagggcgttaatgtgg

SEQ ID NO: 124 - DNA, 21 bp, synthetic construct
  source 1..21: mol_type = other DNA
ggactgatgatgttgaagccc

SEQ ID NO: 125 - DNA, 20 bp, synthetic construct
  source 1..20: mol_type = other DNA
tgctgctgtagttggtgagc

SEQ ID NO: 126 - DNA, 21 bp, synthetic construct
  source 1..21: mol_type = other DNA
tgccgtgcctatttcgtctgg

SEQ ID NO: 127 - DNA, 21 bp, synthetic construct
  source 1..21: mol_type = other DNA
ggactgcggaactacaagctg

SEQ ID NO: 128 - DNA, 20 bp, synthetic construct
  source 1..20: mol_type = other DNA
aagcctgctaacaagggcga

SEQ ID NO: 129 - DNA, 22 bp, synthetic construct
  source 1..22: mol_type = other DNA
gctttacatcttccaggggtct

SEQ ID NO: 130 - DNA, 20 bp, synthetic construct
  source 1..20: mol_type = other DNA
gagcatgttcgcgttcccat

SEQ ID NO: 131 - DNA, 20 bp, synthetic construct
  source 1..20: mol_type = other DNA
cgcgcctcaatacggtagag

SEQ ID NO: 132 - DNA, 20 bp, synthetic construct
  source 1..20: mol_type = other DNA
ggggaactctctcgcaaggt

SEQ ID NO: 133 - DNA, 22 bp, synthetic construct
  source 1..22: mol_type = other DNA
cgacgggggctagaatcttaga

SEQ ID NO: 134 - DNA, 20 bp, synthetic construct
  source 1..20: mol_type = other DNA
gtcacagtcacagccacagc

SEQ ID NO: 135 - DNA, 19 bp, synthetic construct
  source 1..19: mol_type = other DNA
gctggatagcttgggagcg

SEQ ID NO: 136 - DNA, 22 bp, synthetic construct
  source 1..22: mol_type = other DNA
gccagctgctaagtccattgtc

SEQ ID NO: 137 - DNA, 21 bp, synthetic construct
  source 1..21: mol_type = other DNA
gtcggcactaggattgtctgc

SEQ ID NO: 138 - DNA, 21 bp, synthetic construct
  source 1..21: mol_type = other DNA
caaagttccactgggcaaacc

SEQ ID NO: 139 - DNA, 24 bp, synthetic construct
  source 1..24: mol_type = other DNA
tcccgttaataatgtccataggct

SEQ ID NO: 140 - DNA, 20 bp, synthetic construct
  source 1..20: mol_type = other DNA
atagggagcggaaggcacag

SEQ ID NO: 141 - DNA, 20 bp, synthetic construct
  source 1..20: mol_type = other DNA
agcagtacagacaggcagga

SEQ ID NO: 142 - DNA, 23 bp, synthetic construct
  source 1..23: mol_type = other DNA
agtctcatcaacatgctcttcct

SEQ ID NO: 143 - DNA, 18 bp, unidentified
  source 1..18: mol_type = other DNA
acccaggcagtggaggag

SEQ ID NO: 144 - DNA, 20 bp, synthetic construct
  source 1..20: mol_type = other DNA
cgctcgccatgagatatgct

SEQ ID NO: 145 - DNA, 21 bp, synthetic construct
  source 1..21: mol_type = other DNA
tacgcagtcaaacacaccagc

SEQ ID NO: 146 - DNA, 21 bp, synthetic construct
  source 1..21: mol_type = other DNA
ctgaaggtgtttgcatggtca

SEQ ID NO: 147 - DNA, 22 bp, synthetic construct
  source 1..22: mol_type = other DNA
ccatggattcaaagccgatacc

SEQ ID NO: 148 - DNA, 6546 bp, synthetic construct
  source 1..6546: mol_type = other DNA
gcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactatagaacgccacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggcccgcctctaccttgcagacccatataatataataactaaataagtaaataagacacacgcgagaacatatatacacaattacagtaacaataacaagaggacagatactaccaaaatgtgtggggaagcgggtaagctgccacagcaattaatgcacaacatttaacctacattcttccttatcggatcctcaaaacccttaaaaacatatgcctcaccctaacatattttccaattaaccctcaatatttctctgtcacccggcctctattttccattttcttctttacccgccacgcgtttttttctttcaaatttttttcttccttcttctttttcttccacgtcctcttgcataaataaataaaccgttttgaaaccaaactcgcctctctctctcctttttgaaatatttttgggtttgtttgatcctttccttcccaatctctcttgtttaatatatattcatttatatcacgctctctttttatcttcctttttttcctctctcttgtattcttccttcccctttctactcaaaccaagaagaaaaagaaaaggtcaatctttgttaaagaataggatcttctactacatcagcttttagatttttcacgcttactgcttttttcttcccaagatcgaaaatttactgaattaacagggccccccctcgaggtcgacggtatcgataagcttgaagcaagcctcctgaaagatgggtacccgcccatatgcttgccctgtcgagtcctgcgatcgccgcttttctcgccacgccaatcttacccgccatatccgcatccataccggtcagaagcccttccagtgtcgaatctgcatgcgtaacttcagtcgtaatgcgaaccttgtgcgccacatccgcacccacacaggatcccaaaagccgttccaatgtcggatctgtatgcggaactttagtcgaaaggccgacctgaggcgtcacattcgcacgcacaccggcgagaagccttttgcctgtgacatttgtgggaggaagtttgccaggaagggcgacctcaagaggcataccaaaatccatacaggtggcggaggcacacctgcagctgcgtcgactctagaggatccatctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcggagctccacttagacggcgaggacgtggcgatggcgcatgccgacgcgctagacgatttcgatctggacatgttgggggacggggattccccggggccgggatttaccccccacgactccgccccctacggcgctctggatatggccgacttcgagtttgagcagatgtttaccgatgcccttggaattgacgagtacggtgggtaggccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagg

SEQ ID NO: 149 - DNA, 10595 bp, synthetic construct
  source 1..10595: mol_type = other DNA
aaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttcccctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctactggcgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttcctttaccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcagtctactagtcagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaagatctgatatggatcgaattagatctcgccaccatggacaagaagtattctatcggactggacatcgggactaatagcgtcgggtgggccgtcatcactgacgagtacaaggtgccctctaagaagttcaaggtgctcgggaacaccgaccggcattccatcaagaaaaatctgatcggagctctcctctttgattcaggggagaccgctgaagcaacccgcctcaagcggactgctagacggcggtacaccaggaggaagaaccggatttgttaccttcaagagatattctccaacgaaatggcaaaggtcgacgacagcttcttccataggctggaagaatcattcctcgtggaagaggataagaagcatgaacggcatcccatcttcggtaatatcgtcgacgaggtggcctatcacgagaaatacccaaccatctaccatcttcgcaaaaagctggtggactcaaccgacaaggcagacctccggcttatctacctggccctggcccacatgattaagttcagaggccacttcctgatcgagggcgacctcaatcctgacaatagcgatgtggataaactgttcatccagctggtgcagacttacaaccagctctttgaagagaaccccatcaatgcaagcggagtcgatgccaaggccattctgtcagcccggctgtcaaagagccgcagacttgagaatcttatcgctcagctgccgggtgaaaagaaaaatggactgttcgggaacctgattgctctttcacttgggctgactcccaatttcaagtctaatttcgacctggcagaggatgccaagctgcaactgtccaaggacacctatgatgacgatctcgacaacctcctggcccagatcggtgaccaatacgccgaccttttccttgctgctaagaatctttctgacgccatcctgctgtctgacattctccgcgtgaacactgaaatcaccaaggcccctctttcagcttcaatgattaagcggtatgatgagcaccaccaggacctgaccctgcttaaggcactcgtccggcagcagcttccggagaagtacaaggaaatcttctttgaccagtcaaagaatggatacgccggctacatcgacggaggtgcctcccaagaggaattttataagtttatcaaacctatccttgagaagatggacggcaccgaagagctcctcgtgaaactgaatcgggaggatctgctgcggaagcagcgcactttcgacaatgggagcattccccaccagatccatcttggggagcttcacgccatccttcggcgccaagaggacttctacccctttcttaaggacaacagggagaagattgagaaaattctcactttccgcatcccctactacgtgggacccctcgccagaggaaatagccggtttgcttggatgaccagaaagtcagaagaaactatcactccctggaacttcgaagaggtggtggacaagggagccagcgctcagtcattcatcgaacggatgactaacttcgataagaacctccccaatgagaaggtcctgccgaaacattccctgctctacgagtactttaccgtgtacaacgagctgaccaaggtgaaatatgtcaccgaagggatgaggaagcccgcattcctgtcaggcgaacaaaagaaggcaattgtggaccttctgttcaagaccaatagaaaggtgaccgtgaagcagctgaaggaggactatttcaagaaaattgaatgcttcgactctgtggagattagcggggtcgaagatcggttcaacgcaagcctgggtacctaccatgatctgcttaagatcatcaaggacaaggattttctggacaatgaggagaaagaggacatccttgaggacattgtcctgactctcactctgttcgaggaccgggaaatgatcgaggagaggcttaagacctacgcccatctgttcgacgataaagtgatgaagcaacttaaacggagaagatataccggatggggacgccttagccgcaaactcatcaacggaatccgggacaaacagagcggaaagaccattcttgatttccttaagagcgacggattcgctaatcgcaacttcatgcaacttatccatgatgattccctgacctttaaggaggacatccagaaggcccaagtgtctggacaaggtgactcactgcacgagcatatcgcaaatctggctggttcacccgctattaagaagggtattctccagaccgtgaaagtcgtggacgagctggtcaaggtgatgggtcgccataaaccagagaacattgtcatcgagatggccagggaaaaccagactacccagaagggacagaagaacagcagggagcggatgaaaagaattgaggaagggattaaggagctcgggtcacagatccttaaagagcacccggtggaaaacacccagcttcagaatgagaagctctatctgtactaccttcaaaatggacgcgatatgtatgtggaccaagagcttgatatcaacaggctctcagactacgacgtggaccacatcgtccctcagagcttcctcaaagacgactcaattgacaataaggtgctgactcgctcagacaagaaccggggaaagtcagataacgtgccctcagaggaagtcgtgaaaaagatgaagaactattggcgccagcttctgaacgcaaagctaatcactcagcggaagttcgacaatctcactaaggctgagaggggcggactgagcgaactggacaaagcaggattcattaaacggcaacttgtggagactcggcagattactaaacatgtagcccaaatccttgactcacgcatgaataccaagtacgacgaaaacgacaaacttatccgcgaggtgaaggtgattaccctgaagtccaagctggtcagcgatttcagaaaggactttcaattctacaaagtgcgggagatcaataactatcatcatgctcatgacgcatatctgaatgccgtggtgggaaccgccctaatcaagaagtacccaaagctggaaagcgagttcgtgtacggagactacaaggtctacgacgtgcgcaagatgattgccaaatctgagcaggagatcggaaaggccaccgcaaagtacttcttctacagcaacatcatgaatttcttcaagaccgaaatcacccttgcaaacggtgagatccggaagaggccgctcatcgagactaatggggagactggcgaaatcgtgtgggacaagggcagagatttcgctaccgtgcgcaaagtgctttctatgcctcaagtgaacatcgtgaagaaaaccgaggtgcaaaccggaggcttttctaaggaatcaatcctccccaagcgcaactccgacaagctcattgcaaggaagaaggattgggaccctaagaagtacggcggattcgattcaccaactgtggcttattctgtcctggtcgtggctaaggtggaaaaaggaaagtctaagaagctcaagagcgtgaaggaactgctgggtatcaccattatggagcgcagctccttcgagaagaacccaattgactttctcgaagccaaaggttacaaggaagtcaagaaggaccttatcatcaagctcccaaagtatagcctgttcgaactggagaatgggcggaagcggatgctcgcctccgctggcgaacttcagaagggtaatgagctggctctcccctccaagtacgtgaatttcctctaccttgcaagccattacgagaagctgaaggggagccccgaggacaacgagcaaaagcaactgtttgtggagcagcataagcattatctggacgagatcattgagcagatttccgagttttctaaacgcgtcattctcgctgatgccaacctcgataaagtccttagcgcatacaataagcacagagacaaaccaattcgggagcaggctgagaatatcatccacctgttcaccctcaccaatcttggtgcccctgccgcattcaagtacttcgacaccaccatcgaccggaaacgctatacctccaccaaagaagtgctggacgccaccctcatccaccagagcatcaccggactttacgaaactcggattgacctctcacagctcggaggggatgagggagctcccaagaaaaagcgcaaggtaggtagttccggatcctagactctcgaggcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttctctagagcggccgtggtatcgtccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccg

SEQ ID NO: 150 - DNA, 4887 bp, synthetic construct
  source 1..4887: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccacgcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaattgtaatacgactcactatagggcgaattggagctccaccgcggtggcggccgctctagaactagtggatcccccgggctgcaggaattcgatatcaagcttatcgataccgtcgacctcgagggggggcccggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 151 - DNA, 5458 bp, synthetic construct
  source 1..5458: mol_type = other DNA
ctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtaagatctgtttagcttgccttgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctcgaattcatcgatgtcagttattaccctgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaattgtaatacgactcactatagggcgaattggagctccaccgcggtggcggccgctctagaactagtggatcccccgggctgcaggaattcgatatcaagcttatcgataccgtcgacctcgagggggggcccggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttt

SEQ ID NO: 152 - DNA, 20 bp, synthetic construct
  source 1..20: mol_type = other DNA
ggacaaaattcctgatacgg

SEQ ID NO: 153 - DNA, 20 bp, synthetic construct
  source 1..20: mol_type = other DNA
ggatgacgatgaacaagacg

SEQ ID NO: 154 - DNA, 20 bp, synthetic construct
  source 1..20: mol_type = other DNA
gcgtagggaacgtcgcaaag

SEQ ID NO: 155 - DNA, 20 bp, synthetic construct
  source 1..20: mol_type = other DNA
ccgcacagatgcgtaaggta

SEQ ID NO: 156 - DNA, 20 bp, synthetic construct
  source 1..20: mol_type = other DNA
ggtgtggaaaagctccctaa

SEQ ID NO: 157 - DNA, 20 bp, synthetic construct
  source 1..20: mol_type = other DNA
tatccctactcactcaaagg

SEQ ID NO: 158 - DNA, 57 bp, synthetic construct
  source 1..57: mol_type = other DNA
tgtgtggtgtgtggtgtgtgtgggtgtgtggtgtgtgggtctgtaagcggatgccgg

SEQ ID NO: 159 - DNA, 79 bp, synthetic construct
  source 1..79: mol_type = other DNA
ctccttacgcatctgtgcgtaccctttaaaatctcattggctcgtgattaatttgttctgtgctgctgaatattcatgc

SEQ ID NO: 160 - DNA, 60 bp, synthetic construct
  source 1..60: mol_type = other DNA
ttacatatcctctacaccgagcgcgtcgacccgtcgaatggtttagcttgccttgtcccc

SEQ ID NO: 161 - DNA, 60 bp, synthetic construct
  source 1..60: mol_type = other DNA
ggcggcgttagtatcgaatccacccaccacacacacccacacacaccacacacccaccca

SEQ ID NO: 162 - DNA, 60 bp, synthetic construct
  source 1..60: mol_type = other DNA
gggctataaagtatatatagatacaaatatatgatgaatcgtttagcttgccttgtcccc

SEQ ID NO: 163 - DNA, 60 bp, synthetic construct
  source 1..60: mol_type = other DNA
gaataaaatttcccagcacgactttgatcacccgaaagggggcggcgttagtatcgaatc

SEQ ID NO: 164 - DNA, 120 bp, synthetic construct
  source 1..120: mol_type = other DNA
taatacgactcactatagnnnnnnnnnnnnnnnnnnngttttagagctagaaatagcaagttaaaataaggctagtccgttatcaacttgaaaaagtggcaccgagtcggtgcttttttt

SEQ ID NO: 165 - RNA, 103 nt, synthetic construct
  source 1..103: mol_type = other RNA
  modified_base 22..25: mod_base = OTHER; note = Uracil
  modified_base 31: mod_base = OTHER; note = Uracil
  modified_base 37: mod_base = OTHER; note = Uracil
  modified_base 44..45: mod_base = OTHER; note = Uracil
  modified_base 50: mod_base = OTHER; note = Uracil
  modified_base 56: mod_base = OTHER; note = Uracil
  modified_base 59: mod_base = OTHER; note = Uracil
  modified_base 63..64: mod_base = OTHER; note = Uracil
  modified_base 66: mod_base = OTHER; note = Uracil
  modified_base 71..72: mod_base = OTHER; note = Uracil
  modified_base 80: mod_base = OTHER; note = Uracil
  modified_base 90: mod_base = OTHER; note = Uracil
  modified_base 94: mod_base = OTHER; note = Uracil
  modified_base 97..103: mod_base = OTHER; note = Uracil
nnnnnnnnnnnnnnnnnnnngttttagagctagaaatagcaagttaaaataaggctagtccgttatcaacttgaaaaagtggcaccgagtcggtgcttttttt

SEQ ID NO: 166 - DNA, 455 bp, synthetic construct
  source 1..455: mol_type = other DNA
gggcgaattcgtattttacgcgcaagccggacccgcgcttgcacggctgaggtgcgtagggaacgtcgcaaagaggcatgaatattcagcagcacagaacaaattaatcacttaattaatagaaacggctgacaaccattttgttagtcctcggaaaatctccgaacaaatgagaaagcaaaatataataacacagagtttggcgagaagaagatatatctcgagagtcctaggaagatgaagatgtgtctggctttagtaatcattatattgcttgttgtaatcatcgtcgccattgctgttcactttagtcgatagaaattcactcggaataggatcccttacatatcctctacaccgagcgcgtcgacccgtcgaatgaatgtgagttgcgtgttgttccttcggaaatgttgctaaaaaacagagccattccttttgcaggcatgcaatta

(19) United States
(12) Patent Application Publication
(10) Pub. No.: US 2026/0152752 A1
(43) Pub. Date: Jun. 4, 2026

(54) ASSEMBLING SYNTHETIC DNA CONSTRUCTS FROM NATURAL DNA

(71) Applicant: UNIVERSITY OF SOUTHERN CALIFORNIA, Los Angeles, CA (US)

(72) Inventors: Alessandro Venega CORADINI, Los Angeles, CA (US); Ian EHRENREICH, Los Angeles, CA (US)

(73) Assignee: UNIVERSITY OF SOUTHERN CALIFORNIA, Los Angeles, CA (US)

(21) Appl. No.: 18/861,973

(22) PCT Filed: May 3, 2023
(86) PCT No.: PCT/US2023/020865

§ 371 Date: Oct. 31, 2024

Related U.S. Application Data
(60) Provisional application No. 63/337,891, filed on May 3, 2022.

(51) Int. Cl. C12N 15/66; C12N 1/08; C12N 1/14; C12N 1/20; C12N 5/071; C12N 5/10; C12N 9/22; C12N 15/10; C12N 15/113; C12N 15/80

(52) U.S. Cl. CPC C12N 15/66; C12N 1/08; C12N 1/14; C12N 1/20; C12N 5/0602; C12N 5/10; C12N 9/222; C12N 15/10; C12N 15/113; C12N 15/80; C12N 2310/20

(57) ABSTRACT

The disclosure provides for methods of constructing synthetic chromosomes including the steps of providing host cells with an endogenous chromosome, transforming a cloning vector and a cloning cassette into the host cells, excising target genomic nucleic acids from the endogenous chromosome, recombining the excised target genomic nucleic acids with the cloning cassette via homologous recombination to form heterologous vectors comprising cloned sequences, extracting the heterologous vectors containing the cloned sequences from the host cells, digesting the heterologous vectors with a restriction endonuclease to release the cloned sequences from the heterologous vectors to provide released cloned sequences, and introducing the released cloned sequences, a centromere cassette, and a yeast artificial chromosomes or bacterial artificial chromosomes into a second host cell, such that the released cloned sequences, the centromere cassette, and the YAC or BAC recombine with one another via homologous recombination to produce the synthetic chromosome.

### RELATED APPLICATIONS

This application claims priority under 35 U.S.C. § 119(e) to U.S. Provisional Patent Application No. 63/337,891, filed May 3, 2022, which is incorporated herein by reference.

### GOVERNMENT SUPPORT

This invention was made with government support under Grant Number R35GM130381, awarded by the National Institutes of Health, and Grant Number 2124400, awarded by the National Science Foundation. The government has certain rights in the invention.

### BACKGROUND OF THE INVENTION

It is now possible to answer fundamental questions in biology by synthesizing chromosomes. For example, a longstanding question has been what is the minimal set of genes required to produce a living cell? To answer this question, researchers used design-build-test cycles to synthesize a Mycoplasma mycoides chromosome that contains only 473 genes and is still capable of producing a free-living bacterium that replicates on lab timescales. Among the genes in this minimal set, 83% functioned in the expression and preservation of genetic information, the cell membrane, or cytosolic metabolism, while 17% had unknown functions. Identification of this minimal gene set demonstrates the potential for using chromosome synthesis to understand mechanisms underlying cellular life and its diversity.

To date, synthetic chromosomes have exclusively been generated de novo, through the progressive assembly of small synthetic fragments of DNA into larger molecules by a combination of in vitro and in vivo techniques. De novo synthesis is powerful because it allows the complete reprogramming of a chromosome's sequence and structure. For example, de novo chromosome synthesis was used to generate an Escherichia coli strain in which all 18,214 instances of three codons were synonymously reprogrammed, resulting in a strain that utilizes only 61 codons. In another example, the Sc2.0 community is using de novo chromosome synthesis to generate a strain of the model budding yeast Saccharomyces cerevisiae in which all transposable elements have been eliminated and LoxP sites have been incorporated between genes, enabling the generation of random chromosome rearrangements by Cre recombinase.

The substantial amount of DNA fragment synthesis and assembly involved in de novo chromosome synthesis limits its use in biological research. Reductions in labor and reagent costs are needed to enable biologists to employ chromosome synthesis more widely. As such, there is a need for new methods of building synthetic chromosomes from cloned segments of natural DNA could be a relatively cheap and fast alternative to de novo chromosome synthesis. Such a method would enable the use of chromosome synthesis in research that does not require complete chromosome reprogramming. For example, projects that could be enabled include mapping the genetic basis of trait differences between individuals and species, probing the structural requirements of chromosomes, and streamlining chromosomes through the multiplex deletion of non-essential regions. The present disclosure satisfies these needs.

### SUMMARY OF THE INVENTION

This disclosure introduces the CReATiNG (Cloning, Reprogramming, and Assembling Tiled Natural Genomic DNA) method for building synthetic chromosomes from natural components in S. cerevisiae. The first step of CReATiNG is the cloning of natural chromosome segments such that unique adapter sequences are appended to their termini, specifying how these molecules will recombine with each other later when they are assembled. The second step of CReATiNG is co-transforming cloned segments into cells and assembling them by homologous recombination in vivo. Synthetic chromosomes generated with CReATiNG can replace the native chromosomes in cells, making it possible to directly test their phenotypic effects. Here, we describe CReATiNG and demonstrate several of its use cases.

Accordingly, the disclosure provides methods for constructing one or more synthetic chromosomes as described herein. In one aspect, a method for constructing a synthetic chromosome comprises the steps of: providing one or more host cells comprising one or more endogenous chromosomes; transforming one or more cloning vectors and one or more cloning cassettes into the one or more host cells; excising one or more target genomic nucleic acids from the one or more endogenous chromosomes; recombining the excised target genomic nucleic acids with the one or more cloning cassettes via homologous recombination to form one or more heterologous vectors comprising one or more cloned sequences; extracting the one or more heterologous vectors containing the one or more cloned sequences from the host cells; digesting the one or more heterologous vectors with a restriction endonuclease to release the one or more cloned sequences from the heterologous vectors to provide one or more released cloned sequences; and introducing the one or more released cloned sequences, one or more centromere cassettes, and one or more yeast artificial chromosomes or bacterial artificial chromosomes into a second host cell, wherein the one or more released cloned sequences, the one or more centromere cassettes, and the one or more yeast artificial chromosomes or bacterial artificial chromosomes recombine with one another via homologous recombination to produce the synthetic chromosome.

In one aspect, the host cells express a Cas9 endonuclease protein; and wherein the transformation step further comprises transforming into the one or more host cells one or more guide ribonucleic acids (gRNAs) that are complementary to regions flanking the one or more target genomic nucleic acid sequences of the one or more endogenous chromosome, wherein the Cas9 endonuclease protein and the gRNAs excise the one or more target genomic nucleic acid sequences from the one or more endogenous chromosomes. While embodiments associated with this disclosure exemplify the use of Cas9, other DNA-targeting Cas enzymes, such as Cas12a/Cpf1, are also compatible with CReATiNG.

In one aspect, each of the one or more cloning cassettes comprise two separate linear segments of nucleic acid, wherein a first segment of nucleic acid comprises a first hook region and a second segment of nucleic acid comprises a second hook region, wherein the first hook region comprises formula A-B-C, wherein: A comprises a unique restriction endonuclease site and first homology region comprising a nucleic acid sequence complementary to first region of the cloning vector; B comprises a first adapter region comprising a nucleic acid sequence complementary to another adapter region; and C comprises a first complementary region comprising a nucleic acid sequence complementary to a region flanking the excised target genomic nucleic acids; the second hook region comprises formula D-E-F, wherein: D comprises a second complementary region comprising a nucleic acid sequence complementary to a region flanking the excised target genomic nucleic acids; E comprises a second adapter region comprising a nucleic acid sequence complementary to another adapter region; and F comprises a unique restriction endonuclease site and a second homology region comprising a nucleic acid sequence complementary to second region of the cloning vector; and wherein the two separate pieces of nucleic acid are co-transformed into the host cells.

In another aspect, wherein the one or more cloning vectors comprise the cloning cassette, the cloning cassette comprises a first hook region, a second hook region, and a multiple cloning site (MCS) disposed there between, wherein the first hook region comprises formula A-B-C, wherein: A comprises a unique restriction endonuclease site; B comprises a first adapter region comprising a nucleic acid sequence complementary to another adapter region; and C comprises a first complementary region comprising a nucleic acid sequence complementary to a region flanking the excised target genomic nucleic acids; the second hook region comprises formula D-E-F, wherein: D comprises a second complementary region comprising a nucleic acid sequence complementary to a region flanking the excised target genomic nucleic acids; E comprises a second adapter region comprising a nucleic acid sequence complementary to another adapter region; and F comprises a unique restriction endonuclease site; and wherein prior to the transformation step, the cloning cassette is linearized by restriction endonuclease digestion using a restriction endonuclease that recognizes and cuts a nucleic acid sequence within the MCS.

In another aspect, the recombining step further comprises the first complementary region and the second complementary region separately contacting and binding to complementary nucleic acid sequences flanking the one or more excised target genomic nucleic acids; and the first homology region and the second homology region separately contacting and binding to the first region and second region of the one or more cloning vectors, respectively.

In another aspect, the transforming step further comprises introducing one or more repair templates into the host cells, wherein the one or more repair templates comprise: a selectable marker; a first homology arm; and a second homology arm; wherein the first homology arm and the second homology are complementary to nucleic acid sequences of the one or more endogenous chromosome that flank the one or more target genomic nucleic acids to permit homologous recombination between the one or more repair templates and the one or more endogenous chromosomes, thereby repairing the endogenous chromosome.

In another aspect, during the digesting step, a restriction endonuclease recognizes and cuts unique restriction endonuclease sites A and F flanking the one or more cloning cassettes, thereby releasing the one or more cloned sequences from the one or more heterologous vectors to provide one or more released cloned sequences. In another aspect, the one or more released cloned sequences comprise structure B-CS-E, wherein CS comprises the one or more cloned sequences.

In another aspect, during the introducing step, the first adapter region and the second adapter region of a first released clone sequence are complementary to and binds to one of the first adapter sequence and the second adapter sequence of a second released cloned sequence to permit homologous recombination between the first released clone sequence and the second released clone sequence to produce the synthetic chromosome.

In another aspect, some embodiments of methods of making synthetic chromosomes also may comprise the use of centromere cassettes to form the synthetic chromosome. The centromere cassette comprises an origin of replication, a centromere sequence, and an adapter arm flanking both the origin of replication and the centromere sequence, and optionally, a selectable marker; wherein the adapter arm is complementary to another adapter region of the cloning cassette.

In another aspect, the synthetic chromosome described herein may comprise deletion of segments of nucleic acid as compared to the endogenous chromosome; or wherein the synthetic chromosome comprises a rearrangement of nucleic acid segments as compared to an arrangement of corresponding nucleic acid segments of the endogenous chromosome.

In another aspect, the one or more host cells are cells of at least two different genera or species, wherein the synthetic chromosome is a chimeric chromosome comprising nucleic acid sequences from the at least two different genera or species. In another aspect, the one or more host cells are one or more of a human cell, a fungal cell, or a bacterial cell.

These and other features and advantages of this invention will be more fully understood from the following detailed description of the invention taken together with the accompanying claims. It is noted that the scope of the claims is defined by the recitations therein and not by the specific discussion of features and advantages set forth in the present description.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1A-D. Synthesizing chromosomes from natural components in yeast using CReATiNG. a. The Bacterial Artificial Chromosome/Yeast Artificial Chromosome (BAC/YAC) vector (pASC1) used for cloning natural chromosome segments in vivo. Homology may be flanked by sequence adapters that program how a segment will assemble with others in later steps. b. A segment is cloned by co-transforming a linearized cloning vector, gRNAs targeting both sides of the segment, and a selectable repair template into a donor cell constitutively expressing Cas9. Cloned segments are then extracted from yeast and transferred to the E. coli. c. Cloned segments are excised from the vector through restriction digestion with I-SceI. These molecules are then purified and co-transformed into a recipient yeast cell with a centromere cassette and a centromere-free version of the BAC/YAC cloning vector (pASC2). These molecules are assembled into a synthetic chromosome by homologous recombination in vivo, while the native chromosome is eliminated by centromere destabilization and counterselection. d. Oxford Nanopore Technologies sequencing confirmed the correct assembly of Saccharomyces paradoxus ChrI (white) and replacement of the native S. cerevisiae ChrI (black) in BY. The plot shows reads mapped to each chromosome using a reference genome including both BY and S. paradoxus ChrI.

FIG. 2A-F. Recombining chromosomes between strains and species using CReATiNG. a. Segments 1 through 3 were cloned from two additional strains, the BY (dark gray) and RM (light gray) strains of S. cerevisiae. The 27 possible syntenic combinations of the S. paradoxus (white), BY, and RM segments were then assembled in BY recipient cells and the native ChrI was eliminated. b. Representative validations of assembled chromosomes by Oxford Nanopore Technologies sequencing. In silico designs of each chromosome are shown with a subset of mapped reads plotted below them. c and d. Euploid strains carrying the synthetic chromosomes were phenotyped for doubling time in rich medium containing glucose at 30° C. and 35° C., respectively. Means are shown for each strain, with error bars representing one standard deviation around the mean. e. The mean effect (horizontal line) of each segment across all genotypes at 30° C. is shown, with dots representing the mean of a genotype across 12 replicates. f. The mean effect (horizontal line) of each segment across all genotypes at 35° C. is shown, with dots representing the mean of a genotype across 12 replicates.

FIG. 3A-B. Restructuring ChrI with CReATiNG. a. Five possible restructured versions of BY ChrI were created by altering the adapters appended to each segment. Oxford Nanopore sequencing was used to confirm that the chromosome assemblies had the correct structure. In silico designs of each chromosome are shown. b. Growth analysis of the natural and five non-natural chromosome structures in rich medium containing glucose at 30° C. Means are shown for each strain, with error bars representing one standard deviation around the mean.

FIG. 4A-D. Multiplex gene deletion with CReATiNG. a. We attempted to delete 10 non-adjacent regions of the chromosome core and both subtelomeres from BY ChrI, totaling 39.9% of the chromosome. To do this, we cloned 11 segments of the ChrI, assembled them, and performed native chromosome elimination. b. Oxford Nanopore Technologies sequencing of a colony confirmed results from PCR checks. The colony with the most deletions (nine regions) had the correct structure, but had retained the region containing SYN8. An in silico design of the chromosome is shown with a subset of mapped reads plotted below it. c. Growth rate analysis of different BY strains, including the unaltered reference strain (BY), a strain carrying a synthetic circular ChrI lacking subtelomeres (BY synthetic ChrI), a synthetic circular ChrI lacking nine core regions and both subtelomeres (multiple deletion ChrI), a synthetic circular ChrI lacking nine core regions, both subtelomeres, and SYN8 (multiple deletion ChrI syn8Δ), and the reference strain with SYN8 deleted (BY syn8Δ). Means are shown for each strain, with error bars representing one standard deviation around the mean. d. Recombination between the synthetic ChrI and the native ChrI resulted in the synthetic chromosome containing SYN8.

FIG. 5A-D. Reagents used for cloning in CReATiNG. a. DNA templates for producing gRNAs by in vitro transcription are generated by PCR. An oligonucleotide containing the tracrRNA is amplified using a tailed forward primer containing a 20 nt target sequence and a T7 promoter (SEQ ID NO: 164)(top sequence). The PCR reaction generates a dsDNA template that is transcribed in vitro into gRNAs (SEQ ID NO: 165) (bottom sequence) by T7 RNA polymerase. T7 promoter sequence (underlined); target sequence (bold); scaffold oligo and tracrRNA (italics) b. Map of the BAC/YAC vector pASC1. The vector contains a cloning site flanked by I-SceI where a cloning cassette is inserted using restriction digestion and ligation. c. Example of a cloning cassette design for capture of S. paradoxus segment 1. The cassette (SEQ ID NO: 166) contains segment-specific homology arms (underlined sequences) that are flanked by adapters (bold sequences) that program how cloned segments will recombine during chromosome assembly. Once the cloning cassette is added to pASC1, the vector is linearized by restriction digestion, exposing the homology arms. d. Map of the modified pRS316 vector (pMM_KanMX) used as a template for amplification of the KanMX repair template. Primers bind to the sites indicated as repair template priming sites.

FIG. 6A-D. The cloning step of CReATiNG has high efficiency. a. Map of the strategy for cloning the core of Saccharomyces paradoxus ChrI as three segments. b. Transformed cells are selected in SC plates lacking uracil and containing G418. c. Map of the pASC1 cloning vector containing segment 1 from Saccharomyces paradoxus ChrI. The black arrows indicate primer sites for PCR junction checks. d. Cloning efficiency was accessed using junction PCRs. Electrophoresis in a 1% agarose gel shows the expected DNA bands for both junctions in 100% (5 of 5) checked colonies.

FIG. 7A-C. Assembly of a synthetic S. paradoxus ChrI in BY. a. The three segments of S. paradoxus ChrI were liberated from the cloning vector by I-SceI digestion and separated from the vector in an 0.5% agarose gel. b. The three segments, the assembly vector (pASC2), and a centromere cassette were co-transformed into and assembled in BY (white). The native BY ChrI (black) was marked for elimination prior to assembly. c. Correct assembly of S. paradoxus ChrI and elimination of the native ChrI were initially confirmed by diagnostic PCRs targeting both chromosomes. The gel pictures on the left show junction PCR results for native BY ChrI before (upper) and after (bottom) native ChrI elimination. The gel pictures on the right show junction PCR results for the S. paradoxus ChrI before (upper) and after (bottom) native ChrI elimination.

FIG. 8A-B. S. paradoxus ChrI linearization using CRISPR Cas9. a. We assembled chromosomes as circular molecules. To linearize the synthetic S. paradoxus ChrI, we targeted the junctions between the chromosome and the vector with CRISPR/Cas9. We also provided repair templates containing synthetic telomere seed sequences (TSS) and selectable markers, URA3 and (NatMX) for the left and right arms, respectively. b. Plate images showing cells in which the circular S. paradoxus ChrI was converted to its linear form. Cells with the linear ChrI become histidine auxotrophs but are uracil prototrophs with resistance to nourseothricin.

### DETAILED DESCRIPTION OF THE INVENTION

De novo chromosome synthesis is costly and time-consuming, limiting its use in research and biotechnology. Building synthetic chromosomes from natural components is an unexplored alternative with many potential applications. Here, we report CReATiNG (Cloning, Reprogramming, and Assembling Tiled Natural Genomic DNA), a method for constructing synthetic chromosomes from natural components in yeast. CReATiNG entails cloning segments of natural chromosomes and then programmably assembling them into synthetic chromosomes that can replace the native chromosomes in cells. We used CReATiNG to synthetically recombine chromosomes between strains and species, to modify chromosome structure, and to delete many linked, non-adjacent regions totaling 39% of a chromosome. The multiplex deletion experiment revealed that CReATiNG also enables recovery from flaws in synthetic chromosome design via recombination between a synthetic chromosome and its native counterpart. CReATiNG facilitates the application of chromosome synthesis to diverse biological problems.

### Definitions

The following definitions are included to provide a clear and consistent understanding of the specification and claims. Unless defined otherwise, technical and scientific terms used herein have the same meaning as commonly understood by one of ordinary skill in the art to which the present disclosure belongs. See, e.g., Singleton et al., Dictionary of Microbiology and Molecular Biology 2nd ed., J. Wiley & Sons (New York, N.Y. 1994); Sambrook et al., Molecular Cloning, A Laboratory Manual, Cold Springs Harbor Press (Cold Springs Harbor, N.Y. 1989).

The methods described herein may employ, unless otherwise indicated, conventional techniques and descriptions of molecular biology (including recombinant techniques), cell biology, biochemistry, and cellular engineering technology, all of which are within the skill ofthose who practice in the art. Such conventional techniques include oligonucleotide synthesis, hybridization and ligation of oligonucleotides, transformation and transduction of cells, engineering of recombination systems, creation of transgenic animals and plants, and human gene therapy. Specific illustrations of suitable techniques can be had by reference to the examples herein. However, equivalent conventional procedures can, of course, also be used. Such conventional techniques and descriptions can be found in standard laboratory manuals such as Genome Analysis: A Laboratory Manual Series (Vols. I-IV) (Green, et al., eds., 1999); Genetic Variation: A Laboratory Manual (Weiner, et al., eds., 2007); Sambrook and Russell, Condensed Protocols from Molecular Cloning: A Laboratory Manual (2006); and Sambrook and Russell, Molecular Cloning: A Laboratory Manual (2002) (all from Cold Spring Harbor Laboratory Press); Protein Methods (Bollag et al., John Wiley & Sons 1996); Nonviral Vectors for Gene Therapy (Wagner et al. eds., Academic Press 1999); Viral Vectors (Kaplift & Loewy, eds., Academic Press 1995); Immunology Methods Manual (Lefkovits ed., Academic Press 1997); Gene Therapy Techniques, Applications and Regulations From Laboratory to Clinic (Meager, ed., John Wiley & Sons 1999); M. Giacca, Gene Therapy (Springer 2010); Gene Therapy Protocols (LeDoux, ed., Springer 2008); Cell and Tissue Culture: Laboratory Procedures in Biotechnology (Doyle & Griffiths, eds., John Wiley & Sons 1998); Mammalian Chromosome Engineering—Methods and Protocols (G. Hadlaczky, ed., Humana Press 2011); Essential Stem Cell Methods, (Lanza and Klimanskaya, eds., Academic Press 2011); Stem Cell Therapies: Opportunities for Ensuring the Quality and Safety of Clinical Offerings: Summary of a Joint Workshop (Board on Health Sciences Policy, National Academies Press 2014); Essentials of Stem Cell Biology, Third Ed., (Lanza and Atala, eds., Academic Press 2013); and Handbook of Stem Cells, (Atala and Lanza, eds., Academic Press 2012), all of which are herein incorporated by reference in their entirety for all purposes. Before the present compositions, research tools and methods are described, it is to be understood that this invention is not limited to the specific methods, compositions, targets and uses described, as such may, of course, vary. It is also to be understood that the terminology used herein is for the purpose of describing particular aspects only and is not intended to limit the scope of the present invention, which will be limited only by the appended claims.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five substituents on the ring.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

This disclosure provides ranges, limits, and deviations to variables such as volume, mass, percentages, ratios, etc. It is understood by an ordinary person skilled in the art that a range, such as “number 1” to “number 2”, implies a continuous range of numbers that includes the whole numbers and fractional numbers. For example, 1 to 10 means 1, 2, 3, 4, 5, . . . 9, 10. It also means 1.0, 1.1, 1.2. 1.3, . . . , 9.8, 9.9, 10.0, and also means 1.01, 1.02, 1.03, and so on. If the variable disclosed is a number less than “number 10”, it implies a continuous range that includes whole numbers and fractional numbers less than number 10, as discussed above. Similarly, if the variable disclosed is a number greater than “number 10”, it implies a continuous range that includes whole numbers and fractional numbers greater than number 10. These ranges can be modified by the term “about”, whose meaning has been described above.

The recitation of a), b), c), . . . or i), ii), iii), or the like in a list of components or steps do not confer any particular order unless explicitly stated. One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms. The disclosure illustratively described herein may be suitably practiced in the absence of any element or elements, limitation, or limitations not specifically disclosed herein.

The terms “polynucleotide” and “nucleic acid” are used interchangeably and mean at least two or more ribo- or deoxy-ribo nucleic acid base pairs (nucleotide) linked which are through a phosphoester bond or equivalent. The nucleic acid includes polynucleotide and polynucleoside. The nucleic acid includes a single molecule, a double molecule, a triple molecule, a circular molecule, or a linear molecule. Examples of the nucleic acid include RNA, DNA, cDNA, a genomic nucleic acid, a naturally existing nucleic acid, and a non-natural nucleic acid such as a synthetic nucleic acid but are not limited. Short nucleic acids and polynucleotides (e.g., 10 to 20, 20 to 30, 30 to 50, 50 to 100 nucleotides) are commonly called “oligonucleotides” or “probes” of single-stranded or double-stranded DNA.

As used herein, “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The term “substantial identity” in the context of a peptide indicates that a peptide comprises a sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, or 94%, or even 95%, 96%, 97%, 98% or 99%, sequence identity to the reference sequence over a specified comparison window. In certain embodiments, optimal alignment is conducted using the homology alignment algorithm of Needleman and Wunsch (Needleman and Wunsch, JMB, 48, 443 (1970)). An indication that two peptide sequences are substantially identical is that one peptide is immunologically reactive with antibodies raised against the second peptide. Thus, a peptide is substantially identical to a second peptide, for example, where the two peptides differ only by a conservative substitution. Thus, embodiment of the invention also provides nucleic acid molecules and peptides that are substantially identical to the nucleic acid molecules and peptides presented herein.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

A “chromosome” is a nucleic acid molecule—and associated proteins—that is capable of replication and segregation in a cell upon division of the cell. Typically, a chromosome contains a centromeric region, replication origins, telomeric regions and a region of nucleic acid between the centromeric and telomeric regions. An “acrocentric chromosome” refers to a chromosome with arms of unequal length.

“Synthetic chromosomes” (also referred to as “artificial chromosomes”) are nucleic acid molecules, typically DNA, that stably replicate and segregate alongside endogenous chromosomes in cells that have the capacity to accommodate and express heterologous genes. A “mammalian synthetic chromosome” refers to chromosomes that have an active mammalian centromere(s). A “human synthetic chromosome” refers to a chromosome that includes a centromere that functions in human cells and that preferably is produced in human cells. Some exemplary artificial chromosomes are described in, for example, U.S. Pat. Nos. 8,389,802; 7,521,240; 6,025,155; 6,077,697; and U.S. Pat. No. 5,891,691.

“Endogenous chromosomes” refers to chromosomes found in a cell prior to generation or introduction of a synthetic chromosome.

“Site-specific recombination” refers to site-specific recombination that is affected between two specific sites on a single nucleic acid molecule or between two different molecules that requires the presence of an exogenous protein, such as an integrase or recombinase. Certain site-specific recombination systems can be used to specifically delete, invert, or insert DNA, with the precise event controlled by the orientation of the specific sites, the specific system and the presence of accessory proteins or factors.

The term “genetically engineered” may refer to any manipulation of a host cell's genome (e.g., by insertion or deletion of nucleic acids). The term “genetically edited” refers to a host cell whose genome has been edited by a CRISPR complex.

As used herein, the term “gene” refers to any segment of DNA associated with a biological function. Thus, genes include, but are not limited to, promoter sequences, terminator sequences, splice sites, polyubiquitination sites, intron sequences, coding sequences and/or the regulatory sequences required for their expression. Genes can also include non-expressed DNA segments that, for example, form recognition sequences for other proteins, such as a guide RNA. Genes can be obtained from a variety of sources, including cloning from a source of interest or synthesizing from known or predicted sequence information, and may include sequences designed to have desired parameters.

As used herein, the term “homologous” or “homologue” or “ortholog” is known in the art and refers to related sequences that share a common ancestor or family member and are determined based on the degree of sequence identity. The terms “homology,” “homologous,” “substantially similar” and “corresponding substantially” are used interchangeably herein. They refer to nucleic acid fragments wherein changes in one or more nucleotide bases do not affect the ability of the nucleic acid fragment to mediate gene expression or produce a certain phenotype. These terms also refer to modifications of the nucleic acid fragments of the instant disclosure such as deletion or insertion of one or more nucleotides that do not substantially alter the functional properties of the resulting nucleic acid fragment relative to the initial, unmodified fragment. It is therefore understood, as those skilled in the art will appreciate, that the disclosure encompasses more than the specific exemplary sequences. These terms describe the relationship between a gene found in one species, subspecies, variety, cultivar or strain and the corresponding or equivalent gene in another species, subspecies, variety, cultivar or strain. For purposes of this disclosure, homologous sequences are compared. “Homologous sequences”, “homologues”, or “orthologs” are thought, believed, or known to be functionally related. A functional relationship may be indicated in any one of a number of ways, including, but not limited to: (a) degree of sequence identity and/or (b) the same or similar biological function. In some embodiments, both (a) and (b) are indicated. Homology can be determined using software programs readily available in the art, such as those discussed in Current Protocols in Molecular Biology (F. M. Ausubel et al., eds., 1987) Supplement 30, section 7.718, Table 7.71. Some alignment programs are MacVector (Oxford Molecular Ltd, Oxford, U.K.), ALIGN Plus (Scientific and Educational Software, Pennsylvania) and AlignX (Vector NTI, Invitrogen, Carlsbad, Calif). Another alignment program is Sequencher (Gene Codes, Ann Arbor, Mich.), using default parameters.

The term “complement” or “complementary” as used herein means the complementary sequence to a nucleic acid according to standard Watson/Crick base pairing rules. A complement sequence can also be a sequence of RNA complementary to the DNA sequence or its complement sequence and can also be a cDNA, or a region of DNA may be complementary to another region of DNA. The term “substantially complementary” as used herein means that two sequences hybridize under stringent hybridization conditions. The skilled artisan will understand that substantially complementary sequences need not hybridize along their entire length. In particular, substantially complementary sequences comprise a contiguous sequence of bases that do not hybridize to a target or marker sequence, positioned 3′ or 5′ to a contiguous sequence of bases that hybridize under stringent hybridization conditions to a target or marker sequence.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PC reaction, or the enzymatic cleavage of a polynucleotide by a ribozyme.

Examples of stringent hybridization conditions include incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

As used herein, the term “at least a portion” or “fragment” of a nucleic acid or polypeptide means a portion having the minimal size characteristics of such sequences, or any larger fragment of the full-length molecule, up to and including the full-length molecule. A fragment of a polynucleotide of the disclosure may encode a biologically active portion of a genetic regulatory element. A biologically active portion of a genetic regulatory element can be prepared by isolating a portion of one of the polynucleotides of the disclosure that comprises the genetic regulatory element and assessing activity as described herein. Similarly, a portion of a polypeptide may be 4 amino acids, 5 amino acids, 6 amino acids, 7 amino acids, and so on, going up to the full-length polypeptide. The length of the portion to be used will depend on the particular application. A portion of a nucleic acid useful as a hybridization probe may be as short as 12 nucleotides; in some embodiments, it is 20 nucleotides. A portion of a polypeptide useful as an epitope may be as short as 4 amino acids. A portion of a polypeptide that performs the function of the full-length polypeptide would generally be longer than 4 amino acids.

For PCR amplifications of the polynucleotides disclosed herein, oligonucleotide primers can be designed for use in PCR reactions to amplify corresponding DNA sequences from cDNA or genomic DNA extracted from any organism of interest. Methods for designing PCR primers and PCR cloning are generally known in the art and are disclosed in Sambrook et al. (2001) Molecular Cloning: A Laboratory Manual (3rd ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially mismatched primers, and the like.

Primers are short nucleic acids, for example DNA oligonucleotides at least about six nucleotides in length, and/or no longer than 10, 20, 50, 100 or 200 nucleotides in length, though in some embodiments they are longer. Primers may be annealed to a complementary target DNA strand by nucleic acid hybridization to form a hybrid between the primer and the target DNA strand, and then extended along the target DNA strand by a DNA polymerase enzyme. Primer pairs can be used for amplification of a nucleic acid sequence, e.g., by PCR or other nucleic acid amplification methods known in the art.

Methods for preparing and using probes and primers are described, for example, in Sambrook et al (Molecular Cloning: A Laboratory Manual, Cold Spring Harbor Laboratory Press, 1989), Ausubel et al., Current Protocols in Molecular Biology, Greene Publishing Associates and Wiley-Intersciences (1987), and Innis et al., PCR Protocols, A Guide to Methods and Applications, 1990, Innis et al (eds.), 21-27, Academic Press, Inc., San Diego, Calif. PCR primer pairs can be derived from a known sequence, for example, by using computer programs intended for that purpose, such as Primer (Version 0.5, © 1991, Whitehead Institute for Biomedical Research, Cambridge, Mass.), Primer3 (Version 2.6.1 © 2022, Whitehead Institute for Biomedical Research, Cambridge, Mass), and Benchling primer wizard (© 2019, Benchling).

Probes and primers comprise at least ten nucleotides of a nucleic acid sequence, although a shorter nucleic acid (e.g., six nucleotides) may be used as a probe or primer if it specifically hybridizes under stringent conditions with a target nucleic acid by methods well known in the art. One of ordinary skill in the art will appreciate that the specificity of a particular probe or primer increases with its length. Thus, for example, a primer comprising 20 consecutive nucleotides of a sequence will anneal to a target sequence (for instance, contained within a genomic DNA library) with a higher specificity than a corresponding primer of only 15 nucleotides. To enhance specificity, longer probes and primers can be used, for example probes and primers that comprise at least 20, 30, 40, 50, 60, 70, 80, 90, 100 or more consecutive nucleotides from any region of a target.

As used herein. “promoter” refers to a DNA sequence capable of controlling the expression of a coding sequence or functional RNA. The promoter sequence may consist of proximal and more distal upstream elements, the latter elements often referred to as enhancers. Accordingly, an “enhancer” is a DNA sequence that can stimulate promoter activity, and may be an innate element of the promoter or a heterologous element inserted to enhance the level or tissue specificity of a promoter.

As used herein, the term “expression”, as used herein, refers to the process by which a polypeptide or RNA molecule is produced based on the encoding sequence of a nucleic acid molecule, such as a gene. The process may include transcription, post-transcriptional control, post-transcriptional modification, translation, post-translational control, post-translational modification, or any combination thereof. An expressed nucleic acid molecule is typically operably linked to an expression control sequence (e.g., a promoter). Methods of expressing polypeptides and RNAs are known in the art.

The term “operably linked” means in this context the sequential arrangement of the promoter polynucleotide according to the disclosure with a further oligo or polynucleotide, resulting in transcription of said further polynucleotide. In some embodiments, the promoter sequences of the present disclosure are inserted just prior to a gene's 5′UTR, or open reading frame. In other embodiments, the operably linked promoter sequences and gene sequences of the present disclosure are separated by one or more linker nucleotides.

As used herein, the phrases “recombinant construct”, “expression construct”, “chimeric construct”, “construct”, “recombinant DNA construct” and “recombinant RNA” are used interchangeably herein. A recombinant construct comprises an artificial combination of nucleic acid fragments, e.g., regulatory and coding sequences that are not found together in nature. For example, a chimeric construct may comprise regulatory sequences and coding sequences that are derived from different sources, or regulatory sequences and coding sequences derived from the same source but arranged in a manner different than that found in nature. Such construct may be used by itself or may be used in conjunction with a vector. If a vector is used, then the choice of vector is dependent upon the method that will be used to transform host cells as is well known to those skilled in the art. For example, a plasmid vector can be used. The skilled artisan is well aware of the genetic elements that must be present on the vector in order to successfully transform, select and propagate host cells comprising any of the isolated nucleic acid fragments of the disclosure. The skilled artisan will also recognize that different independent transformation events will result in different levels and patterns of expression (Jones et al., (1985) EMBO J. 4:2411-2418; De Almeida et al., (1989) Mol. Gen. Genetics 218:78-86), and thus that multiple events must be screened in order to obtain lines displaying the desired expression level and pattern. Such screening may be accomplished by Southern analysis of DNA, Northern analysis of mRNA expression, immunoblotting analysis of protein expression, or phenotypic analysis, among others. Vectors can be plasmids, viruses, bacteriophages, pro-viruses, phagemids, transposons, artificial chromosomes, and the like, that replicate autonomously or can integrate into a chromosome of a host cell. A vector can also be a naked RNA polynucleotide, a naked DNA polynucleotide, polynucleotide composed of both DNA and RNA within the same strand, a poly-lysine-conjugated DNA or RNA, a peptide-conjugated DNA or RNA, a liposome-conjugated DNA, or the like, that is not autonomously replicating. As used herein, the term “expression” refers to the production of a functional end product e.g., an mRNA or a protein (precursor or mature).

A “vector” is a replicon, such as plasmid, phage, viral construct, cosmid, bacterial artificial chromosome, P-1 derived artificial chromosome, or yeast artificial chromosome to which another DNA segment may be attached. In some instances, a vector may be a chromosome such as in the case of an arm exchange from one endogenous chromosome engineered to comprise a recombination site to a synthetic chromosome. Vectors are used to transduce and express a DNA segment in a cell. A “bacterial artificial chromosome (BAC)” is a DNA construct capable of extrachromosomal replication and segregation in bacterial cells. A “yeast artificial chromosome (YAC)” is a DNA construct capable of extrachromosomal replication and segregation in yeast cells. A “mammalian synthetic chromosome (MAC)” refers to chromosomes that have an active mammalian centromere(s). A “human synthetic chromosome (HAC)” refers to a chromosome that includes a centromere that functions in human cells and that preferably is produced in human cells. For exemplary artificial chromosomes, see, e.g., U.S. Pat. Nos. 8,389,802; 7,521,240; 6,025,155; 6,077,697; 5,891,691; 5,869,294; 5,721,118; 5,712,134; 5,695,967; and 5,288,625.

As used herein, the term “expression vector” refers to a DNA construct containing a nucleic acid molecule that is operably linked to a suitable control sequence capable of affecting the expression of the nucleic acid molecule in a suitable host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a potential genomic insert. Once transformed into a suitable host, the vector may replicate and function independently of the host genome, or may, in some instances, integrate into the genome itself or deliver the polynucleotide contained in the vector into the genome without the vector sequence. An example of certain expression vectors is described, for example, in U.S. Pat. No. 11,390,882.

The term “CRISPR RNA” or “crRNA” refers to the guide RNA strand responsible for hybridizing with target DNA sequences and recruiting CRISPR endonucleases. crRNAs may be naturally occurring or may be synthesized according to any known method of producing RNA. The term crRNA and guide strand (gRNA) are equivalent and may be interchangeably used throughout this document.

The term “tracrRNA” refers to a small trans-encoded RNA. TracrRNA is complementary to and base pairs with crRNA to form a crRNA/tracrRNA hybrid, capable of recruiting CRISPR endonucleases to target sequences.

The term “guide sequence” or “spacer sequence” refers to the portion of a crRNA that is responsible for hybridizing with the target DNA.

The term “protospacer” refers to the DNA sequence targeted by a crRNA or sgRNA guide strand. In some embodiments the protospacer sequence hybridizes with the crRNA or sgRNA guide (spacer) sequence of a CRISPR complex.

The term “seed region” refers to the critical portion of a crRNA's or guide RNA's guide sequence that is most susceptible to mismatches with their targets. In some embodiments, a single mismatch in the seed region of a crRNA can render a CRISPR complex inactive at that binding site. In some embodiments, the seed regions for Cas9 endonucleases are located along that last 12 nucleotides of the 3′ portion of the guide sequence. In some embodiments, the seed regions for Cpf1 endonucleases are located along the first 5 nucleotides of the 5′ portion of the guide strand.

The term “Guide RNA” or “gRNA” as used herein refers to an RNA sequence or combination of sequences capable of recruiting a CRISPR endonuclease to a target sequence. Thus, as used herein, a guide RNA can be a natural or synthetic crRNA (e.g., for Cpf1), a natural or synthetic crRNA/tracrRNA hybrid (e.g., for Cas9), or a single-guide RNA (sgRNA).

The term “CRISPR landing site” as used herein, refers to a DNA sequence capable of being targeted by a CRISPR complex. Thus, in some embodiments, a CRISPR landing site comprises a proximately placed protospacer/Protospacer Adjacent Motif (PAM) combination sequence that is capable of being cleaved a CRISPR endonuclease complex.

The term “validated CRISPR landing site” refers to a CRISPR landing site for which there exists a guide RNA capable of inducing cleavage of said sequence. Thus, the term validated should be interpreted as meaning that the sequence has been previously shown to be cleavable by a CRISPR complex. Each “validated CRISPR landing site” will by definition confirm the existence of a tested guide RNA associated with the validation.

As used herein, the term “CRISPR complex” refers to a CRISPR endonuclease and guide RNA complex. The term CRISPR complex thus refers to a combination of CRISPR endonuclease and guide RNA capable of inducing a double stranded break at a CRISPR landing site.

As used herein, the term “directing sequence-specific binding” in the context of CRISPR complexes refers to a guide RNA's ability to recruit a CRISPR endonuclease to a CRISPR landing site.

As used herein, a “cassette” is a nucleic acid sequence, optionally encoding at least one selectable marker that can be inserted into the genome of a cell or into a plasmid or artificial chromosome, for instance a prokaryotic or eukaryotic cell. In some embodiments, the cassette is divided into one or more separate nucleic acid sequences.

As used herein, the terms “endonuclease” or “endonuclease enzyme” refers to a member or members of a classification of catalytic molecules that bind a recognition site encoded in a DNA molecule and cleave the DNA molecule at a precise location within or near the sequence.

As used herein, the terms “endonuclease recognition site”, recognition site”, “cognate sequence” or “cognate sequences” refer to the minimal string of nucleotides required for a restriction enzyme to bind and cleave a DNA molecule or gene.

### Embodiments of the Invention

This disclosure provides for methods of engineering synthetic chromosomes and/or synthetic genomes, and host cells comprising the synthetic chromosomes form other naturally occurring nucleic acid segments. In some embodiments, the methods described herein feature the use of certain cloning cassettes, cloning vectors, and an in vivo cut and assembly cloning methods to construct the synthetic chromosomes.

In some embodiments, a method for constructing a synthetic chromosome comprising steps of: providing host cells comprising an endogenous chromosome; transforming a cloning vector and a cloning cassette into the host cells; excising target genomic nucleic acids from the endogenous chromosome; recombining the excised target genomic nucleic acids with the cloning cassette via homologous recombination to form heterologous vectors comprising cloned sequences; extracting the heterologous vectors containing the cloned sequences from the host cells; digesting the heterologous vectors with a restriction endonuclease to release the cloned sequences from the heterologous vectors to provide released cloned sequences; and introducing the released cloned sequences, a centromere cassette, and a yeast artificial chromosomes or bacterial artificial chromosomes into a second host cell, wherein the released cloned sequences, the centromere cassette, and the yeast artificial chromosomes or bacterial artificial chromosomes recombine with one another via homologous recombination to produce the synthetic chromosome.

In some embodiments, the cloning cassette may comprise a single segment of nucleic acid or multiple segments of nucleic acid (e.g., 2 separate segments of nucleic acid). These segments may be free, that is, not part of a cloning vector. In other embodiments, the cloning cassette may be part of (i.e., integrated into) a cloning vector.

In some embodiments where the cloning cassette comprises two separate segments of nucleic acid, a first segment of nucleic acid comprises a first hook region and a second segment of nucleic acid comprises a second hook region. Each of the hook regions include adapter sequences configured to be complementary to another adapter sequence on a different cloned segment, a region of cloning vector, or a centromere cassette. Thus, each cloning cassette may have a different adapter sequence that specifies how these nucleic acid molecules can recombine with another nucleic acid molecule in the host cell. There is no limit to the number of sequences that may be used for the adapter regions. In some embodiments, each of the adapter regions is about 25 base pairs to about 400 base pairs in length, about 25 base pairs to about 350 base pairs in length, about 25 base pairs to about 300 base pairs in length, about 25 base pairs to about 250 base pairs in length, about 25 base pairs to about 200 base pairs in length, about 25 base pairs to about 150 base pairs in length, about 25 base pairs to about 100 base pairs in length, or about 25 base pairs to about 50 base pairs in length.

In some embodiments, each of the hook regions also includes a complementary region that is complementary to a region flanking the excised target genomic nucleic acids. Each complementary region is about 25 base pairs to about 200 base pairs in length, about 25 base pairs to about 175 base pairs in length, about 25 base pairs to about 150 base pairs in length, about 25 base pairs to about 125 base pairs in length, about 25 base pairs to about 100 base pairs in length, about 25 base pairs to about 75 base pairs in length, or about 25 base pairs to about 50 base pairs in length. In some embodiments, the complementary regions are about 5 base pairs to about 25 base pairs in length.

In some embodiments, each hook region includes an adapter sequence flanked by nucleic acid moieties comprising unique restriction endonuclease sites that may be used to excise the cloned sequences from a vector. In some embodiments, each of the unique restriction endonuclease site also may comprise a homology region. The homology regions are complementary to regions of the cloning vector to permit recombination into the cloning vector. In some embodiments, the unique restriction endonuclease site also may and homology region is about 4 base pairs to about 100 base pairs in length, about 4 base pairs to about 90 base pairs in length, about 4 base pairs to about 80 base pairs in length, about 4 base pairs to about 70 base pairs in length, about 4 base pairs to about 60 base pairs in length, about 4 base pairs to about 50 base pairs in length, about 4 base pairs to about 40 base pairs in length, about 4 base pairs to about 30 base pairs in length, or about 4 base pairs to about 20 base pairs in length. In some embodiments, where the cloning cassette is originally part of the cloning vector, then the homology regions may be omitted, and the unique restriction site may comprise a sequence of the shortest endonuclease recognition sequence.

In some embodiments, a hook region may comprise a unique restriction endonuclease site and a homology region that is complementary to regions of the cloning vector. The homology regions permit recombination into the cloning vector while the unique restriction endonuclease site may be used to excise the cloned sequences from a vector.

In some embodiments, where the cloning cassette comprises two separate segments of nucleic acid, a first segment of nucleic acid comprises a first hook region and a second segment of nucleic acid comprises a second hook region, wherein the first hook region comprises formula A-B-C, wherein: A comprises a unique restriction endonuclease site and first homology region comprising a nucleic acid sequence complementary to first region of the cloning vector; B comprises a first adapter region comprising a nucleic acid sequence complementary to another adapter region; and C comprises a first complementary region comprising a nucleic acid sequence complementary to a region flanking the excised target genomic nucleic acids; the second hook region comprises formula D-E-F, wherein: D comprises a second complementary region comprising a nucleic acid sequence complementary to a region flanking the excised target genomic nucleic acids; E comprises a second adapter region comprising a nucleic acid sequence complementary to another adapter region; and F comprises a unique restriction endonuclease site and a second homology region comprising a nucleic acid sequence complementary to second region of the cloning vector; and wherein the two separate pieces of nucleic acid are co-transformed into the host cells.

When the cloning cassette is part of the cloning vector, the cloning cassette may comprise a first hook region, a second hook region, and a multiple cloning site (MCS) disposed therebetween, wherein the first hook region comprises formula A-B-C, wherein: A comprises a unique restriction endonuclease site; B comprises a first adapter region comprising a nucleic acid sequence complementary to at least one other adapter region; and C comprises a first complementary region comprising a nucleic acid sequence complementary to a region flanking the one or more excised target genomic nucleic acids; the second hook region comprises formula D-E-F, wherein: D comprises a second complementary region comprising a nucleic acid sequence complementary to a region flanking the one or more excised target genomic nucleic acids; E comprises a second adapter region comprising a nucleic acid sequence complementary to one other adapter region; and F comprises a unique restriction endonuclease site; and wherein prior to the transformation step, the cloning cassette is linearized by restriction endonuclease digestion using a restriction endonuclease that recognizes and cuts a nucleic acid sequence within the MCS. In some embodiments, a cloning cassette need not include an adapter region if one or both complementary regions of the cloning cassette share homology with another complementary region of a separate cloning cassette or with a region of the cloning vector. Further, in some embodiments, the adapter regions can include naturally occurring sequences, synthetic sequences, or a combination of both naturally occurring sequences and synthetic sequences.

In some embodiments, the cloning cassette is about 100 to about 1000 base pairs in length, about 100 to about 900 base pairs in length, about 100 to about 800 base pairs in length, about 100 to about 700 base pairs in length, about 100 to about 600 base pairs in length, about 100 to about 500 base pairs in length, about 100 to about 400 base pairs in length, about 100 to about 300 base pairs in length, or about 100 to about 200 base pairs in length.

In some embodiments, after transforming a cloning vector and a cloning cassette into the host and then excising target genomic nucleic acids from the endogenous chromosome, the first complementary region and the second complementary region of the hook regions separately contact and binds to complementary nucleic acid sequences flanking the excised target genomic nucleic acids; and the first homology region and the second homology region separately contact and bind to a first region and a second region of the cloning vector, respectively.

In some embodiments, after the target genomic nucleic acid sequences are integrated into the cloning vector to form a heterologous vector comprising cloned sequences, a restriction endonuclease, in a digesting step, recognizes unique restriction endonuclease sites flanking the cloning cassette of the heterologous vector, thereby releasing the cloned sequences from the heterologous vectors to provide the released cloned sequences. In some embodiments, the released cloned sequences comprise the structure B-CS-E, wherein CS comprises the one or more cloned sequences.

In some embodiments, after being transformed into a second host cell, the released cloned sequences, the centromere cassette, and the YAC or BAC recombine with one another via homologous recombination to produce the synthetic chromosome, at least one of a first adapter region and a second adapter region of a first released clone sequence is complementary to and binds to at least one of the first adapter sequence and the second adapter sequence of a second released cloned sequence to permit homologous recombination between the first released clone sequence and the second released clone sequence to produce the synthetic chromosome.

In some embodiments, after being transformed into a second host cell or recipient cell, the released cloned sequences, the centromere cassette, and the YAC or BAC recombine with one another via homologous recombination to produce the synthetic chromosome. In some embodiments, at least one of a first adapter region and a second adapter region of a first released clone sequence is complementary to and binds to at least one of the first adapter sequence and the second adapter sequence of a second released cloned sequence to permit homologous recombination between the first released clone sequence and the second released clone sequence to produce the synthetic chromosome, and/or at least one of a first adapter region and a second adapter region of a released clone sequence is complementary to and binds to a complementary region or an adapter region of the centromere cassette or the YAC/BAC to permit homologous recombination between the released clone sequence and one or more of the centromere cassette and cloning vector to produce the synthetic chromosome.

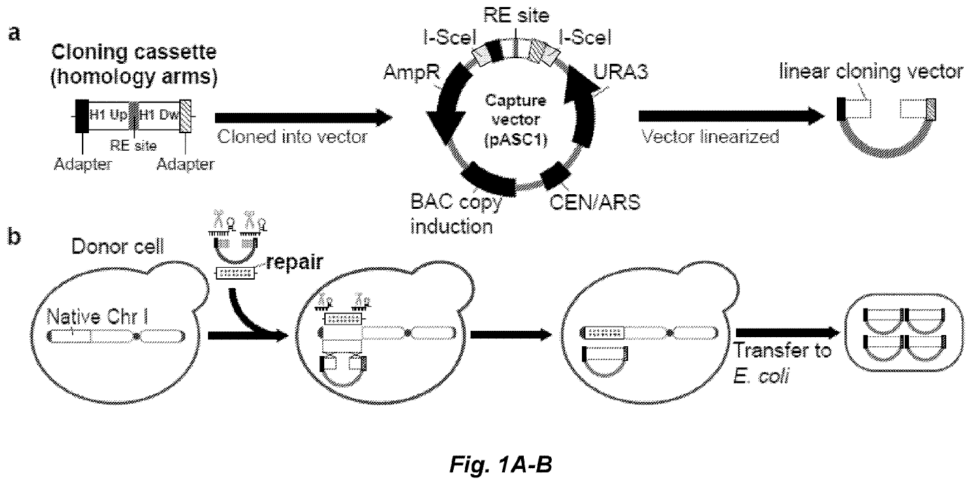

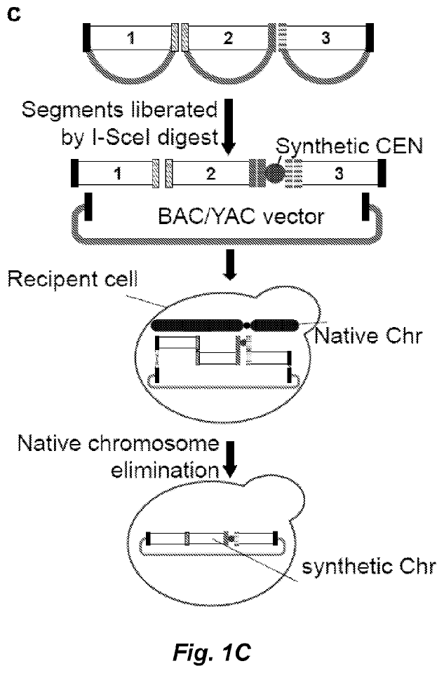

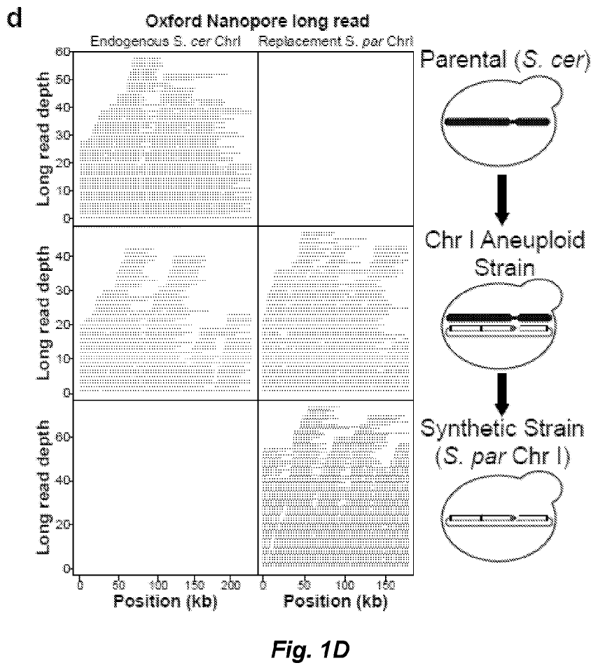

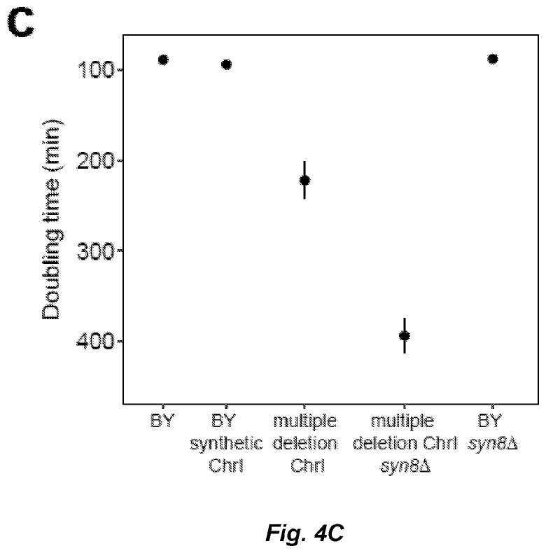

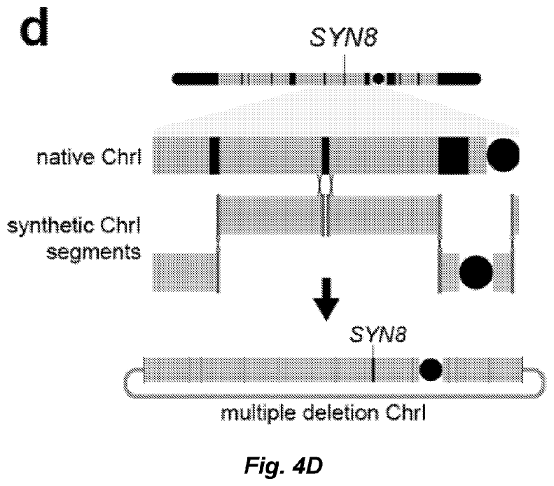

The methods described herein permit any number of target nucleic acid sequences to be excised from the host cell and inserted into a cloning cassette to form the cloned sequences. For example, the number of excised target nucleic acids can be 1, 2, 3, 4, 5, 6, 7, 8, 9, 10, 11, 12, 13, 14, 15, 16, 17, 18, 19, or 20 or more that can produce 1, 2, 3, 4, 5, 6, 7, 8, 9, 10, 11, 12, 13, 14, 15, 16, 17, 18, 19, or 20 or more cloned sequences. Once the resultant cloned sequences are introduced into the second host cell or recipient cell, 1, 2, 3, 4, 5, 6, 7, 8, 9, 10, 11, 12, 13, 14, 15, 16, 17, 18, 19, or 20 or more of the cloned sequences and a centromere cassette may recombine to form the synthetic chromosome (See, for example, FIG. 1 and FIG. 4).

In some embodiments, the host cells are cells of at least two different genera or species, and the synthetic chromosome is a chimeric chromosome comprising nucleic acid sequences from the two different genera or species. In some embodiments, the host cells are one or more of a yeast cell, a mammalian cell, or a bacterial cell. The second host cell can be any cell as disclosed herein and may be the same or different than the host cells.

In some embodiments, the different species are yeast species may comprise a Candida, Hansenula, Kluyveromyces, Pichia, Saccharomyces, Schizosaccharomyces, Yarrowia strain, Acremonium, Aspergillus, Aureobasidium, Cryptococcus, Filibasidium, Fusarium, Humicola, Magnaporthe, Mucor, Myceliophthora, Neocallimastix, Neurospora, Paecilomyces, Penicillium, Piromyces, Schizophyllum, Thermoascus, Thielavia, Tolypocladium, or Trichoderma strain. In other embodiments, the fungal cell is one or more of Aspergillus aculeatus, Aspergillus awamori, Aspergillus foetidus, Aspergillus japonicus, Humicola insolens, Humicola lanuginosa, Mucor miehei, Myceliophthora thermophila, Penicillium purpurogenum, Trichoderma harzianum, Trichoderma koningi, Trichoderma longibrachiatum, Trichoderma reesei, Trichoderma viride strain, Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Physcomitrella patens, and Neurospora crassa. Preferably, the host cells are one or more of Saccharomyces spp. such as Saccharomyces arboricolus, Saccharomyces bayanus, Saccharomyces bulderi, Saccharomyces cariocanus, Saccharomyces cariocus, Saccharomyces cerevisiae, Saccharomyces cerevisiae var. boulardii, Saccharomyces chevalieri, Saccharomyces dairenensis, Saccharomyces ellipsoideus, Saccharomyces eubayanus, Saccharomyces exiguus, Saccharomyces florentinus, Saccharomyces fragilis, Saccharomyces kudriavzevii, Saccharomyces martiniae, Saccharomyces mikatae, Saccharomyces monacensis, Saccharomyces norbensis, Saccharomyces paradoxus, Saccharomyces pastorianus, Saccharomyces spencerorum, Saccharomyces turicensis, Saccharomyces unisporus, Saccharomyces uvarum, Saccharomyces zonatus.

In certain embodiments, the mammalian cell is a murine cell. In certain embodiments, said mammalian cell is a bovine cell. In certain embodiments, said mammalian cell is a human cell. In certain embodiments, cells may be from other mammalian species including, but not limited to, equine, canine, porcine, ovine sources; or rodent species such as rat may be used. In some embodiments, the mammalian cell may be one of a retinal pigmented epithelial (RPE) cell, a hematopoietic cell, a red blood cell, a platelet, a pancreatic beta cell, a skin cell, a cardiomyocyte, a smooth muscle cell, an endothelial cell, a hepatocyte, a neuron, a glia cell, a skeletal muscle cell, or a vascular cell.

In some embodiments, the bacterial cell is one of Neisseria, Spirillum, Pasteurella, Brucella, Yersinia, Francisella, Haemophilus, Bordetella, Escherichia, Salmonella, Shigella, Klebsiella, Proteus, Vibrio, Pseudomonas, Bacteroides, Acetobacter, Aerobacter, Agrobacterium, Azotobacter, Spirilla, Serratia, Rhizobium, Chlamydia, Rickettsia, Treponema, Fusobacterium. Actinomyces, Bacillus, Clostridium, Corynebacterium, Erysipelothrix, Lactobacillus, Listeria, Mycobacterium, Myxococcus, Nocardia, Staphylococcus, Streptococcus, and Streptomyces.

The use of various host cells dictates the source of the cloned sequences (e.g., the excised target genomic nucleic acids), and ultimately, the composition of the synthetic chromosome. For example, one host cell may be a yeast cell, a second host cell may be a human cell, and a third host cell may be bacterial cell. Thus, there is no limit to the number of host cells that may be used, and each host cell may be a cell of a different genera from another host cell. Thus, in some embodiments, a host cell may comprise 1, 2, 3, 4, 5, 6, 7, 8, 9, 10, 11, 12, 13, 14, 15, 16, 17, 18, 19, or 20 or more different genera or species. If follows then that a synthetic chromosome may be made by introducing the clones sequences from any number of host cells into a recipient cell, such as a yeast cell, to construct the synthetic chromosome, resulting in a synthetic chromosome having, for example, 1, 2, 3, 4, 5, 6, 7, 8, 9, 10, 11, 12, 13, 14, 15, 16, 17, 18, 19, or 20 or more different sources of nucleic acid. Typically, the recipient cell or second host cell in which the synthetic chromosome is constructed through homologous recombination is a yeast cell such as a Saccharomyces spp.

In some embodiments, the host cells express, either constitutively or inducibly, a CRISPR endonuclease; and wherein the transformation step further comprises transforming one or more guide ribonucleic acids (gRNAs) into the one or more host cells that are complementary to regions flanking the one or more target genomic nucleic acid sequences of the endogenous chromosome, wherein the CRISPR endonuclease protein and the gRNAs excise the one or more target genomic nucleic acid sequences from the endogenous chromosome.

In some embodiments, the host cells express, either constitutively or inducibly, a Cas9 or Cas12a endonuclease protein; and wherein the transformation step further comprises transforming one or more guide ribonucleic acids (gRNAs) into the one or more host cells that are complementary to regions flanking the one or more target genomic nucleic acid sequences of the endogenous chromosome, wherein the Cas9 or Cas12a endonuclease protein and the gRNAs excise the one or more target genomic nucleic acid sequences from the endogenous chromosome.

In some embodiments, the host cell comprises a nucleic acid sequence encoding a CRISPR endonuclease that is integrated into the host cell chromosome. In other embodiments, the host cell includes a plasmid or other vector comprising a nucleic acid sequence encoding the CRISPR endonucleases. In still other embodiments, the host cell comprises one or more plasmids or other vectors comprising nucleic acid sequences that encode both the CRISPR endonucleases and one or more gRNAs.

Other known CRISPR endonucleases include, but are not limited to, Cas8a, Cas5, Cas8b, Cas8c, Cas10d, Cse1, Cse2, Csy1, Csy2, Csy3, Cas10, Csm2, Cmr5, Cas10, Csx11, Csx10, Csf1, Cas9, Csn2, Cas4, Cas12, Cas12a (Cpf1), Cas12b (C2c1), Cas12c (C2c3), Cas12d (CasY), Cas12e (CasX), Cas12f (Cas14, C2c10), Cas12g, Cas12h, Cas12i, Cas12k (C2c5), C2c4, C2c8, and C2c9. In some embodiments, the CRISPR endonuclease is Cas9 or Cas12a. The CRISPR endonuclease should be a nuclease targeting deoxyribose nucleic acid. These enzymes are known; for example, the amino acid sequence of S. pyogenes Cas9 protein may be found in the SwissProt database under accession number Q99ZW2.

The CRISPR/Cas9 system is used to treat target genomic nucleic acid in order to generate double-stranded breaks near the target genomic nucleic acid. CRISPR (Clustered Regularly Interspaced Short Palindromic Repeats) and CRISPR-associated (Cas) endonucleases were originally discovered as adaptive immunity systems evolved by bacteria and archaea to protect against viral and plasmid invasion. Naturally occurring CRISPR/Cas systems in bacteria are composed of one or more Cas genes and one or more CRISPR arrays consisting of short palindromic repeats of base sequences separated by genome-targeting sequences acquired from previously encountered viruses and plasmids (called spacers). (Wiedenheft et al. Nature. 2012; 482:331; Bhaya et al., Annu. Rev. Genet. 2011; 45:231; and Terms et al., Curr. Opin. Microbiol. 2011: 14:321). Bacteria and archaea possessing one or more CRISPR loci, respond to viral or plasmid challenge by integrating short fragments of foreign sequence (protospacers) into the host chromosome at the proximal end of the CRISPR array. Transcription of CRISPR loci generates a library of CRISPR-derived RNAs (crRNAs) containing sequences complementary to previously encountered invading nucleic acids (Haurwitz et. al., Science. 2012:329; 1355; Gesner et. al., Nat. Struct. Mol. Biol. 2001:18:688; Jinek et. al., Science. 2012:337; 816-21). Target recognition by crRNAs occurs through complementary base pairing with target DNA, which directs cleavage of foreign sequences by means of Cas proteins.

There are at least five main CRISPR system types (Type I, II, III, IV and V) and at least 16 distinct subtypes (Makarova et al., Nat Rev Microbiol. 2015. Nat. Rev. Microbiol. 13, 722-736; Makarova et al., Nature Reviews. Microbiology. 18 (2): 67-83). CRISPR systems are also classified based on their effector proteins. Class 1 systems possess multi-subunit crRNA-effector complexes, whereas in class 2 systems all functions of the effector complex are carried out by a single protein (e.g., Cas9 or Cas12a/Cpf1). In some embodiments, the present disclosure teaches using type II and/or type V single-subunit effector systems. Thus, in some embodiments, the present disclosure teaches the use of class 2 CRISPR systems. In some embodiments, the CRISPR system used to form the synthetic chromosomes described herein comprises the Cas12a/Cpf1 system (Bijoya et al., Biomedical Journal, Vol. 1, Issue 1, 8-17, (2020)).

In some embodiments, the present disclosure teaches methods of gene editing using a Type 11 CRISPR system. In some embodiments, the present disclosure teaches Cas9 Type 11 CRISPR systems. Type II systems rely on a i) single endonuclease protein, ii) a transactivating crRNA (tracrRNA), and iii) a crRNA where a 20-nucleotide (nt) portion of the 5′ end of crRNA is complementary to a target nucleic acid. The region of a CRISPR crRNA strand that is complementary to its target DNA protospacer is hereby referred to as “guide sequence.”

Cas9 endonucleases produce blunt end DNA breaks and are recruited to target DNA by a combination of a crRNA and a tracrRNA oligos, which tether the endonuclease via complementary hybridization of the RNA complex. In some embodiments, the crRNA and a tracrRNA are combined into a single gRNA.

In some embodiments, DNA recognition by the crRNA/endonuclease complex requires additional complementary base pairing with a protospacer adjacent motif (PAM) (e.g., 5′-NGG-3′) located in a 3′ portion of the target DNA, downstream from the target protospacer. (Jinek et. al., Science. 2012:337; 816-821). In some embodiments, the PAM motif recognized by a Cas9 varies for different Cas9 proteins. An exemplary use of a CRISPR system is described in U.S. Pat. App. No. US 2019/0134227.

In some embodiments, one or more repair templates are transformed into the host cell to permit repair of the endogenous chromosome after excision of the target genomic nucleic acids. For example, the one or more repair templates may comprise one or more selectable markers; a first homology arm; and a second homology arm; wherein the first homology arm and the second homology are complementary to nucleic acid sequences of the endogenous chromosome that flank the one or more target genomic nucleic acids to permit homologous recombination between the one or more repair templates and exogenous chromosome, thereby repairing the endogenous chromosome.

Embodiments of the methods disclosed herein permit the formation of certain synthetic chromosomes that comprises one more deletions of segments of nucleic acid as compared to the endogenous chromosome, or, in other embodiments, permits formation of synthetic chromosome comprising a rearrangement of one or more nucleic acid segments as compared to an arrangement of corresponding nucleic acid segments of the endogenous chromosome. In other embodiments, recombinant cells may be produced with a synthetic chromosome made using a method as disclosed herein.

In some embodiments, the cloned segments are propagated in library of cloned segments in cloning vectors or heterologous vectors. The library may be contained in, for example, bacterial cells (e.g., E. coli) for ease of use and manipulation. In some embodiments, the cloned segment of genomic nucleic acid is about 100 kB, about 95 kB, about 90 kB, about 85 kB, about 80 kB, about 75 kB, about 70 kB, about 65 kB, about 60 kB, about 55 kB, about 50 kB, about 45 kB, about 40 kB, about 35 kB, about 30 kB, about 25 kB, about 20 kB, about 15 kB, about 14 kB, about 13 kB, about 12 kB, about 11 kB, about 10 kB, about 9 kB, about 8 kB, about 7 kB, about 6 kB, about 5 kB, about 4 kB, about 3 kB, about 2 kB, about 1 kB, or about 0.5 kB. In some embodiments, the cloned sequences exclude the telomeric sequences or sub-telomeric sequences of nucleic acid. In other embodiments, the cloned sequences include the telomeric sequences or sub-telomeric sequences of nucleic acid. In other embodiments, the deleted segments of genomic nucleic acid are about 100 kB, about 95 kB, about 90 kB, about 85 kB, about 80 kB, about 75 kB, about 70 kB, about 65 kB, about 60 kB, about 55 kB, about 50 kB, about 45 kB, about 40 kB, about 35 kB, about 30 kB, about 25 kB, about 20 kB, about 15 kB, about 14 kB, about 13 kB, about 12 kB, about 11 kB, about 10 kB, about 9 kB, about 8 kB, about 7 kB, about 6 kB, about 5 kB, about 4 kB, about 3 kB, about 2 kB, about 1 kB, or about 0.5 kB.

In some embodiments, the synthetic chromosome includes a centromere cassette comprising an origin or replication. Origins of replication are regions of DNA from which DNA replication during the S phase of the cell cycle is primed. While yeast origins of replication, termed autonomously replicating sequence (ARS), are fully defined (Theis et al., Proc. Natl. Acad Sci. USA 94: 10786-10791, 1997), there does not appear to be a specific corresponding origin of replication sequence in mammalian DNA (Grimes and Cooke, Human Molecular Genetics, 7(10): 1635-1640, 1998). There are, however, numerous regions of mammalian DNA that can function as origins of replication (Schlessinger and Nagaraja, Ann. Med., 30:186-191, 1998; Dobbs et al., Nucleic Acids Res. 22:2479-89, 1994; and Aguinaga et al., Genomics 5:605-11, 1989). It is known that for every 100 kb of mammalian DNA sequence there is a sequence that will support replication, but in practice sequences as short as 20 kb can support replication on episomal vectors (Calos, Trends Genet. 12:463-466, 1996). This indicates that epigenetic mechanisms, such as CpG methylation patterning likely play some role in replication of DNA (Rein et al., Mol. Cell. Biol. 17:416-426, 1997).

In some embodiments, the origin of replication of a disclosed synthetic chromosome (SC) can be any size that supports replication of the SC. One way of ensuring that the SC has a functional on sequence is to require that SC contain at least 5 kb of genomic DNA. In other embodiments, it contains at least 10 kb, 15 kb, 20 kb, 25 kb, 30 kb, 35 kb, 40 kb, 45 kb, 50 kb, 60 kb, 70 kb, 80 kb, 90 kb, or 100 kb of genomic DNA. In general, any region of DNA could be used as origin of replication. If there is replication of the SC, the origin of replication is functioning as desired.

The origin of replication of the SC can be obtained from any number of sources, including particularly any number of sources of eukaryotic DNA. By way of example, it can be any region of eukaryotic DNA that is not based on a repeat sequence, such as the alphoid DNA sequence. A native alphoid DNA sequence does not contain an origin of replication in it, because the repeat sequences are so small, for example about 170 base pairs, and can be repeated many times, so that there is not enough variation for an origin of replication sequences to be present. However, in many instances these regions, when they contain multiple alphoid DNA repeats, can function as origins of replication in eukaryotes, such as human, cells (see, e.g., U.S. patent publication No. 2004/0245317). Also included in the SCs as described herein is a centromere region. It is understood that a centromere region, broadly defines a functional stretch of nucleic acid that allows for segregation of the SC during the cell cycle and during mitosis. Thus, in some embodiments, a centromere cassette comprises an origin of replication, a centromere sequence, and an adapter arm flanking both the origin of replication and the centromere sequence, wherein the adapter arm is complementary to at least one other adapter region of the cloning cassette. Optionally, the centromere cassette also may include one or more selectable markers.

Various cloning vectors for use with embodiments of the invention are known in the art. Preferred cloning vectors include Bacterial artificial chromosomes (BACs) which are DNA constructs capable of accommodating larger inserts than plasmids. BACs have an advantage of being able to capture large genomic segments up to approximately 350 kilobases, thereby facilitating the cloning of entire genes and other nucleic acid sequences including noncoding regions and regulatory elements. Hallmark features of BACs include, but are not limited to, a P1 or F origin of replication; an antibiotic resistance gene which may be one of several genes conferring resistance to kanamycin, spectinomycin, streptomycin, ampicillin, carbenicillin, bleomycin, erythromycin, polymyxin B, tetracycline, or chloramphenicol, among others; a parA and/or parB sequence for partitioning F plasmid DNA to daughter cells during cell division; and a polylinker region. A suitable BAC vector backbone may accommodate an entire gene or other large locus, or a library of inserts generated from a fractionated genome.

Similar to BACs, yeast artificial chromosomes (YACs) may be used to accommodate relatively large payloads of polynucleotides. In some implementations, YACs may be preferable to BACs for the cloning of polynucleotides into a cell. Hallmark features of YACs may include, but are not limited to, an ARS sequence (The ARS feature functions similarly to the bacterial origin of replication and allows for independent propagation of the plasmid in cells) to allow the YAC to replicate autonomously and extra chromosomally; CEN sequences (The CEN sequence is the attachment point for kinetochore complexes and allows for faithful segregation of copies of the plasmid to daughter cells during mitosis) to confer mitotic stability and allow for faithful segregation and maintenance of copy number during cell division; a selectable marker which may include, but is not limited to, URA3, LEU2, HIS3, TRP1, ADE2, LYS2, or MET15, an autoselection system URA3, FBAI, POT/TPI, or CDCx, or a dominant selectable marker gene may including, neomycin phosphotransferase (kan), hygromycin B phosphotransferase gene (hph), nourseothricin N-acetyltransferase (nat), phosphinothricin acetyltransferase (pat), phleomycin resistance (ble), acetamidase gene of Aspergillus nidulans (amdSYM), or thymidine kinase (tk). YACs may accommodate relatively large polynucleotide insert sizes up to 3000 kb in length (Dunnen, et al., Hum Mol Genet, 1(1):19-28 (1992)). Various BACS and YACS are described, for example, in U.S. Pat. No. 11,583,556, 10,99,542.

Thus, in some embodiments, the one or more yeast artificial chromosomes or bacterial artificial chromosomes comprises one or more selectable markers, and one or more homology arms that are complementary to one or more adapter regions of the hook regions of the cloning cassette.

In some embodiments, the one or more cloning vectors are BACs/YACs, and may comprise one or more selectable markers, an origin of replication, a centromere sequence, one or more sop genes, an oriV sequence, an oriS sequence, one or more rep genes, or a combination thereof. Nucleic acids can be introduced into a host cell via any known means such as, but not limited to, conjugation, transformation, transduction, electroporation, or a combination thereof.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### Example 1

### Example 1. CReATiNG Synthetic Chromosomes from Natural DNA

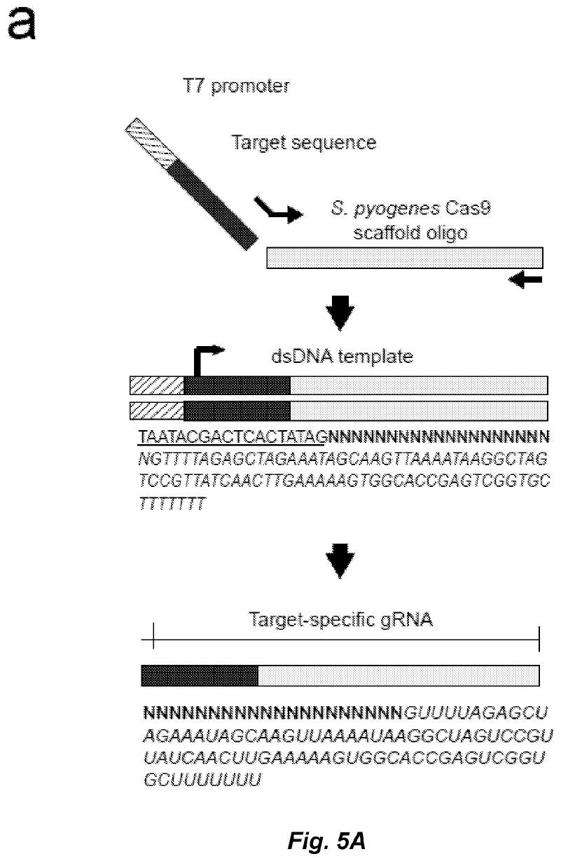

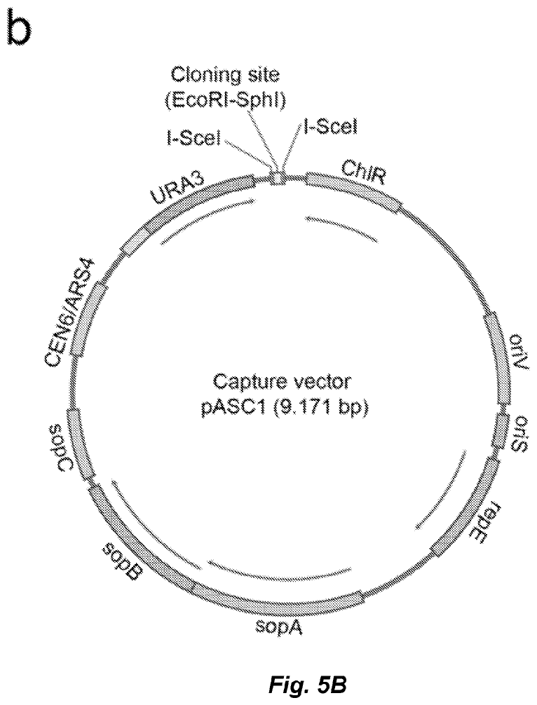

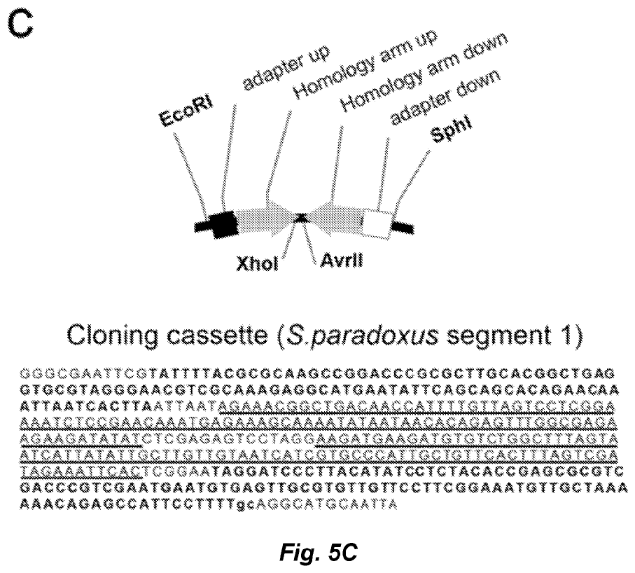

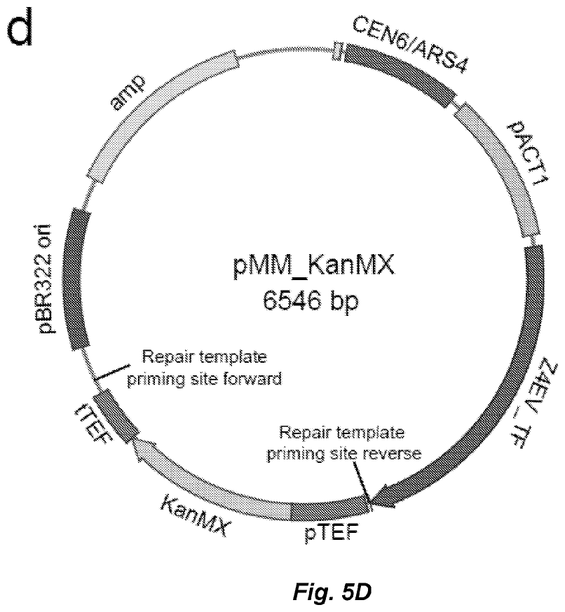

A system for in vivo cloning and reprogramming of natural DNA. CReATiNG involves cloning segments of natural chromosomes in yeast donor cells and then programmably assembling these segments into synthetic chromosomes in different recipient cells. To clone a target segment, we co-transform three reagents into cells that constitutively express Cas9 (FIGS. 1A and B; FIG. 5; Table 1): 1) in vitro transcribed guide RNAs (gRNAs) that direct Cas9 to cut a segment on each side, excising it from a chromosome; 2) a linear Bacterial Artificial Chromosome/Yeast Artificial Chromosome (BAC/YAC) cloning vector flanked by homology to the ends of a segment, enabling capture of a segment in vivo by homologous recombination; and 3) a repair template comprised of a dominant drug marker flanked with homology arms that allow a cell to reconstitute its broken chromosome, resulting in replacement of a segment with a marker.

Prior to cloning a given target segment, we modify the cloning vector by introducing a −500 bp cloning cassette that is synthesized de novo. A cloning cassette contains segment-specific homology arms separated by restriction sites and bounded by I-SceI sites, which are absent from the yeast nuclear genome. Between the I-SceI sites and homology arms, we also include 100 bp DNA sequences (adapters) that are not present in the S. cerevisiae genome. These adapters are used to program how segments will assemble later, as different segments with the same adapters will recombine in vivo. After transformation with the cloning reagents, cells containing successful cloning events can be isolated by selection on the markers in the cloning vector and the repair template. Cloned segments can then be extracted from yeast donor cells, transformed into E. coli for amplification, and extracted from E. coli for assembly in yeast recipient cells.

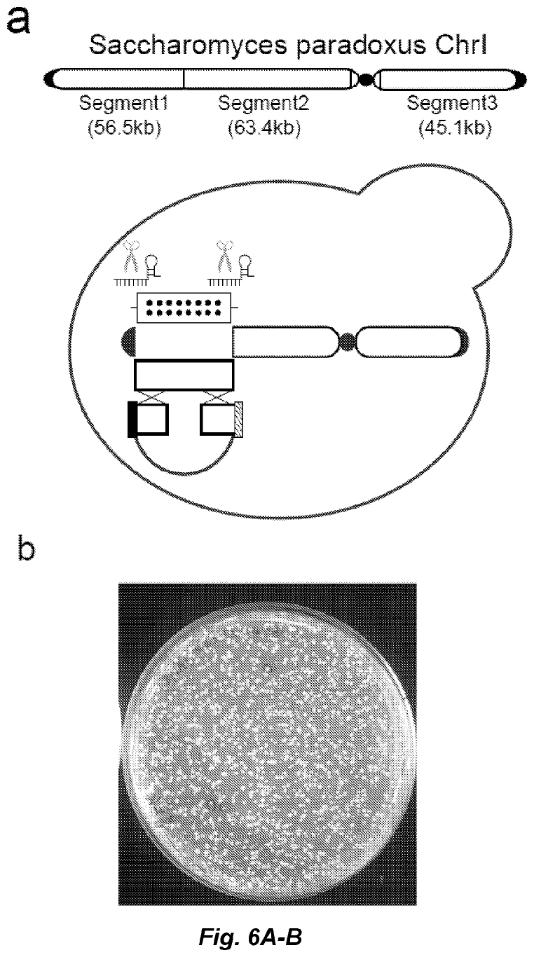

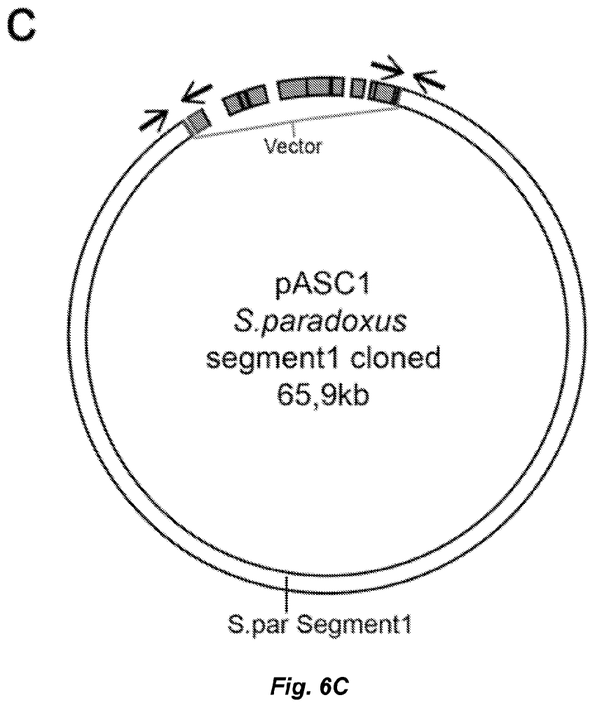

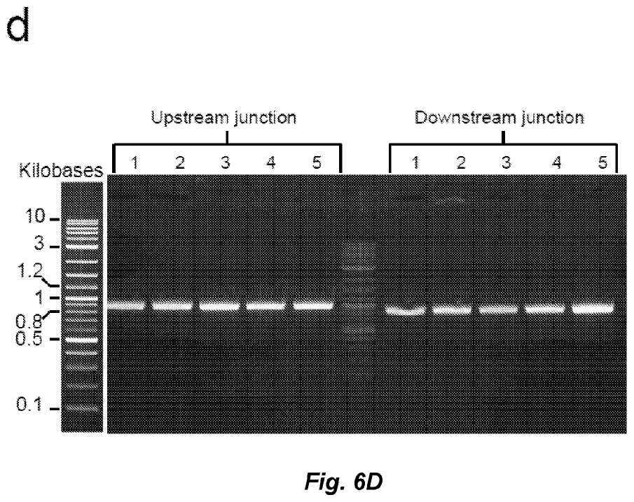

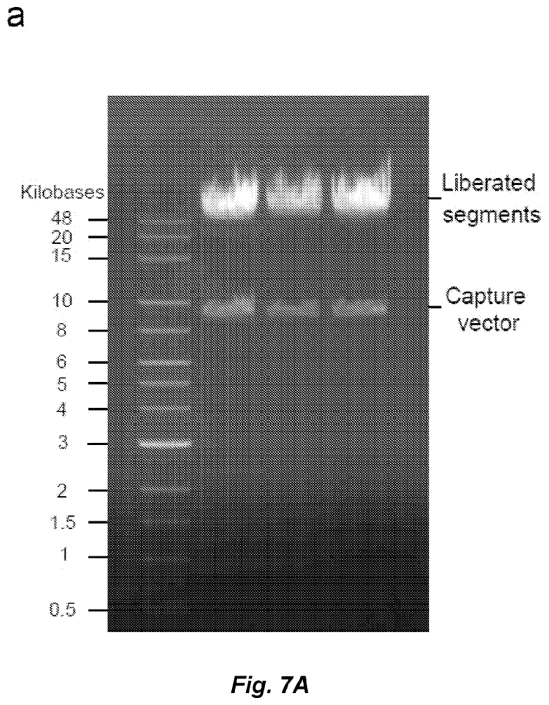

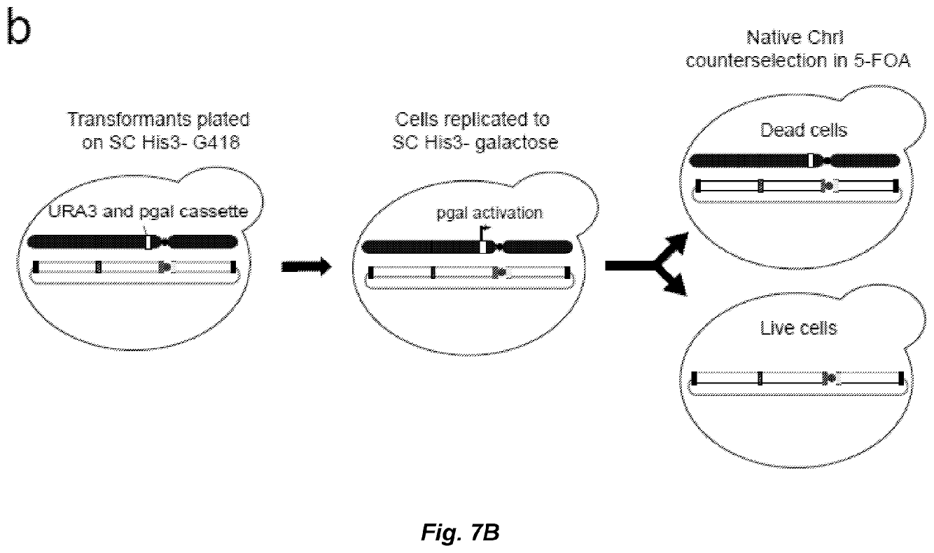

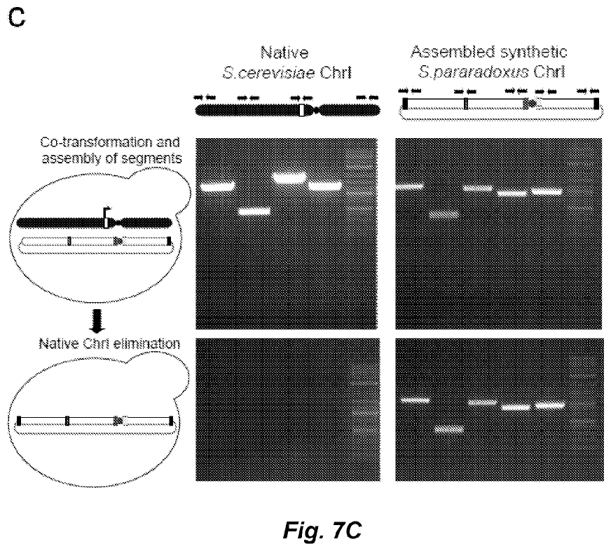

Initial assembly of a chromosome using CReATiNG. To prototype CReATiNG, we used Chromosome I (ChrI), a 230 kb chromosome containing 117 known or predicted protein-coding genes. ChrI is the smallest chromosome in the Saccharomyces genus and shows synteny between species. In silico, we divided S. paradoxus ChrI into three non-overlapping segments between 51 and 64 kb, which contained the entire chromosome except the centromere, subtelomeres, and telomeres (FIG. 6A). To assemble segments in their natural order and orientations, we used distinct adapters to specify the junctions between segments 1 and 2, segment 2 and the centromere cassette, and the centromere cassette and segment 3. We excluded subtelomeres from all subsequent work, as they are completely dispensable and highly variable across Saccharomyces strains and species. Telomeres were excluded because they are not amenable to cloning and we assemble chromosomes as circular, rather than linear, molecules. To enable cloning of the S. paradoxus ChrI segments, we generated a Cas9-expressing version of the S. paradoxus CBS5829 strain. We then performed three transformations, each targeting a different segment (Table 2 and 3). For each transformation, we checked five random colonies by amplifying each junction between a cloned segment and the vector (FIGS. 6B-D; Table 4). 14 of 15 (93%) colonies showed successful cloning based on amplification of both junctions (Table 5). After transfer to and amplification in E. coli, the three S. paradoxus ChrI segments were extracted, liberated from the vector by I-SceI digestion, and purified (FIG. 7A).

Next, we assembled the segments into a circular chromosome by co-transforming them, the centromere cassette with appropriate adapters, and a centromere-free BAC/YAC into the BY4742 (BY) reference strain of S. cerevisiae (FIG. 1C). Following transformation, we selected recipient cells in which the five molecules had assembled. Of five colonies checked by PCR of junctions between assembled segments, four (80%) contained the complete assembly (Table 6). We then performed whole genome Oxford Nanopore sequencing of a single yeast clone and confirmed the presence of two copies of ChrI, one from BY and one from S. paradoxus (FIG. 1D).

To produce a euploid strain containing only S. paradoxus ChrI, we conditionally destabilized and selected against BY ChrI (FIG. 7B). Chromosome elimination involves disrupting centromere function with an inducible GAL1 promoter and counter selecting a URA3 marker on the chromosome using 5-FOA. We verified complete elimination of BY ChrI by PCR (FIG. 7C) and Oxford Nanopore Technologies sequencing (FIG. 1D). We also used the Oxford Nanopore data to confirm that the euploid strain with synthetic S. paradoxus ChrI had the expected sequence and structure genome-wide, with the exception of a single point mutation in a non-functional portion of the centromere cassette.

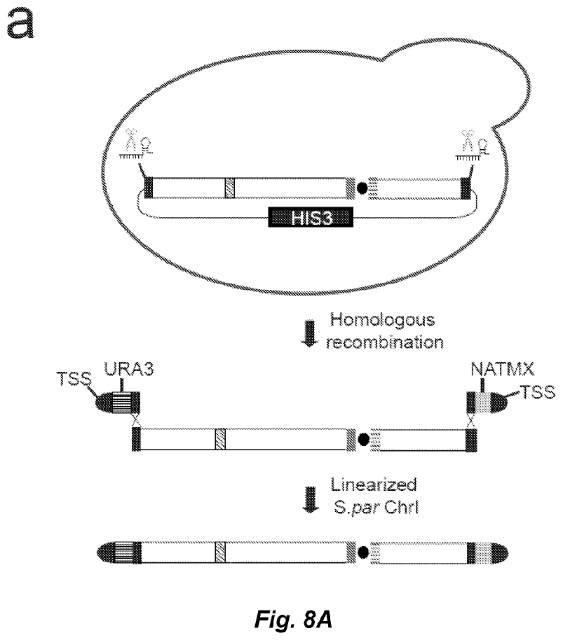

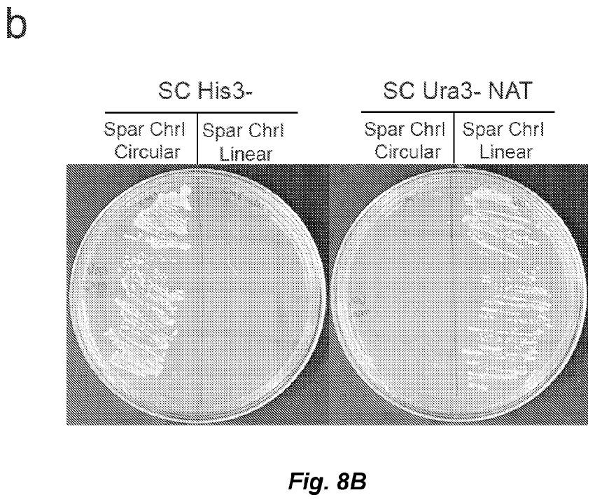

While the remaining work in this manuscript was conducted with circular chromosomes, some chromosome synthesis applications could require linear chromosomes. To confirm that it is possible to convert chromosomes synthesized by CReATiNG from circular to linear forms, we linearized the synthetic S. paradoxus ChrI. We used Cas9 to introduce double-strand breaks near each junction between the chromosome and the cloning vector. In addition to gRNAs, we co-transformed repair templates for both chromosome ends, each of which contained a synthetic telomere seed (Murray et al., Nature 305, 189-193 (1983)) and a distinct selectable marker. By selecting for the markers in both repair templates, we obtained cells containing a linear S. paradoxus ChrI (FIG. 8).

Recombination of chromosomes between strains and species. After confirming that CReATiNG can be used to build synthetic chromosomes that replace the native chromosomes in recipient cells, we explored potential applications. The first application was to synthetically recombine chromosomes between strains and species, which could aid efforts to study the genetic basis of heritable phenotypes. Relative to the crosses conventionally used to generate recombinants, the advantages of CReATiNG are that it does not require mating, meiosis, or natural synteny. Additionally, CReATiNG allows three or more parental chromosomes to recombine in a single assembly. The main constraint of CReATiNG for synthetically recombining chromosomes is that at present it cannot be applied genome wide.

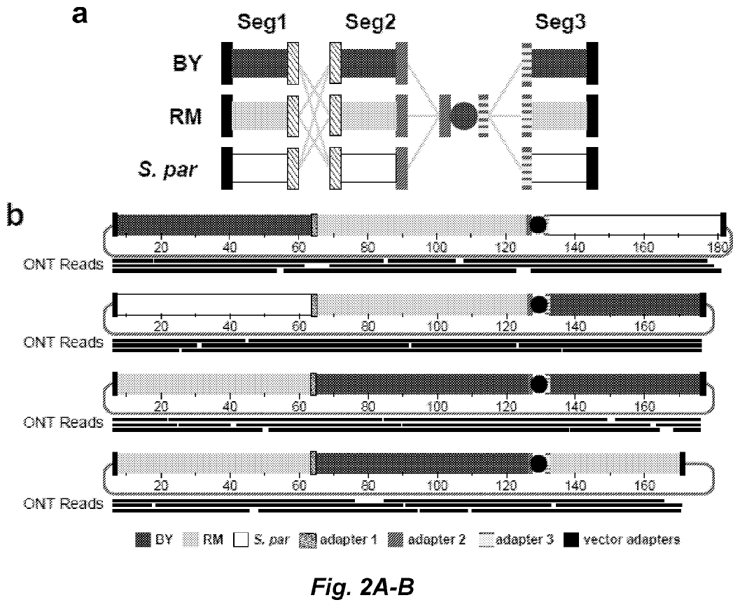

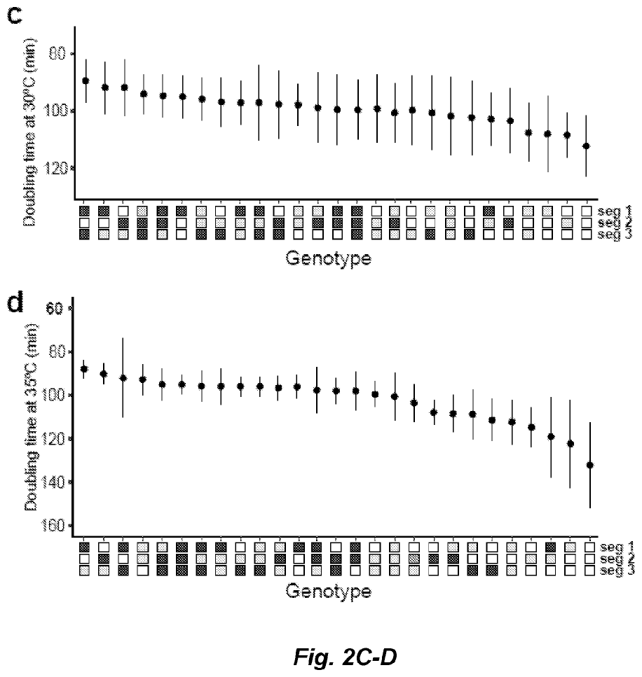

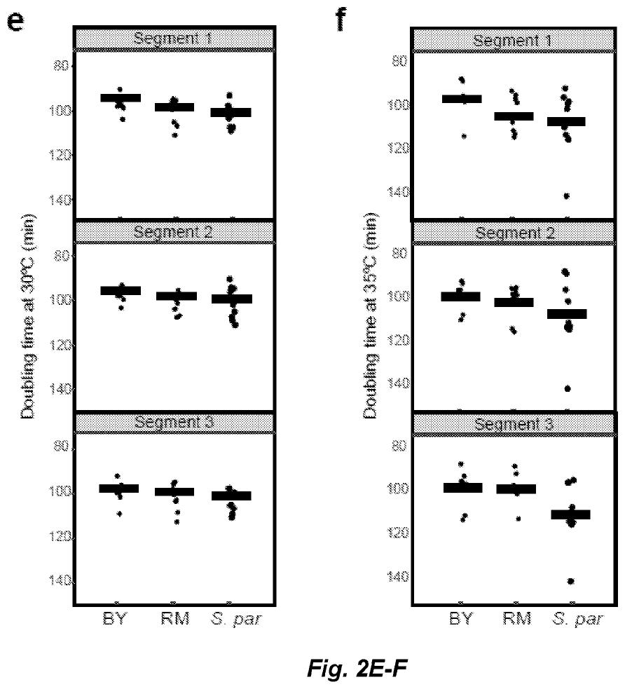

To use CReATiNG to recombine chromosomes synthetically, we next cloned the three ChrI segments from BY and another S. cerevisiae strain, the vineyard isolate RM11-1a (RM). During cloning, we appended the same adapters that were used for S. paradoxus segments, making it possible to generate all-possible syntenic combinations of the BY, RM, and S. paradoxus segments (FIG. 2A). As we had already generated a strain containing an entirely S. paradoxus ChrI, we individually assembled the 26 remaining possible chromosomes. These assemblies had efficiencies between 20 and 100% based on PCR examination of five colonies per transformation (FIG. 2B; Table 6). After elimination of native ChrI, a single successful assembly per design was then confirmed by Oxford Nanopore Technologies sequencing. In a few cases, cells with correct assemblies also possessed additional partial assemblies, which were eliminated by multiple rounds of streaking for single colonies followed by PCR confirmation of loss.

Next, we measured the growth rates of these strains with recombinant chromosomes in two conditions-rich liquid medium at 30 and 35° C. (FIGS. 2C and D; Table 7). S. paradoxus is known to be more sensitive to high temperature than S. cerevisiae. This phenotypic difference was also present in the donor strains. Among the 27 strains with recombinant chromosomes, growth rates varied substantially in both conditions (one-way ANOVAs, p-values≤6.6×10⁻¹¹). In addition, the strain carrying a fully S. paradoxus ChrI exhibited the slowest growth at both temperatures, but the difference between it and other strains was more severe at higher temperature. The results corroborate a recent finding that genetic differences on ChrI contribute to variation in thermotolerance between the two species.

The 27 strains with recombinant chromosomes also provide an opportunity to measure the individual and combined phenotypic effects of the three ChrI segments. We found that two segments had significant effects on growth at 30° C. (one-way ANOVAs, segment 1 p-value=0.008 and segment 2 p-value=0.019). However, at 35° C., all three segments had highly significant effects (one-way ANOVAs, p-values≤2.8×10⁻⁵; FIGS. 2E and F). These results show that all three ChrI segments contribute to growth variation across temperatures, with segment 3 in particular showing a strong interaction with temperature. We also used the data to measure epistasis among the segments and detected significant pairwise and three-way genetic interactions (F tests, p-values≤3.6×10⁻⁴; Table 8). Thus, CReATiNG is a useful tool for studying the contribution of genetic factors, including genotype-by-environment interactions and epistasis, to trait differences between strains and species.

Chromosome restructuring. CReATiNG can also be used to experimentally probe the structural rules underpinning chromosome organization, a topic relevant to genome function and evolution. Recent work suggests yeast can tolerate a diversity of chromosome structures, but most of these studies preserved the order of naturally linked genes³⁰⁻³³. CReATiNG makes it possible to restructure the contents of a chromosome in specific non-natural configurations that are programmed using adapters. CReATiNG can be used to synthesize chromosomes with one or more inversions, duplications, deletions, or modifications to gene order.

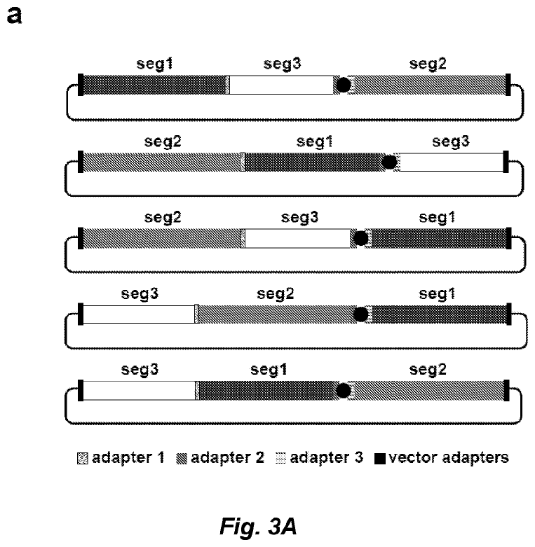

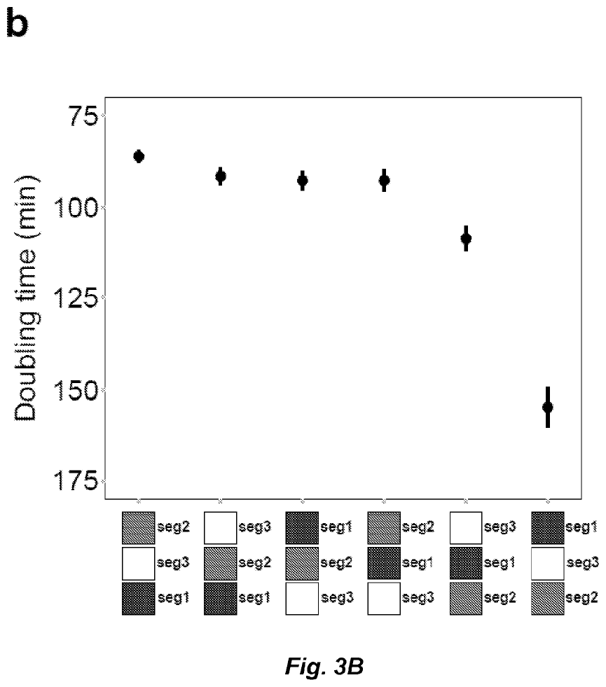

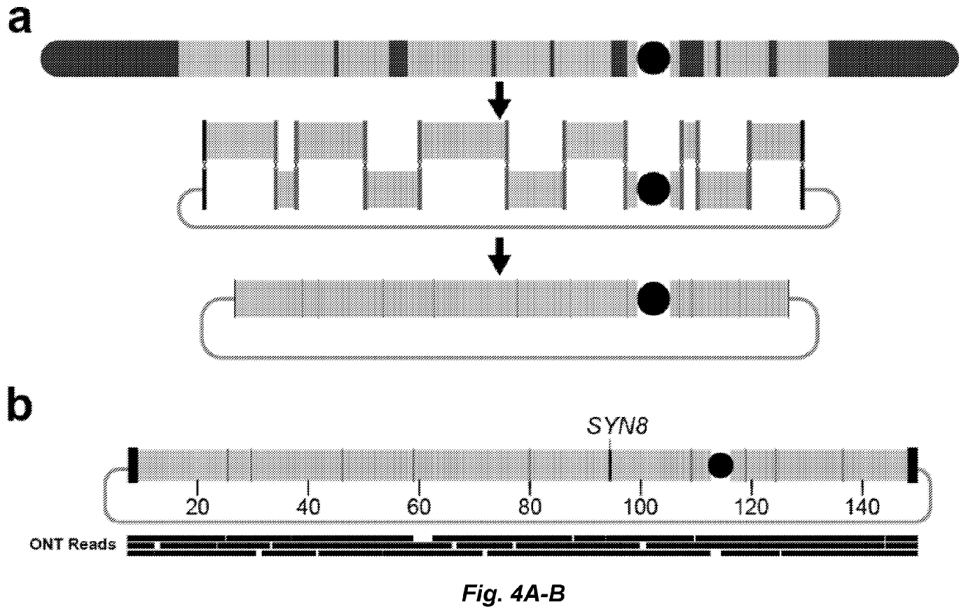

To demonstrate how CReATiNG can be used in chromosome restructuring, we re-cloned segments 1 through 3 from BY. During this round of cloning, we modified the adapters appended to each segment, making it possible to assemble the segments in all possible orders without inverting any segment. Using these re-cloned segments, we designed five non-natural ChrI structures with the same content but different orders (i.e., 1-3-2, 2-3-1, 2-1-3, 3-1-2, and 3-2-1) (FIG. 3A). We produced euploid strains with each non-natural ChrI structure by assembling segments with appropriate adapters and then eliminating the native ChrI. Each assembly was verified by junction PCRs and Oxford Nanopore sequencing (FIG. 3A; Table 9).

While all five strains possessing restructured versions of ChrI were viable, they also showed substantial phenotypic variation. The 2-3-1 configuration exhibited a 7% growth improvement relative to the natural 1-2-3 configuration, which had been generated earlier in the work on synthetic recombinants FIG. 3B; Table 10). By contrast, the 3-1-2 and 1-3-2 configurations respectively showed growth reductions of 18% and 68% relative to the natural 1-2-3 configuration. This result shows that relocating segment 2 to the natural position of segment 3 significantly impedes growth. However, the degree of this impairment also depends on the locations of segments 1 and 3. These findings show how programmably restructuring chromosomes with CReATiNG can be used to identify non-natural chromosome configurations with phenotypic effects.

Multiplex gene deletion and chromosome streamlining. Another application of CReATiNG is highly multiplexed deletion, a task that remains challenging for conventional genome editing technologies. Multiplexed deletion could enable the generation of streamlined chromosomes in which many non-essential genetic elements have been eliminated. Such streamlining can facilitate the production of yeast strains with a substantially reduced gene complement, as well as the reorganization of functionally related genes into modules. CReATiNG simplifies multiplexed deletion: segments of a natural chromosome that should be retained can be cloned and assembled, cleanly deleting all intervening parts of a natural chromosome.

We designed a CReATiNG workflow to delete 10 regions of ChrI, summing to 30 kb and containing 18 non-adjacent genetic elements in aggregate (12 protein-coding genes, 2 tRNAs, 3 LTRs, and 1 LTR retrotransposon; FIG. 4A; Tables 11-13). We chose these elements because they are all annotated as non-essential and no synthetic lethal interactions have been reported among them. This design required cloning 11 segments, which ranged in size from 3.8 kb to 21 kb (Tables 14 and 15), and programming them with appropriate adapters for assembly. As a reminder, we also exclude subtelomeres from synthetic chromosomes, which in BY comprise 61.7 kb and 24 protein-coding genes, 1 pseudogene, and 2 LTRs (Table 16). In total, the multiplex deletion design reduced ChrI by 39.9% (91.7 kb) and eliminated 45 genetic elements.

After cloning, we co-transformed the 11 segments into BY along with a centromere cassette and a linearized BAC/YAC vector. ADE1, a gene encoding an adenine biosynthesis enzyme whose loss of function causes colonies to accumulate red pigment, was present in one of the regions targeted for deletion. Thus, after native ChrI elimination, we picked 10 red colonies and checked each at all assembly junctions by PCR. While none of the red colonies possessed all deletions, a single red colony had nine of the 10 deletions, a finding confirmed by Oxford Nanopore Technologies sequencing (FIG. 4B). This strain with the multiplex deletion exhibited significantly slower growth than a strain with a synthetic BY ChrI lacking the deletions (212 and 94 minute doubling times, respectively; t-test, p-value=6.1×10⁻¹¹; FIG. 4C).

Of the 10 core chromosome regions targeted for deletion, the only region retained in the colony with nine deletions was between segments 6 and 7. The single gene residing in this region is SYN8, which encodes a SNARE protein involved in vesicle fusion with membranes³⁶. Re-examination of the red colonies found that SYN8 was retained in all 10, suggesting that it genetically interacts with one or more of the other deleted elements. To test for such an interaction, we deleted SYN8 from the strain with the other deletions. In this context, deleting SYN8 increased doubling time to 394 minutes, an 86% increase over the multiple deletion strain and a 319% increase over the strain with a synthetic BY ChrI lacking the deletions (FIG. 4C; Table 17). By contrast, SYN8 deletion had no effect on BY (difference=1 minute; t-test, p-value=0.96; FIG. 4C).

These results imply that multiplex deletion uncovered an unknown genetic interaction between SYN8 and other elements on ChrI, which converted the normally dispensable SYN8 into an quasi-essential gene⁷. Such unknown genetic interactions represent a major obstacle for efforts to streamline chromosomes and genomes, as they will cause strains carrying synthetic chromosomes to show slow growth and poor tractability in the lab. However, the SYN8 example shows that synthetic chromosomes generated by CReATiNG can overcome such unknown interactions via recombination with native chromosomes prior to native chromosome elimination (FIG. 4D).

CReATiNG makes it possible to build synthetic chromosomes with diverse designs using natural components. Because CReATiNG employs cloned segments of natural chromosomes as opposed to small DNA fragments synthesized de novo, it is substantially cheaper and faster than de novo chromosome synthesis. For example, some of the final chromosomes completed for this disclosure went from in silico design to in vivo testing within a month and cost less than five hundred dollars to produce. Although some synthetic chromosome designs will require complete chromosome reprogramming, which is not possible with CReATiNG, many will not. Indeed, we have shown here that CReATiNG can be used to study a variety of fundamental questions in genetics, genomics, and evolution. Moreover, we unexpectedly found an additional benefit of CReATiNG, which is that it can allow cells to recover from unknown design flaws via recombination between a synthetic chromosome and its native counterpart.

Most of the work in this disclosure involved simple synthetic chromosome designs in which only three segments were assembled. However, we also demonstrated in the multiplex deletion experiment that CReATiNG can be used to build synthetic chromosomes with complex designs involving 10 segments or more. Using CReATiNG to make synthetic chromosomes with complex designs could lead to important biological discoveries. For example, while here we synthetically recombined chromosomes at only two sites, this number could be increased, potentially by a large amount, facilitating fine scale genetic mapping of heritable traits within and between species. Similarly, more complex modifications of chromosome structure could be used to identify natural design principles governing chromosome architecture. CReATiNG can also likely be used to delete larger numbers of linked, non-adjacent regions from chromosomes than explored here, which could facilitate the streamlining of the yeast genome.

CReATiNG has potential applications beyond those shown in this disclosure. CReATiNG can be paired with de novo chromosome synthesis to enable projects that might not otherwise be feasible. For example, chromosomes with Saccharomyces architecture but sequences from other non-Saccharomyces species could be synthesized de novo and then recombined with Saccharomyces chromosomes using CReATiNG. Such an experiment would facilitate study of the genetic basis of reproductive isolation and trait differences between phylogenetically distant organisms. In addition, CReATiNG and de novo chromosome synthesis could be employed in combination to efficiently relocate genes in the same pathways, complexes, or cellular processes to common genetic modules. Further yet, with some modifications, CReATiNG in yeast may be employed to produce synthetic chromosomes, large DNA constructs, or gene variant libraries that could then be transferred to other systems, such as mammalian cells. These diverse applications highlight how CReATiNG democratizes the use of chromosome synthesis in biological research.

TABLE 1
Sequences of vectors pASC1, pASC2, and the centromere cassette.

| Vector | Sequence |
|---|---|
| Capture | ggccgtcgaccaattctcatgtttgacagcttatcatcgaatttctgccattcatccgcttattatcacttattcaggc |
| Vector | gtagcaaccaggcgtttaagggcaccaataactgccttaaaaaaattacgccccgccctgccactcatcgcag |
| (pASC1) | tactgttgtaattcattaagcattctgccgacatggaagccatcacaaacggcatgatgaacctgaatcgccagc |
| (SEQ ID | ggcatcagcaccttgtcgccttgcgtataatatttgcccatggtgaaaacgggggcgaagaagttgtccatattg |
| NO: 1) | gccacgtttaaatcaaaactg\gtgaaactcacccagggattggctgagacgaaaaacatattctcaataaacc |
| | ctttagggaaataggccaggttttcaccgtaacacgccacatcttgcgaatatatgtgtagaaactgccggaaat |
| | cgtcgtggtattcactccagagcgatgaaaacgtttcagtttgctcatggaaaacggtgtaacaagggtgaaca |
| | ctatcccatatcaccagctcaccgtctttcattgccatacgaaattccggatgagcattcatcaggcgggcaaga |
| | atgtgaataaaggccggataaaacttgtgcttatttttctttacggtctttaaaaaggccgtaatatccagctgaac |
| | ggtctggttataggtacattgagcaactgactgaaatgcctcaaaatgttctttacgatgccattgggatatatcaa |
| | cggtggtatatccagtgatttttttctccattttagcttccttagctcctgaaaatctcgataactcaaaaaatacgcc |
| | cggtagtgatcttatttcattatggtgaaagttggaacctcttacgtgccgatcaacgtctcattttcgccaaaagtt |
| | ggcccagggcttcccggtatcaacagggacaccaggatttatttattctgcgaagtgatcttccgtcacaggtat |
| | ttattcgcgataagctcatggagcggcgtaaccgtcgcacaggaaggacagagaaagcgcggatctgggaa |
| | gtgacggacagaacggtcaggacctggattggggaggcggttgccgccgctgctgctgacggtgtgacgtt |
| | ctctgttccggtcacaccacatacgttccgccattcctatgcgatgcacatgctgtatgccggtataccgctgaa |
| | agttctgcaaagcctgatgggacataagtccatcagttcaacggaagtctacacgaaggtttttgcgctggatgt |
| | ggctgcccggcaccgggtgcagtttgcgatgccggagtctgatgcggttgcgatgctgaaacaattatcctga |
| | gaataaatgccttggcctttatatggaaatgtggaactgagtggatatgctgtttttgtctgttaaacagagaagct |
| | ggctgttatccactgagaagcgaacgaaacagtcgggaaaatctcccattatcgtagagatccgcattattaatc |
| | tcaggagcctgtgtagcgtttataggaagtagtgttctgtcatgatgcctgcaagcggtaacgaaaacgatttga |
| | atatgccttcaggaacaatagaaatcttcgtgcggtgttacgttgaagtggagcggattatgtcagcaatggaca |
| | gaacaacctaatgaacacagaaccatgatgtggtctgtccttttacagccagtagtgctcgccgcagtcgagcg |
| | acagggcgaagccctcggctggttgccctcgccgctgggctggcggccgtctatggccctgcaaacgcgcc |
| | agaaacgccgtcgaagccgtgtgcgagacaccgcggccggccgccggcgttgtggatacctcgcggaaaa |
| | cttggccctcactgacagatgaggggcggacgttgacacttgaggggccgactcacccggcgcggcgttga |
| | cagatgaggggcaggctcgatttcggccggcgacgtggagctggccagcctcgcaaatcggcgaaaacgc |
| | ctgattttacgcgagtttcccacagatgatgtggacaagcctggggataagtgccctgcggtattgacacttgag |
| | gggcgcgactactgacagatgaggggcgcgatccttgacacttgaggggcagagtgctgacagatgaggg |
| | gcgcacctattgacatttgaggggctgtccacaggcagaaaatccagcatttgcaagggtttccgcccgtttttc |
| | ggccaccgctaacctgtcttttaacctgcttttaaaccaatatttataaaccttgtttttaaccagggctgcgccctg |
| | tgcgcgtgaccgcgcacgccgaaggggggtgcccccccttctcgaaccctcccggtcgagtgagcgagga |
| | agcaccagggaacagcacttatatattctgcttacacacgatgcctgaaaaaacttcccttggggttatccactta |
| | tccacggggatatttttataattattttttttatagtttttagatcttcttttttagagcgccttgtaggcctttatcc |
| | atgctggttctagagaaggtgttgtgacaaattgccctttcagtgtgacaaatcaccctcaaatgacagtcctgtctgt |
| | gacaaattgcccttaaccctgtgacaaattgccctcagaagaagctgttttttcacaaagttatccctgcttattgact |
| | cttttttatttagtgtgacaatctaaaaacttgtcacacttcacatggatctgtcatggcggaaacagcggttatcaat |
| | cacaagaaacgtaaaaatagcccgcgaatcgtccagtcaaacgacctcactgaggcggcatatagtctctccc |
| | gggatcaaaaacgtatgctgtatctgttcgttgaccagatcagaaaatctgatggcaccctacaggaacatgac |
| | ggtatctgcgagatccatgttgctaaatatgctgaaatattcggattgacctctgcggaagccagtaaggatata |
| | cggcaggcattgaagagtttcgcggggaaggaagtggttttttatcgccctgaagaggatgccggcgatgaa |
| | aaaggctatgaatcttttccttggtttatcaaacgtgcgcacagtccatccagagggctttacagtgtacatatcaa |
| | cccatatctcattcccttctttatcgggttacagaaccggtttacgcagtttcggcttagtgaaacaaaagaaatca |
| | ccaatccgtatgccatgcgtttatacgaatccctgtgtcagtatcgtaagccggatggctcaggcatcgtctctct |
| | gaaaatcgactggatcatagagcgttaccagctgcctcaaagttaccagcgtatgcctgacttccgccgccgct |
| | tcctgcaggtctgtgttaatgagatcaacagcagaactccaatgcgcctctcatacattgagaaaaagaaaggc |
| | cgccagacgactcatatcgtattttccttccgcgatatcacttccatgacgacaggatagtctgagggttatctgt |
| | cacagatttgagggtggttcgtcacatttgttctgacctactgagggtaatttgtcacagttttgctgtttccttcagc |
| | ctgcatggattttctcatactttttgaactgtaatttttaaggaagccaaatttgagggcagtttgtcacagttgattt |
| | ccttctctttcccttcgtcatgtgacctgatatcgggggttagttcgtcatcattgatgagggttgattatcacagttt |
| | attactctgaattggctatccgcgtgtgtacctctacctggagtttttcccacggtggatatttcttcttgcgctgagc |
| | gtaagagctatctgacagaacagttcttctttgcttcctcgccagttcgctcgctatgctcggttacacggctgcgg |
| | cgagcgctagtgataataagtgactgaggtatgtgctcttcttatctccttttgtagtgttgctcttattttaaacaac |
| | tttgcggttttttgatgactttgcgattttgttgttgctttgcagtaaattgcaagatttaataaaaaaacgcaaagca |
| | atgattaaaggatgttcagaatgaaactcatggaaacacttaaccagtgcataaacgctggtcatgaaatgacga |
| | aggctatcgccattgcacagtttaatgatgacagcccggaagcgaggaaaataacccggcgctggagaatag |
| | gtgaagcagcggatttagttggggtttcttctcaggctatcagagatgccgagaaagcagggcgactaccgca |
| | cccggatatggaaattcgaggacgggttgagcaacgtgttggttatacaattgaacaaattaatcatatgcgtga |
| | tgtgtttggtacgcgattgcgacgtgctgaagacgtatttccaccggtgatcggggttgctgcccataaaggtg |
| | gcgtttacaaaacctcagtttctgttcatcttgctcaggatctggctctgaaggggctacgtgttttgctcgtggaa |
| | ggtaacgacccccagggaacagcctcaatgtatcacggatgggtaccagatcttcatattcatgcagaagaca |
| | ctctcctgcctttctatcttggggaaaaggacgatgtcacttatgcaataaagcccacttgctggccggggcttg |
| | acattattccttcctgtctggctctgcaccgtattgaaactgagttaatgggcaaatttgatgaaggtaaactgccc |
| | accgatccacacctgatgctccgactggccattgaaactgttgctcatgactatgatgtcatagttattgacagcg |
| | cgcctaacctgggtatcggcacgattaatgtcgtatgtgctgctgatgtgctgattgttcccacgcctgctgagtt |
| | gtttgactacacctccgcactgcagtttttcgatatgcttcgtgatctgctcaagaacgttgatcttaaagggttcg |
| | agcctgatgtacgtattttgcttaccaaatacagcaatagtaatggctctcagtccccgtggatggaggagcaaa |
| | ttcgggatgcctggggaagcatggttctaaaaaatgttgtacgtgaaacggatgaagttggtaaaggtcagatc |
| | cggatgagaactgtttttgaacaggccattgatcaacgctcttcaactggtgcctggagaaatgctctttctatttg |
| | ggaacctgtctgcaatgaaattttcgatcgtctgattaaaccacgctgggagattagataatgaagcgtgcgcct |
| | gttattccaaaacatacgctcaatactcaaccggttgaagatacttcgttatcgacaccagctgccccgatggtg |
| | gattcgttaattgcgcgcgtaggagtaatggctcgcggtaatgccattactttgcctgtatgtggtcgggatgtga |
| | agtttactcttgaagtgctccggggtgatagtgttgagaagacctctcgggtatggtcaggtaatgaacgtgacc |
| | aggagctgcttactgaggacgcactggatgatctcatcccttcttttctactgactggtcaacagacaccggcgt |
| | tcggtcgaagagtatctggtgtcatagaaattgccgatgggagtcgccgtcgtaaagctgctgcacttaccgaa |
| | agtgattatcgtgttctggttggcgagctggatgatgagcagatggctgcattatccagattgggtaacgattatc |
| | gcccaacaagtgcttatgaacgtggtcagcgttatgcaagccgattgcagaatgaatttgctggaaatatttctg |
| | cgctggctgatgcggaaaatatttcacgtaagattattacccgctgtatcaacaccgccaaattgcctaaatcagt |
| | tgttgctcttttttctcaccccggtgaactatctgcccggtcaggtgatgcacttcaaaaagcctttacagataaag |
| | aggaattacttaagcagcaggcatctaaccttcatgagcagaaaaaagctggggtgatatttgaagctgaaga |
| | agttatcactcttttaacttctgtgcttaaaacgtcatctgcatcaagaactagtttaagctcacgacatcagtttgct |
| | cctggagcgacagtattgtataagggcgataaaatggtgcttaacctggacaggtctcgtgttccaactgagtgt |
| | atagagaaaattgaggccattcttaaggaacttgaaaagccagcaccctgatgcgaccacgttttagtctacgtt |
| | tatctgtctttacttaatgtcctttgttacaggccagaaagcataactggcctgaatattctctctgggcccactgttc |
| | cacttgtatcgtcggtctgataatcagactgggaccacggtcccactcgtatcgtcggtctgattattagtctggg |
| | accacggtcccactcgtatcgtcggtctgattattagtctgggaccacggtcccactcgtatcgtcggtctgata |
| | atcagactgggaccacggtcccactcgtatcgtcggtctgattattagtctgggaccatggtcccactcgtatcg |
| | tcggtctgattattagtctgggaccacggtcccactcgtatcgtcggtctgattattagtctggaaccacggtccc |
| | actcgtatcgtcggtctgattattagtctgggaccacggtcccactcgtatcgtcggtctgattattagtctgggac |
| | cacgatcccactcgtgttgtcggtctgattatcggtctgggaccacggtcccacttgtattgtcgatcagactatc |
| | agcgtgagactacgattccatcaatgcctgtcaagggcaagtattgacatgtcgtcgtaacctgtagaacggag |
| | taacctcggtgtgcggttgtatgcctgctgtggattgctgctgtgtcctgcttatccacaacattttgcgcacggtt |
| | atgtggaagcaaaaacaggaaggcaaaatgccgcaaaaaagggaataagggcgacacggaaatgttgaat |
| | actcatactcttcctttttcaatattattgaagcatttatcagggttattgtctcatgagcggatacatatttgaatgt |
| | atttagaaaaataaacaaataggggttccgcgcacatttccccgaaaagtgccacctgggtccttttcatcacgtgc |
| | tataaaaataattataatttaaattttttaatataaatatataaattaaaaatagaaagtaaaaaaagaaattaaagaa |
| | aaaatagtttttgttttccgaagatgtaaaagactctagggggatcgccaacaaatactaccttttatcttgctcttc |
| | ctgctctcaggtattaatgccgaattgtttcatcttgtctgtgtagaagaccacacacgaaaatcctgtgattttaca |
| | ttttacttatcgttaatcgaatgtatatctatttaatctgcttttcttgtctaataaatatatatgtaaagtacgcttt |
| | ttgttgaaattttttaaacctttgtttatttttttttcttcattccgtaactcttctaccttctttatttactttctaa |
| | aatccaaatacaaaacataaaaataaataaacacagagtaaattcccaaattattccatcattaaaagatacgaggcgc |
| | gtgtaagttacaggcaagcgatccgtcctaagaaaccattattatcatgacattaacctataaaaataggcgtatcacg |
| | aggccctttcgtctcgcgcgtttcggtgatgacggtgaaaacctctgacacatgcagctcccggagacggtcacagct |
| | tgtctgtaagcggatgccgggagcagacaagcccgtcagggcgcgtcagcgggtgttggcgggtgtcggg |
| | gctggcttaactatgcggcatcagagcagattgtactgagagtgcaccacgcttttcaattcaattcatcattttttt |
| | tttattcttttttttgatttcggtttctttgaaatttttttgattcggtaatctccgaacagaaggaagaacgaaggaa |
| | ggagcacagacttagattggtatatatacgcatatgtagtgttgaagaaacatgaaattgcccagtattcttaaccc |
| | aactgcacagaacaaaaacctgcaggaaacgaagataaatcatgtcgaaagctacatataaggaacgtgctg |
| | ctactcatcctagtcctgttgctgccaagctatttaatatcatgcacgaaaagcaaacaaacttgtgtgcttcattg |
| | gatgttcgtaccaccaaggaattactggagttagttgaagcattaggtcccaaaatttgtttactaaaaacacatgt |
| | ggatatcttgactgatttttccatggagggcacagttaagccgctaaaggcattatccgccaagtacaattttttac |
| | tcttcgaagacagaaaatttgctgacattggtaatacagtcaaattgcagtactctgcgggtgtatacagaatagc |
| | agaatgggcagacattacgaatgcacacggtgtggtgggcccaggtattgttagcggtttgaagcaggcggc |
| | agaagaagtaacaaaggaacctagaggccttttgatgttagcagaattgtcatgcaagggctccctatctactg |
| | gagaatatactaagggtactgttgacattgcgaagagcgacaaagattttgttatcggctttattgctcaaagaga |
| | catgggtggaagagatgaaggttacgattggttgattatgacacccggtgtgggtttagatgacaagggagac |
| | gcattgggtcaacagtatagaaccgtggatgatgtggtctctacaggatctgacattattattgttggaagagga |
| | ctatttgcaaagggaagggatgctaaggtagagggtgaacgttacagaaaagcaggctgggaagcatatttg |
| | agaagatgcggccagcaaaactaaaaaactgtattataagtaaatgcatgtatactaaactcacaaattagagct |
| | tcaatttaattatatcagttattaccctatgcggtgtgaaataccgcacagatgcgtaaggtagggataacagggt |
| | aattatagggcgaattcgagctcggtacccggctcgagtctagagtcgacctgcaggcatgcaattaccctgtt |
| | atccctactcactcaaaggcggtaatacggtt |
| Assembly | ttttgttgttgctttgcagtaaattgcaagatttaataaaaaaacgcaaagcaatgattaaaggatgttcagaatga |
| vector | aactcatggaaacacttaaccagtgcataaacgctggtcatgaaatgacgaaggctatcgccattgcacagttt |
| (pASC2) | aatgatgacagcccggaagcgaggaaaataacccggcgctggagaataggtgaagcagcggatttagttgg |
| (SEQ ID | ggtttcttctcaggctatcagagatgccgagaaagcagggcgactaccgcacccggatatggaaattcgagg |
| NO: 2) | acgggttgagcaacgtgttggttatacaattgaacaaattaatcatatgcgtgatgtgtttggtacgcgattgcga |
| | cgtgctgaagacgtatttccaccggtgatcggggttgctgcccataaaggtggcgtttacaaaacctcagtttct |
| | gttcatcttgctcaggatctggctctgaaggggctacgtgttttgctcgtggaaggtaacgacccccagggaac |
| | agcctcaatgtatcacggatgggtaccagatcttcatattcatgcagaagacactctcctgcctttctatcttggg |
| | gaaaaggacgatgtcacttatgcaataaagcccacttgctggccggggcttgacattattccttcctgtctggct |
| | ctgcaccgtattgaaactgagttaatgggcaaatttgatgaaggtaaactgcccaccgatccacacctgatgctc |
| | cgactggccattgaaactgttgctcatgactatgatgtcatagttattgacagcgcgcctaacctgggtatcggc |
| | acgattaatgtcgtatgtgctgctgatgtgctgattgttcccacgcctgctgagttgtttgactacacctccgcact |
| | gcagtttttcgatatgcttcgtgatctgctcaagaacgttgatcttaaagggttcgagcctgatgtacgtattttgctt |
| | accaaatacagcaatagtaatggctctcagtccccgtggatggaggagcaaattcgggatgcctggggaagc |
| | atggttctaaaaaatgttgtacgtgaaacggatgaagttggtaaaggtcagatccggatgagaactgtttttgaa |
| | caggccattgatcaacgctcttcaactggtgcctggagaaatgctctttctatttgggaacctgtctgcaatgaaa |
| | ttttcgatcgtctgattaaaccacgctgggagattagataatgaagcgtgcgcctgttattccaaaacatacgctc |
| | aatactcaaccggttgaagatacttcgttatcgacaccagctgccccgatggtggattcgttaattgcgcgcgta |
| | ggagtaatggctcgcggtaatgccattactttgcctgtatgtggtcgggatgtgaagtttactcttgaagtgctcc |
| | ggggtgatagtgttgagaagacctctcgggtatggtcaggtaatgaacgtgaccaggagctgcttactgagga |
| | cgcactggatgatctcatcccttcttttctactgactggtcaacagacaccggcgttcggtcgaagagtatctggt |
| | gtcatagaaattgccgatgggagtcgccgtcgtaaagctgctgcacttaccgaaagtgattatcgtgttctggtt |
| | ggcgagctggatgatgagcagatggctgcattatccagattgggtaacgattatcgcccaacaagtgcttatga |
| | acgtggtcagcgttatgcaagccgattgcagaatgaatttgctggaaatatttctgcgctggctgatgcggaaaa |
| | tatttcacgtaagattattacccgctgtatcaacaccgccaaattgcctaaatcagttgttgctcttttttctcacccc |
| | ggtgaactatctgcccggtcaggtgatgcacttcaaaaagcctttacagataaagaggaattacttaagcagca |
| | ggcatctaaccttcatgagcagaaaaaagctggggtgatatttgaagctgaagaagttatcactcttttaacttct |
| | gtgcttaaaacgtcatctgcatcaagaactagtttaagctcacgacatcagtttgctcctggagcgacagtattgt |
| | ataagggcgataaaatggtgcttaacctggacaggtctcgtgttccaactgagtgtatagagaaaattgaggcc |
| | attcttaaggaacttgaaaagccagcaccctgatgcgaccacgttttagtctacgtttatctgtctttacttaatgtc |
| | ctttgttacaggccagaaagcataactggcctgaatattctctctgggcccactgttccagcaaaaacaggaag |
| | gcaaaatgccgcaaaaaagggaataagggcgacacggaaatgttgaatactcatactcttcctttttcaatattat |
| | tgaagcatttatcagggttattgtctcatgagcggatacatatttgaatgtatttagaaaaataaacaaataggggt |
| | tccgcgcacatttccccgaaaagtgccacctgggtccttttcatcacgtgctataaaaataattataatttaaattttt |
| | taatataaatatataaattaaaaatagaaagtaaaaaaagaaattaaagaaaaaatagtttttgttttccgaagatgt |
| | aaaagactctagggggatcgccaacaaatactaccttttatcttgctcttcctgctctcaggtattaatgccgaatt |
| | gtttcatcttgtctgtgtagaagaccacacacgaaaatcctgtgattttacattttacttatcgttaatcgaatgtata |
| | tctatttaatctgcttttcttgtctaataaatatatatgtaaagtacgctttttgttgaaattttttaaacctttgttt |
| | atttttttttcttcattccgtaactcttctaccttctttatttactttctaaaatccaaatacaaaacataaaaataaa |
| | taaacacagagtaaattcccaaattattccatcattaaaagatacgaggcgcgtgtaagttacaggcaagcgatccgtc |
| | ctaagaaaccattattatcatgacattaacctataaaaataggcgtatcacgaggccctttcgtctcgcgcgtttcggt |
| | gatgacggtggtatcgtttgaacacggcattagtcagggaagtcataacacagtcctttcccgcaattttctttttcta |
| | ttactcttggcctcctctagtacactctatatttttttatgcctcggtaatgattttcatttttttttttcccctagcg |
| | gatgactctttttttttcttagcgattggcattatcacataatgaattatacattatataaagtaatgtgatttcttcg |
| | aagaatatactaaaaaatgagcaggcaagataaacgaaggcaaagatgacagagcagaaagccctagtaaagcgtatta |
| | caaatgaaaccaagattcagattgcgatctctttaaagggtggtcccctagcgatagagcactcgatcttcccagaa |
| | aaagaggcagaagcagtagcagaacaggccacacaatcgcaagtgattaacgtccacacaggtatagggttt |
| | ctggaccatatgatacatgctctggccaagcattccggctggtcgctaatcgttgagtgcattggtgacttacac |
| | atagacgaccatcacaccactgaagactgcgggattgctctcggtcaagcttttaaagaggccctactggcgc |
| | gtggagtaaaaaggtttggatcaggatttgcgcctttggatgaggcactttccagagcggtggtagatctttcga |
| | acaggccgtacgcagttgtcgaacttggtttgcaaagggagaaagtaggagatctctcttgcgagatgatccc |
| | gcattttcttgaaagctttgcagaggctagcagaattaccctccacgttgattgtctgcgaggcaagaatgatcat |
| | caccgtagtgagagtgcgttcaaggctcttgcggttgccataagagaagccacctcgcccaatggtaccaacg |
| | atgttccctccaccaaaggtgttcttatgtagtgacaccgattatttaaagctgcagcatacgatatatatacatgt |
| | gtatatatgtatacctatgaatgtcagtaagtatgtatacgaacagtatgatactgaagatgacaaggtaatgcat |
| | cattctatacgtgtcattctgaacgaggcgcgctttcctttaccgcatcaggaaattgtaagtgtgaaataccgca |
| | cagatgcgtaaggtagggataacagggtaattatagggcgaattctattttacgcgcaagccggacccgcgct |
| | tgcacggctgaggtgcgtagggaacgtcgcaaagaggcatgaatattcagcagcacagaacaaattaatcac |
| | ttaattaaatcgtcgatggatccacttgatacctagttaacccacgggtgtggaaaagctccctaagggaggaa |
| | cggacgccggaaggttttcctgagtggcactcacacattatggtgtcaggcatgcattaccctgttatccctact |
| | cactcaaaggcggtaatacggttggccgtcgaccaattctcatgtttgacagcttatcatcgaatttctgccattca |
| | tccgcttattatcacttattcaggcgtagcaaccaggcgtttaagggcaccaataactgccttaaaaaaattacgc |
| | cccgccctgccactcatcgcagtactgttgtaattcattaagcattctgccgacatggaagccatcacaaacgg |
| | catgatgaacctgaatcgccagcggcatcagcaccttgtcgccttgcgtataatatttgcccatggtgaaaacg |
| | ggggcgaagaagttgtccatattggccacgtttaaatcaaaactggtgaaactcacccagggattggctgaga |
| | cgaaaaacatattctcaataaaccctttagggaaataggccaggttttcaccgtaacacgccacatcttgcgaat |
| | atatgtgtagaaactgccggaaatcgtcgtggtattcactccagagcgatgaaaacgtttcagtttgctcatgga |
| | aaacggtgtaacaagggtgaacactatcccatatcaccagctcaccgtctttcattgccatacgaaattccggat |
| | gagcattcatcaggcgggcaagaatgtgaataaaggccggataaaacttgtgcttatttttctttacggtctttaaa |
| | aaggccgtaatatccagctgaacggtctggttataggtacattgagcaactgactgaaatgcctcaaaatgttctt |
| | tacgatgccattgggatatatcaacggtggtatatccagtgatttttttctccattttagcttccttagctcctgaaaa |
| | tctcgataactcaaaaaatacgcccggtagtgatcttatttcattatggtgaaagttggaacctcttacgtgccgatc |
| | aacgtctcattttcgccaaaagttggcccagggcttcccggtatcaacagggacaccaggatttatttattctgcg |
| | aagtgatcttccgtcacaggtatttattcgcgataagctcatggagcggcgtaaccgtcgcacaggaaggaca |
| | gagaaagcgcggatctgggaagtgacggacagaacggtcaggacctggattggggaggcggttgccgcc |
| | gctgctgctgacggtgtgacgttctctgttccggtcacaccacatacgttccgccattcctatgcgatgcacatg |
| | ctgtatgccggtataccgctgaaagttctgcaaagcctgatgggacataagtccatcagttcaacggaagtcta |
| | cacgaaggtttttgcgctggatgtggctgcccggcaccgggtgcagtttgcgatgccggagtctgatgcggtt |
| | gcgatgctgaaacaattatcctgagaataaatgccttggcctttatatggaaatgtggaactgagtggatatgctg |
| | tttttgtctgttaaacagagaagctggctgttatccactgagaagcgaacgaaacagtcgggaaaatctcccatt |
| | atcgtagagatccgcattattaatctcaggagcctgtgtagcgtttataggaagtagtgttctgtcatgatgcctgc |
| | aagcggtaacgaaaacgatttgaatatgccttcaggaacaatagaaatcttcgtgcggtgttacgttgaagtgg |
| | agcggattatgtcagcaatggacagaacaacctaatgaacacagaaccatgatgtggtctgtccttttacagcc |
| | agtagtgctcgccgcagtcgagcgacagggcgaagccctcggctggttgccctcgccgctgggctggcgg |
| | ccgtctatggccctgcaaacgcgccagaaacgccgtcgaagccgtgtgcgagacaccgcggccggccgcc |
| | ggcgttgtggatacctcgcggaaaacttggccctcactgacagatgaggggcggacgttgacacttgagggg |
| | ccgactcacccggcgcggcgttgacagatgaggggcaggctcgatttcggccggcgacgtggagctggcc |
| | agcctcgcaaatcggcgaaaacgcctgattttacgcgagtttcccacagatgatgtggacaagcctggggata |
| | agtgccctgcggtattgacacttgaggggcgcgactactgacagatgaggggcgcgatccttgacacttgag |
| | gggcagagtgctgacagatgaggggcgcacctattgacatttgaggggctgtccacaggcagaaaatccag |
| | catttgcaagggtttccgcccgtttttcggccaccgctaacctgtcttttaacctgcttttaaaccaatatttataaac |
| | cttgtttttaaccagggctgcgccctgtgcgcgtgaccgcgcacgccgaaggggggtgcccccccttctcgaa |
| | ccccccggtcgagtgagcgaggaagcaccagggaacagcacttatatattctgcttacacacgatgcctgaa |
| | aaaacttcccttggggttatccacttatccacggggatatttttataattattttttttatagtttttagatcttcttt |
| | tttagagcgccttgtaggcctttatccatgctggttctagagaaggtgttgtgacaaattgccctttcagtgtgacaaa |
| | tcaccctcaaatgacagtcctgtctgtgacaaattgcccttaaccctgtgacaaattgccctcagaagaagctgtttt |
| | ttcacaaagttatccctgcttattgactcttttttatttagtgtgacaatctaaaaacttgtcacacttcacatggatc |
| | tgtcatggcggaaacagcggttatcaatcacaagaaacgtaaaaatagcccgcgaatcgtccagtcaaacgacc |
| | tcactgaggcggcatatagtctctcccgggatcaaaaacgtatgctgtatctgttcgttgaccagatcagaaaat |
| | ctgatggcaccctacaggaacatgacggtatctgcgagatccatgttgctaaatatgctgaaatattcggattga |
| | cctctgcggaagccagtaaggatatacggcaggcattgaagagtttcgcggggaaggaagtggttttttatcg |
| | ccctgaagaggatgccggcgatgaaaaaggctatgaatcttttccttggtttatcaaacgtgcgcacagtccatc |
| | cagagggctttacagtgtacatatcaacccatatctcattcccttctttatcgggttacagaaccggtttacgcagt |
| | ttcggcttagtgaaacaaaagaaatcaccaatccgtatgccatgcgtttatacgaatccctgtgtcagtatcgtaa |
| | gccggatggctcaggcatcgtctctctgaaaatcgactggatcatagagcgttaccagctgcctcaaagttacc |
| | agcgtatgcctgacttccgccgccgcttcctgcaggtctgtgttaatgagatcaacagcagaactccaatgcgc |
| | ctctcatacattgagaaaaagaaaggccgccagacgactcatatcgtattttccttccgcgatatcacttccatga |
| | cgacaggatagtctgagggttatctgtcacagatttgagggtggttcgtcacatttgttctgacctactgagggta |
| | atttgtcacagttttgctgtttccttcagcctgcatggattttctcatactttttgaactgtaatttttaaggaagcca |
| | aatttgagggcagtttgtcacagttgatttccttctctttcccttcgtcatgtgacctgatatcgggggttagttcgtc |
| | atcattgatgagggttgattatcacagtttattactctgaattggctatccgcgtgtgtacctctacctggagtttttc |
| | ccacggtggatatttcttcttgcgctgagcgtaagagctatctgacagaacagttcttctttgcttcctcgccagttcg |
| | ctcgctatgctcggttacacggctgcggcgagcgctagtgataataagtgactgaggtatgtgctcttcttatctc |
| | cttttgtagtgttgctcttattttaaacaactttgcggttttttgatgactttgcga |
| Centromere | tctaaactagaaagttaccgttagtcttactgatccgaaccgtcattagttactagaggaaacgtgtcaagaatgc |
| Cassette | cagccgatcccacattcttaattactcagtggaacgaaaactcacgttaagggattttggtcatgagattatcaaa |
| (SEQ ID | aaggatcttcacctagatccttttaaattaaaaatgaagttttaaatcaatctaaagtatatatgagtaaacttggtct |
| NO: 3) | gacagttaccaatgcttaatcagtgaggcacctatctcagcgatctgtctatttcgttcatccatagttgcctgact |
| | gcccgtcgtgtagataactacgatacgggagggcttaccatctggccccagtgctgcaatgataccgcgaga |
| | cccacgctcaccggctccagatttatcagcaataaaccagccagccggaagggccgagcgcagaagtggtc |
| | ctgcaactttatccgcctccatccagtctattaattgttgccgggaagctagagtaagtagttcgccagttaatagt |
| | ttgcgcaacgttgttgccattgctacaggcatcgtggtgtcacgctcgtcgtttggtatggcttcattcagctccg |
| | gttcccaacgatcaaggcgagttacatgatcccccatgttgtgaaaaaaagcggttagctccttcggtcctccg |
| | atcgttgtcagaagtaagttggccgcagtgttatcactcatggttatggcagcactgcataattctcttactgtcat |
| | gccatccgtaagatgcttttctgtgactggtgagtactcaaccaagtcattctgagaatagtgtatgcggcgacc |
| | gagttgctcttgcccggcgtcaatacgggataataccgcgccacatagcagaactttaaaagtgctcatcattg |
| | gaaaacgttcttcggggcgaaaactctcaaggatcttaccgctgttgagatccagttcgatgtaacccactcgtg |
| | cacccaactgatcttcagcatcttttactttcaccagcgtttctgggtgagcaaaaacaggaaggcaaaatgccg |
| | caaaaaagggaataagggcgacacggaaatgttgaatactcatactcttcctttttcaatattattgaagcatttat |
| | cagggttattgtctcatgagcggatacatatttgaatgtatttagaaaaataaacaaataggggttccgcgcacat |
| | ttccccgaaaagtgccacctgggtccttttcatcacgtgctataaaaataattataatttaaattttttaatataaata |
| | tataaattaaaaatagaaagtaaaaaaagaaattaaagaaaaaatagtttttgttttccgaagatgtaaaagactct |
| | agggggatcgccaacaaatactaccttttatcttgctcttcctgctctcaggtattaatgccgaattgtttcatcttgt |
| | ctgtgtagaagaccacacacgaaaatcctgtgattttacattttacttatcgttaatcgaatgtatatctatttaatct |
| | gcttttcttgtctaataaatatatatgtaaagtacgctttttgttgaaattttttaaacctttgtttatttttttttct |
| | tcattccgtaactcttctaccttctttatttactttctaaaatccaaatacaaaacataaaaataaataaacacagagt |
| | aaattcccaaattattccatcattaaaagatacgaggcgcgtgtaagttacaggcaagcgatccgtcctaagaaaccat |
| | gacgagtacggtgggtaggccggccagcgacatggaggcccagaataccctccttgacagtcttgacgtgcgcag |
| | ctcaggggcatgatgtgactgtcgcccgtacatttagcccatacatccccatgtataatcatttgcatccatacatt |
| | ttgatggccgcacggcgcgaagcaaaaattacggctcctcgctgcagacctgcgagcagggaaacgctccc |
| | ctcacagacgcgttgaattgtccccacgccgcgcccctgtagagaaatataaaaggttaggatttgccactgag |
| | gttcttctttcatatacttccttttaaaatcttgctaggatacagttctcacatcacatccgaacataaacaaccatgg |
| | gtaaggaaaagactcacgtttcgaggccgcgattaaattccaacatggatgctgatttatatgggtataaatggg |
| | ctcgcgataatgtcgggcaatcaggtgcgacaatctatcgattgtatgggaagcccgatgcgccagagttgttt |
| | ctgaaacatggcaaaggtagcgttgccaatgatgttacagatgagatggtcagactaaactggctgacggaatt |
| | tatgcctcttccgaccatcaagcattttatccgtactcctgatgatgcatggttactcaccactgcgatccccggc |
| | aaaacagcattccaggtattagaagaatatcctgattcaggtgaaaatattgttgatgcgctggcagtgttcctgc |
| | gccggttgcattcgattcctgtttgtaattgtccttttaacagcgatcgcgtatttcgtctcgctcaggcgcaatcac |
| | gaatgaataacggtttggttgatgcgagtgattttgatgacgagcgtaatggctggcctgttgaacaagtctgga |
| | aagaaatgcataagcttttgccattctcaccggattcagtcgtcactcatggtgatttctcacttgataaccttatttt |
| | tgacgaggggaaattaataggttgtattgatgttggacgagtcggaatcgcagaccgataccaggatcttgccat |
| | cctatggaactgcctcggtgagttttctccttcattacagaaacggctttttcaaaaatatggtattgataatcctgat |
| | atgaataaattgcagtttcatttgatgctcgatgagtttttctaatcagtactgacaataaaaagattcttgttttcaa |
| | gaacttgtcatttgtatagtttttttatattgtagttgttctattttaatcaaatgttagcgtgatttatatttttttt |
| | cgcctcgacatcatctgcccagatgcgaagttaagtgcgcagaaagtaatatcatgcgtcaatcgtatgtgaatgctgg |
| | tcgctatactgctgtcgattcgatactaacgccgccatccagtgtcgaaaactgcttggcaacctaatgctannanna |
| | nnannannannannannannannataccgttcgtataatgtgtactatacgaagttattcaatcggtgtcactt |
| | atggataacttcgtataatgtatgctatacgaacggtaacttgatacctagttaacccacgggtgtggaaaagctc |
| | cctaagggaggaacggacgccggaaggttttcctgagtggcactcacacattatggtgtcag |

TABLE 2
Cloning cassettes for segments 1 through 3 in all experiments except multiplex deletion.
Each cassette contains a pair of upstream and downstream adapters flanking segment-specific
homology arms separated by XhoI and AvrII sites used for vector linearization. The same cloning
cassettes were used in BY and RM, while different cloning cassettes were used for S. paradoxus.

| Cloning | |
|---|---|
| cassette | Sequence |
| BY or RM | tattttacgcgcaagccggacccgcgcttgcacggctgaggtgcgtagggaacgtcgcaaagaggcatga |
| segment 1 | atattcagcagcacagaacaaattaatcacgagccaatgagattttaaagggtatattacttatcttatcg |
| (SEQ ID NO: 4) | ataagcagtattgatattaaagggacagttttatcgttggttaatatggaaaaagtgatgaccatgatgc |
| | ctttcttaaaaagagtatttctttttatttcactttcacctcgagagtcctaggtatttttaggaaattgatgatac |
| | cgtgggaataatgagagataacataaataaagtagcagaaagaggtgaaagattaacgtccattgaagataaa |
| | gccgataacctagcggtctcagcccaaggctttaagaggggtgccaatagggtccttacatatcctctacacc |
| | gagcgcgtcgacccgtcgaatgaatgtgagttgcgtgttgttccttcggaaatgttgctaaaaaacagagcc |
| | attcctttt |
| BY or RM | cttacatatcctctacaccgagcgcgtcgacccgtcgaatgaatgtgagttgcgtgttgttccttcggaaatgtt |
| segment 2 | gctaaaaaacagagccattcctttttatatgtatatatacatatatacatatatgggcgtatatttactttgtt |
| (SEQ ID NO: 5) | cttattttctgtctaattttataattttactgacagtagctaagccctctgtattgctgttctgtgttattgcact |
| | agtgtcataacgcagatggtttttactcgagagtcctagctatttgtacgagttcgtcaggataaatcgtcagta |
| | ccatttttcttgtggctagttggcttcaaccaaacgtcctcttctctcttatggcaagaagaaagttatatgtgtgact |
| | ggttgtttatttcactttcgcgactgaaagcgccgtctaaactagaaagttaccgttagtcttactgatccgaacc |
| | gtcattagttactagaggaaacgtgtcaagaatgccagccgatcccacattcttaatta |
| BY or RM | aatcaaagggaagtcgaataaaggtcatccagggaattagccgttttcgaccaagtctttacgggagtaga |
| segment 3 | agacgaatcctctcgtgtgctggcccaggaaattaagatagttattcgagtgattgccaaatatcatgttct |
| (SEQ ID NO: 6) | acttcgaagacttatagctaattaattttttcataatgaaggtgtcgttaattgttctgattagtaacatgaa |
| | actcaaaaatcatcaaaaaaagaaaagctaaatgctcgagagtcctaggaaaggatcatggctggcagc |
| | gcccacaacaattaaactctgttactatcaaaaaacattgagcccaagaatggaataaaattttcactacacctcg |
| | gacatggatttgtacatgtcctattatcctgtaattttgacatatactgatatgacttgatacctagttaacccacgg |
| | gtgtggaaaagctccctaagggaggaacggacgccggaaggttttcctgagtggcactcacacattatggt |
| | gtcag |
| S. paradoxus | tattttacgcgcaagccggacccgcgcttgcacggctgaggtgcgtagggaacgtcgcaaagaggcatga |
| segment 1 | atattcagcagcacagaacaaattaatcactagaaacggctgacaaccattttgttagtcctcggaaaat |
| (SEQ ID NO: 7) | ctccgaacaaatgagaaagcaaaatataataacacagagtttggcgagaagaagatatatgataacta |
| | ctattaaggaccctcgagagtcctaggtagaaacggctgacaaccattttgttagtcctcggaaaatctccgaa |
| | caaatgagaaagcaaaatataataacacagagtttggcgagaagaagatatatgataactactattaaggaccc |
| | ttacatatcctctacaccgagcgcgtcgacccgtcgaatgaatgtgagttgcgtgttcttccttcggaaatgttg |
| | ctaaaaaacagagccattcctttt |
| S. paradoxus | cttacatatcctctacaccgagcgcgtcgacccgtcgaatgaatgtgagttgcgtgttgttccttcggaaatgtt |
| segment 2 | gctaaaaaacagagccattcctttttatatatgagagcgtatatgtactttgttgtttttattttttcctgcttag |
| (SEQ ID NO: 8) | ttctataatcttattgacatttgtcaagccctctgtattgcagttatgtggtattgcaccggtgtcatactcga |
| | gagtcctaggtcacatgttacttttataaaataattatattatattttaaatatatcaaagtttctttcattttttaga |
| | ttactttaagataataaatatatgtttttggtttccgaaacgtttaaaatatctaaactagaaagttaccgttagtctt |
| | actgatccgaaccgtcattagttactagaggaaacgtgtcaagaatgccagccgatcccacattcttaatta |
| S. paradoxus | aatcaaagggaagtcgaataaaggtcatccagggaattagccgttttcgaccaagtctttacgggagtaga |
| segment 3 | agacgaatcctctcgtgtgctggcccaggccagcgatagcccattgtgtaatgtgtatttctaaccatcaa |
| (SEQ ID NO: 9) | acactatacctctacgccaaatagacaacgcaagacactctataaacggaaacccaatgcaccataaa |
| | tcattaaggactcgagagtcctaggccagcgatagcccattgtgtaatgtgtatttctaaccatcaaacactata |
| | cctctacgccaaatagacaacgcaagacactctataaacggaaacccaatgcaccataaatcattaaggaactt |
| | gatacctagttaacccacgggtgtggaaaagctccctaagggaggaacggacgccggaaggttttcctga |
| | gtggcactcacacattatggtgtcag |

Upstream adapter (italicized); upstream homology arm (bold); downstream homology (underlined); and downstream adapter (italicized and underlined)

TABLE 3
gRNAs used to clone the three segments in the initial chromosome substitution,
synthetic recombination, and restructuring experiments. Column 1 (‘gRNAs’)
lists the gRNAs IDs. The annotations S1, S2 and S3 represent segment 1, 2
and 3, respectively. The ‘up’ and ‘down’ annotations refer to the side of a
segment targeted by a guide. Column 2 (‘sequence’) lists the target sequence
of each gRNA.

| gRNAs | Sequence |
|---|---|
| S. cerevisiae S1 gRNA1 up | cattaaaaagaaggcatagg (SEQ ID NO: 10) |
| S. cerevisiae S1 gRNA2 up | tttgaagaaaggaaaaaatg (SEQ ID NO: 11) |
| S. cerevisiae S1 gRNA3 up | ggccaatcaagcaaactgaa (SEQ ID NO: 12) |
| S. cerevisiae S1 gRNA1 down | tctaaaaatgaagatgtgtc (SEQ ID NO: 13) |
| S. cerevisiae S1 gRNA2 down | acacagaacagcaatacaga (SEQ ID NO: 14) |
| S. cerevisiae S1 gRNA3 down | aacacagaacagcaatacag (SEQ ID NO: 15) |
| S. cerevisiae S2 gRNA1 up | ttttagatccttgtaccaca (SEQ ID NO: 16) |
| S. cerevisiae S2 gRNA2 up | tctaaaaatgaagatgtgtc (SEQ ID NO: 17) |
| S. cerevisiae S2 gRNA1 down | ctgcattttcaaataccgct (SEQ ID NO: 18) |
| S. cerevisiae S2 gRNA2 down | gttttttgcatcataaaggg (SEQ ID NO: 19) |
| S. cerevisiae S2 gRNA3 down | tcaatataataagcatacta (SEQ ID NO: 20) |
| S. cerevisiae S3 gRNA1 up | cacctttcgtctgctgcctc (SEQ ID NO: 21) |
| S. cerevisiae S3 gRNA2 up | cacccgaggcagcagacgaa (SEQ ID NO: 22) |
| S. cerevisiae S3 gRNA3 up | ataatttgaaaaaacacccg (SEQ ID NO: 23) |
| S. cerevisiae S3 gRNA1 down | atcctttttttatttgaaag (SEQ ID NO: 24) |
| S. cerevisiae S3 gRNA2 down | aataaaaaaaggatcatggc (SEQ ID NO: 25) |
| S. cerevisiae S3 gRNA3 down | aaaaacattgagcccaagaa (SEQ ID NO: 26) |
| S. paradoxus S1 gRNA1 up | cattataaaaattaaaaccg (SEQ ID NO: 27) |
| S.paradoxus S1 gRNA2 up | gattaacggagctgcccaag (SEQ ID NO: 28) |
| S.paradoxus S1 gRNA3 up | atggaaacctccttctgtgg (SEQ ID NO: 29) |
| S.paradoxus S1 gRNA1 down | caatgtttaactaatactct (SEQ ID NO: 30) |
| S.paradoxus S1 gRNA2 down | tctctgctcaaggtttcaag (SEQ ID NO: 31) |
| S.paradoxus S1 gRNA3 down | atagggtgaggaaagccatg (SEQ ID NO: 32) |
| S.paradoxus S2 gRNA1 up | ttagtcgatagaaattcact (SEQ ID NO: 33) |
| S.paradoxus S2 gRNA2 up | gaatactatatatatatagg (SEQ ID NO: 34) |
| S.paradoxus S2 gRNA3 up | ataagattatagaactaagc (SEQ ID NO: 35) |
| S.paradoxus S2 gRNA1 down | atacccgtccagaatgtgag (SEQ ID NO: 36) |
| S.paradoxus S2 gRNA2 down | tacccgtccagaatgtgaga (SEQ ID NO: 37) |
| S.paradoxus S2 gRNA3 down | atagattttctgaccagtag (SEQ ID NO: 38) |
| S.paradoxus S3 gRNA1 up | cataactaactcgattcgca (SEQ ID NO: 39) |
| S.paradoxus S3 gRNA2 up | atggattgccaacgatgatg (SEQ ID NO: 40) |
| S.paradoxus S3 gRNA3 up | actgagagccaagaattcaa (SEQ ID NO: 41) |
| S.paradoxus S3 gRNA1 down | aaagttgagatattctggat (SEQ ID NO: 42) |
| S.paradoxus S3 gRNA2 down | caaacttcatgcaggatgct (SEQ ID NO: 43) |
| S.paradoxus S3 gRNA3 down | aatttcggaaaaaaagtccc (SEQ ID NO: 44) |

TABLE 4
Primers used to confirm cloning of segments 1 through 3 for all experiments
except multiplex deletion. Column 1 (‘Primers’) lists primers IDs. Column 2
(‘Sequence’) contains the nucleotide sequence of each primer.

| Primers | Sequence |
|---|---|
| S. cerevisiae S1 clone check REV | ggaagcgtaggccaccct (SEQ ID NO: 45) |
| S. cerevisiae S1 clone check FOR | tccgagcacgatgaagaacga (SEQ ID NO: 46) |
| S. cerevisiae S2 clone check REV | aagcctgctaacaagggcga (SEQ ID NO: 47) |
| S. cerevisiae S2 clone check FOR | ccagttacctgcgccaaatc (SEQ ID NO: 48) |
| S. cerevisiae S3 clone check REV | ctggcttcacccactgcaga (SEQ ID NO: 49) |
| S. cerevisiae S3 clone check FOR | ccatgagctgcgtggcatt (SEQ ID NO: 50) |
| S. paradoxus S1 clone check REV | ccgatgggtgaaagcgtagc (SEQ ID NO: 51) |
| S. paradoxus S1 clone check FOR | gaacggcctcagaatgtgcag (SEQ ID NO: 52) |
| S. paradoxus S2 clone check REV | gaggcgaagctggagtaccg (SEQ ID NO: 53) |
| S. paradoxus S2 clone check FOR | aagcgccccttctgatgca (SEQ ID NO: 54) |
| S. paradoxus S3 clone check REV | aacagtgacaccacaccctct (SEQ ID NO: 55) |
| S. paradoxus S3 clone check FOR | cttgttggcgtgtgaggagg (SEQ ID NO: 56) |
| Centromere check REV | aaagttgcaggaccacttctgc (SEQ ID NO: 57) |
| Centromere check FRW | cccagatgcgaagttaagtg (SEQ ID NO: 58) |
| pASC1 check FRW | caagggagacgcattgggtca (SEQ ID NO: 59) |
| pASC1 check REV | gcctggttgctacgcctga (SEQ ID NO: 60) |

TABLE 5
Cloning efficiency of chromosome I segments 1 through 3 from BY, RM,
and S. paradoxus. Column 2 (‘Size’) lists the size of each target
segment from all 3 different strains. Column 2 (‘Upstream junction
check’) lists the number of positive PCR reactions from the upstream
junction of a target segment and the capture vector. Column 3
(‘Downstream junction check’) lists the number of positive PCR
reactions from the downstream junction of a target segment and the
capture vector. Column 4 (‘Cloning efficiency’) lists the overall
efficiency of segment cloning based on the positive rate of PCR
reaction on both junctions up and downstream.

| Cloned | Size | Upstream | Downstream | Cloning |
|---|---|---|---|---|
| segment | (kb) | junction check | junction check | efficiency |
| S.par 1 | 56.6 | 5/5 (100%) | 5/5 (100%) | 100% |
| S.par 2 | 63.9 | 5/5 (100%) | 4/5 (80%) | 80% |
| S.par 3 | 53.9 | 5/5 (100%) | 5/5 (100%) | 100% |
| BY 1 | 56.5 | 5/5 (100%) | 5/5 (100%) | 100% |
| BY 2 | 63.4 | 4/5 (80%) | 4/5 (80%) | 80% |
| BY 3 | 45.1 | 5/5 (100%) | 5/5 (100%) | 100% |
| RM 1 | 56.5 | 5/5 (100%) | 5/5 (100%) | 100% |
| RM 2 | 63.4 | 5/5 (100%) | 5/5 (100%) | 100% |
| RM 3 | 39.6 | 5/5 (100%) | 5/5 (100%) | 100% |

TABLE 6
Efficiency of assembly of synthetic chimera ChrI
in BY4742 and elimination of its native ChrI.

| | | | | | | Native ChrI | |
|---|---|---|---|---|---|---|---|
| | Segment | Segment | Segment | Assembly | | elimination | |
| Strain | 1 | 2 | 3 | Efficiency | | efficiency | |
| IEY394 | S. par | S. par | S. par | 4/5 | (75%) | 4/4 | (100%) |
| IEY395 | S. par | RM | S. par | 3/3 | (100%) | 3/3 | (100%) |
| IEY396 | S. par | S. par | RM | 3/3 | (100%) | 2/3 | (66%) |
| IEY397 | S. par | RM | RM | 1/3 | (33%) | 1/1 | (100%) |
| IEY398 | RM | RM | RM | 2/3 | (66%) | 2/2 | (100%) |
| IEY399 | RM | S. par | RM | 2/3 | (66%) | 2/2 | (100%) |
| IEY400 | RM | RM | S. par | 1/3 | (33%) | 1/1 | (100%) |
| IEY401 | RM | S. par | S. par | 3/3 | (100%) | 2/3 | (66%) |
| IEY402 | BY | BY | BY | 2/3 | (66%) | 1/2 | (50%) |
| IEY403 | BY | BY | RM | 1/5 | (20%) | 1/1 | (100%) |
| IEY404 | BY | BY | S. par | 2/5 | (40%) | 1/2 | (50%) |
| IEY405 | BY | RM | BY | 2/3 | (66%) | 2/2 | (100%) |
| IEY406 | BY | RM | RM | 1/3 | (33%) | 1/1 | (100%) |
| IEY407 | BY | RM | S. par | 2/3 | (66%) | 2/2 | (100%) |
| IEY408 | BY | S. par | BY | 2/3 | (66%) | 2/2 | (100%) |
| IEY409 | BY | S. par | RM | 3/3 | (100%) | 3/3 | (100%) |
| IEY410 | BY | S. par | S. par | 2/3 | (66%) | 2/2 | (100%) |
| IEY411 | S. par | BY | S. par | 3/3 | (100%) | 2/3 | (66%) |
| IEY412 | S. par | BY | RM | 2/3 | (66%) | 2/2 | (100%) |
| IEY413 | S. par | BY | BY | 2/3 | (66%) | 1/2 | (50%) |
| IEY414 | S. par | S. par | BY | 2/3 | (66%) | 2/2 | (100%) |
| IEY415 | S. par | RM | BY | 2/3 | (66%) | 2/2 | (100%) |
| IEY416 | RM | BY | BY | 1/3 | (33%) | 1/1 | (100%) |
| IEY417 | RM | BY | RM | 2/3 | (66%) | 2/2 | (100%) |
| IEY418 | RM | BY | S. par | 2/3 | (66%) | 1/2 | (50%) |
| IEY419 | RM | RM | BY | 1/3 | (33%) | 1/1 | (100%) |
| IEY420 | RM | S. par | BY | 2/3 | (66%) | 2/2 | (100%) |

Column 1 (‘Strain’) lists the ID of all the strains containing chimeric versions of ChrI. Column 2-4 (‘Segment1’, ‘Segment2’ and ‘Segment3’) lists the strain specific segment associated to each chimera ChrI in distinct chimera strains. Column 5 (‘Assembly efficiency’) lists the efficiency of assembling the synthetic ChrI for each chimera cell based on PCR checking of 5 junctions across the assembled ChrI. Column 6 (‘Native ChrI elimination efficiency’) lists the efficiency of elimination of native BY ChrI based on PCR checking of segments covering the native ChrI.

TABLE 7
Phenotypic assay of all BY ChrI chimera strains growing
on rich media at distinct temperature, 30 and 35°
C. Column I (‘Strain’) lists IDs of each chimera strain.

| | | | | Doubling | Doubling |
|---|---|---|---|---|---|
| | | | | time at | time at |
| Strain | Segment 1 | Segment 2 | Segment 3 | 30° C. (min) | 35° C. (min) |
| IEY394 | S. par | S. par | S. par | 111.44 | 165.08 |
| IEY394 | S. par | S. par | S. par | 134.50 | 149.37 |
| IEY394 | S. par | S. par | S. par | 108.43 | 140.56 |
| IEY394 | S. par | S. par | S. par | 171.57 | 141.03 |
| IEY394 | S. par | S. par | S. par | 107.16 | 146.18 |
| IEY394 | S. par | S. par | S. par | 108.88 | 138.49 |
| IEY394 | S. par | S. par | S. par | 96.88 | 137.65 |
| IEY394 | S. par | S. par | S. par | 90.21 | 132.13 |
| IEY394 | S. par | S. par | S. par | 90.86 | 141.55 |
| IEY394 | S. par | S. par | S. par | 109.07 | 140.61 |
| IEY394 | S. par | S. par | S. par | 112.41 | 133.81 |
| IEY394 | S. par | S. par | S. par | 113.71 | 131.89 |
| IEY395 | S. par | RM | S. par | 115.39 | 129.06 |
| IEY395 | S. par | RM | S. par | 117.54 | 114.86 |
| IEY395 | S. par | RM | S. par | 114.86 | 114.20 |
| IEY395 | S. par | RM | S. par | 153.70 | 113.91 |
| IEY395 | S. par | RM | S. par | 106.71 | 116.01 |
| IEY395 | S. par | RM | S. par | 114.86 | 116.22 |
| IEY395 | S. par | RM | S. par | 94.59 | 109.02 |
| IEY395 | S. par | RM | S. par | 92.16 | 132.85 |
| IEY395 | S. par | RM | S. par | 93.07 | 113.63 |
| IEY395 | S. par | RM | S. par | 107.01 | 115.22 |
| IEY395 | S. par | RM | S. par | 152.05 | 112.60 |
| IEY395 | S. par | RM | S. par | 107.78 | 111.27 |
| IEY396 | S. par | S. par | RM | 141.48 | 100.33 |
| IEY396 | S. par | S. par | RM | 101.89 | 105.56 |
| IEY396 | S. par | S. par | RM | 97.58 | 97.22 |
| IEY396 | S. par | S. par | RM | 128.94 | 101.20 |
| IEY396 | S. par | S. par | RM | 101.69 | 100.53 |
| IEY396 | S. par | S. par | RM | 97.24 | 93.82 |
| IEY396 | S. par | S. par | RM | 84.27 | 109.88 |
| IEY396 | S. par | S. par | RM | 78.61 | 110.05 |
| IEY396 | S. par | S. par | RM | 78.55 | 113.75 |
| IEY396 | S. par | S. par | RM | 107.67 | 99.85 |
| IEY396 | S. par | S. par | RM | 108.14 | 96.66 |
| IEY396 | S. par | S. par | RM | 110.76 | 94.51 |
| IEY397 | S. par | RM | RM | 163.53 | 92.15 |
| IEY397 | S. par | RM | RM | 127.22 | 109.96 |
| IEY397 | S. par | RM | RM | 106.07 | 96.31 |
| IEY397 | S. par | RM | RM | 100.38 | 97.90 |
| IEY397 | S. par | RM | RM | 106.61 | 99.11 |
| IEY397 | S. par | RM | RM | 101.13 | 102.54 |
| IEY397 | S. par | RM | RM | 82.23 | 91.80 |
| IEY397 | S. par | RM | RM | 80.79 | 94.46 |
| IEY397 | S. par | RM | RM | 78.43 | 98.25 |
| IEY397 | S. par | RM | RM | 89.65 | 95.67 |
| IEY397 | S. par | RM | RM | 103.43 | 106.20 |
| IEY397 | S. par | RM | RM | 107.11 | 96.06 |
| IEY398 | RM | RM | RM | 193.21 | 88.70 |
| IEY398 | RM | RM | RM | 106.67 | 110.05 |
| IEY398 | RM | RM | RM | 97.48 | 91.66 |
| IEY398 | RM | RM | RM | 120.40 | 93.87 |
| IEY398 | RM | RM | RM | 108.35 | 99.16 |
| IEY398 | RM | RM | RM | 96.34 | 96.00 |
| IEY398 | RM | RM | RM | 79.69 | 116.07 |
| IEY398 | RM | RM | RM | 86.30 | 125.08 |
| IEY398 | RM | RM | RM | 78.76 | 114.31 |
| IEY398 | RM | RM | RM | 107.01 | 94.95 |
| IEY398 | RM | RM | RM | 131.56 | 104.81 |
| IEY398 | RM | RM | RM | 109.91 | 90.97 |
| IEY399 | RM | S. par | RM | 114.74 | 110.19 |
| IEY399 | RM | S. par | RM | 116.44 | 112.38 |
| IEY399 | RM | S. par | RM | 109.13 | 107.33 |
| IEY399 | RM | S. par | RM | 115.76 | 113.84 |
| IEY399 | RM | S. par | RM | 102.88 | 111.43 |
| IEY399 | RM | S. par | RM | 107.20 | 113.95 |
| IEY399 | RM | S. par | RM | 94.40 | 120.90 |
| IEY399 | RM | S. par | RM | 92.74 | 120.19 |
| IEY399 | RM | S. par | RM | 86.28 | 119.08 |
| IEY399 | RM | S. par | RM | 112.55 | 107.46 |
| IEY399 | RM | S. par | RM | 123.36 | 106.58 |
| IEY399 | RM | S. par | RM | 119.01 | 103.29 |
| IEY400 | RM | RM | S. par | 103.70 | 91.70 |
| IEY400 | RM | RM | S. par | 100.39 | 89.97 |
| IEY400 | RM | RM | S. par | 103.55 | 87.86 |
| IEY400 | RM | RM | S. par | 101.79 | 99.19 |
| IEY400 | RM | RM | S. par | 97.95 | 97.10 |
| IEY400 | RM | RM | S. par | 108.34 | 97.16 |
| IEY400 | RM | RM | S. par | 84.52 | 90.03 |
| IEY400 | RM | RM | S. par | 81.97 | 92.55 |
| IEY400 | RM | RM | S. par | 81.09 | 95.57 |
| IEY400 | RM | RM | S. par | 97.74 | 94.86 |
| IEY400 | RM | RM | S. par | 94.87 | 93.30 |
| IEY400 | RM | RM | S. par | 95.65 | 96.17 |
| IEY401 | RM | S. par | S. par | 96.34 | 105.44 |
| IEY401 | RM | S. par | S. par | 111.43 | 102.98 |
| IEY401 | RM | S. par | S. par | 104.02 | 105.55 |
| IEY401 | RM | S. par | S. par | 122.67 | 105.08 |
| IEY401 | RM | S. par | S. par | 123.57 | 113.44 |
| IEY401 | RM | S. par | S. par | 99.25 | 103.67 |
| IEY401 | RM | S. par | S. par | 83.59 | 153.78 |
| IEY401 | RM | S. par | S. par | 79.26 | 131.62 |
| IEY401 | RM | S. par | S. par | 82.72 | 144.71 |
| IEY401 | RM | S. par | S. par | 105.90 | 126.25 |
| IEY401 | RM | S. par | S. par | 108.08 | 114.88 |
| IEY401 | RM | S. par | S. par | 110.53 | 121.76 |
| IEY402 | BY | BY | BY | 100.73 | 93.58 |
| IEY402 | BY | BY | BY | 134.91 | 91.15 |
| IEY402 | BY | BY | BY | 98.83 | 91.61 |
| IEY402 | BY | BY | BY | 98.59 | 99.59 |
| IEY402 | BY | BY | BY | 107.57 | 99.90 |
| IEY402 | BY | BY | BY | 97.05 | 99.31 |
| IEY402 | BY | BY | BY | 79.31 | 94.74 |
| IEY402 | BY | BY | BY | 79.51 | 99.55 |
| IEY402 | BY | BY | BY | 82.44 | 96.03 |
| IEY402 | BY | BY | BY | 97.04 | 99.24 |
| IEY402 | BY | BY | BY | 100.13 | 96.04 |
| IEY402 | BY | BY | BY | 94.55 | 94.92 |
| IEY403 | BY | BY | RM | 104.38 | 95.21 |
| IEY403 | BY | BY | RM | 98.45 | 89.49 |
| IEY403 | BY | BY | RM | 98.11 | 93.03 |
| IEY403 | BY | BY | RM | 97.57 | 98.20 |
| IEY403 | BY | BY | RM | 95.08 | 97.17 |
| IEY403 | BY | BY | RM | 96.55 | 97.95 |
| IEY403 | BY | BY | RM | 80.10 | 98.51 |
| IEY403 | BY | BY | RM | 78.02 | 91.58 |
| IEY403 | BY | BY | RM | 79.91 | 100.46 |
| IEY403 | BY | BY | RM | 98.98 | 95.35 |
| IEY403 | BY | BY | RM | 96.09 | 97.72 |
| IEY403 | BY | BY | RM | 92.36 | 95.99 |
| IEY404 | BY | BY | S. par | 100.57 | 89.86 |
| IEY404 | BY | BY | S. par | 95.16 | 92.63 |
| IEY404 | BY | BY | S. par | 105.89 | 92.34 |
| IEY404 | BY | BY | S. par | 95.67 | 99.15 |
| IEY404 | BY | BY | S. par | 84.81 | 93.95 |
| IEY404 | BY | BY | S. par | 141.96 | 97.09 |
| IEY404 | BY | BY | S. par | 77.73 | 96.28 |
| IEY404 | BY | BY | S. par | 74.52 | 92.79 |
| IEY404 | BY | BY | S. par | 83.45 | 89.94 |
| IEY404 | BY | BY | S. par | 96.24 | 111.71 |
| IEY404 | BY | BY | S. par | 98.27 | 111.13 |
| IEY404 | BY | BY | S. par | 96.87 | 113.70 |
| IEY405 | BY | RM | BY | 150.58 | 89.89 |
| IEY405 | BY | RM | BY | 97.66 | 93.34 |
| IEY405 | BY | RM | BY | 94.45 | 88.26 |
| IEY405 | BY | RM | BY | 135.82 | 95.63 |
| IEY405 | BY | RM | BY | 95.62 | 93.16 |
| IEY405 | BY | RM | BY | 99.28 | 91.42 |
| IEY405 | BY | RM | BY | 83.12 | 100.11 |
| IEY405 | BY | RM | BY | 73.78 | 96.78 |
| IEY405 | BY | RM | BY | 74.79 | 96.21 |
| IEY405 | BY | RM | BY | 99.23 | 92.95 |
| IEY405 | BY | RM | BY | 98.31 | 99.88 |
| IEY405 | BY | RM | BY | 97.62 | 96.07 |
| IEY406 | BY | RM | RM | 98.64 | 90.42 |
| IEY406 | BY | RM | RM | 99.96 | 97.39 |
| IEY406 | BY | RM | RM | 103.43 | 92.24 |
| IEY406 | BY | RM | RM | 97.69 | 98.47 |
| IEY406 | BY | RM | RM | 98.96 | 98.65 |
| IEY406 | BY | RM | RM | 107.56 | 106.07 |
| IEY406 | BY | RM | RM | 81.10 | 98.12 |
| IEY406 | BY | RM | RM | 80.45 | 95.23 |
| IEY406 | BY | RM | RM | 86.00 | 99.96 |
| IEY406 | BY | RM | RM | 97.24 | 91.73 |
| IEY406 | BY | RM | RM | 98.77 | 95.17 |
| IEY406 | BY | RM | RM | 107.68 | 96.90 |
| IEY407 | BY | RM | S. par | 103.71 | 114.27 |
| IEY407 | BY | RM | S. par | 108.85 | 111.31 |
| IEY407 | BY | RM | S. par | 107.67 | 107.95 |
| IEY407 | BY | RM | S. par | 103.68 | 111.91 |
| IEY407 | BY | RM | S. par | 109.95 | 111.00 |
| IEY407 | BY | RM | S. par | 103.46 | 114.86 |
| IEY407 | BY | RM | S. par | 80.30 | 150.23 |
| IEY407 | BY | RM | S. par | 85.17 | 159.99 |
| IEY407 | BY | RM | S. par | 86.51 | 148.75 |
| IEY407 | BY | RM | S. par | 150.37 | 114.09 |
| IEY407 | BY | RM | S. par | 155.89 | 110.30 |
| IEY407 | BY | RM | S. par | 151.36 | 115.41 |
| IEY408 | BY | S. par | BY | 90.42 | 85.75 |
| IEY408 | BY | S. par | BY | 94.23 | 84.32 |
| IEY408 | BY | S. par | BY | 103.81 | 83.90 |
| IEY408 | BY | S. par | BY | 90.35 | 86.98 |
| IEY408 | BY | S. par | BY | 92.88 | 89.61 |
| IEY408 | BY | S. par | BY | 91.66 | 87.60 |
| IEY408 | BY | S. par | BY | 73.21 | 86.86 |
| IEY408 | BY | S. par | BY | 76.60 | 86.56 |
| IEY408 | BY | S. par | BY | 74.02 | 82.62 |
| IEY408 | BY | S. par | BY | 90.13 | 86.21 |
| IEY408 | BY | S. par | BY | 90.40 | 87.49 |
| IEY408 | BY | S. par | BY | 90.65 | 88.90 |
| IEY409 | BY | S. par | RM | 95.71 | 80.46 |
| IEY409 | BY | S. par | RM | 96.66 | 84.94 |
| IEY409 | BY | S. par | RM | 97.24 | 84.59 |
| IEY409 | BY | S. par | RM | 111.19 | 89.25 |
| IEY409 | BY | S. par | RM | 93.64 | 88.38 |
| IEY409 | BY | S. par | RM | 94.55 | 89.38 |
| IEY409 | BY | S. par | RM | 74.41 | 85.89 |
| IEY409 | BY | S. par | RM | 74.02 | 86.26 |
| IEY409 | BY | S. par | RM | 76.05 | 89.58 |
| IEY409 | BY | S. par | RM | 93.41 | 88.87 |
| IEY409 | BY | S. par | RM | 90.63 | 87.04 |
| IEY409 | BY | S. par | RM | 96.19 | 89.56 |
| IEY410 | BY | S. par | S. par | 99.23 | 92.58 |
| IEY410 | BY | S. par | S. par | 99.32 | 93.79 |
| IEY410 | BY | S. par | S. par | 97.82 | 91.94 |
| IEY410 | BY | S. par | S. par | 96.19 | 97.97 |
| IEY410 | BY | S. par | S. par | 106.00 | 97.81 |
| IEY410 | BY | S. par | S. par | 98.09 | 94.37 |
| IEY410 | BY | S. par | S. par | 77.93 | 96.06 |
| IEY410 | BY | S. par | S. par | 78.50 | 96.48 |
| IEY410 | BY | S. par | S. par | 81.09 | 98.55 |
| IEY410 | BY | S. par | S. par | 95.59 | 94.81 |
| IEY410 | BY | S. par | S. par | 91.86 | 94.21 |
| IEY410 | BY | S. par | S. par | 91.11 | 95.25 |
| IEY411 | S. par | BY | S. par | 139.00 | 106.99 |
| IEY411 | S. par | BY | S. par | 105.68 | 109.25 |
| IEY411 | S. par | BY | S. par | 103.82 | 108.37 |
| IEY411 | S. par | BY | S. par | 103.24 | 119.22 |
| IEY411 | S. par | BY | S. par | 103.22 | 109.96 |
| IEY411 | S. par | BY | S. par | 103.19 | 109.30 |
| IEY411 | S. par | BY | S. par | 83.73 | 113.16 |
| IEY411 | S. par | BY | S. par | 89.59 | 109.83 |
| IEY411 | S. par | BY | S. par | 87.08 | 97.96 |
| IEY411 | S. par | BY | S. par | 103.48 | 104.34 |
| IEY411 | S. par | BY | S. par | 96.79 | 107.79 |
| IEY411 | S. par | BY | S. par | 101.25 | 108.55 |
| IEY412 | S. par | BY | RM | 95.21 | 90.40 |
| IEY412 | S. par | BY | RM | 93.95 | 91.75 |
| IEY412 | S. par | BY | RM | 105.71 | 87.36 |
| IEY412 | S. par | BY | RM | 92.19 | 96.81 |
| IEY412 | S. par | BY | RM | 94.98 | 95.19 |
| IEY412 | S. par | BY | RM | 112.75 | 94.28 |
| IEY412 | S. par | BY | RM | 74.02 | 90.03 |
| IEY412 | S. par | BY | RM | 74.32 | 91.88 |
| IEY412 | S. par | BY | RM | 76.20 | 85.32 |
| IEY412 | S. par | BY | RM | 87.36 | 90.15 |
| IEY412 | S. par | BY | RM | 90.35 | 89.91 |
| IEY412 | S. par | BY | RM | 94.10 | 92.06 |
| IEY413 | S. par | BY | BY | 125.41 | 92.98 |
| IEY413 | S. par | BY | BY | 102.03 | 91.16 |
| IEY413 | S. par | BY | BY | 97.33 | 98.29 |
| IEY413 | S. par | BY | BY | 95.77 | 97.89 |
| IEY413 | S. par | BY | BY | 113.60 | 100.17 |
| IEY413 | S. par | BY | BY | 98.10 | 99.37 |
| IEY413 | S. par | BY | BY | 78.05 | 100.01 |
| IEY413 | S. par | BY | BY | 77.70 | 92.95 |
| IEY413 | S. par | BY | BY | 80.50 | 97.17 |
| IEY413 | S. par | BY | BY | 108.41 | 93.21 |
| IEY413 | S. par | BY | BY | 91.55 | 91.25 |
| IEY413 | S. par | BY | BY | 98.40 | 93.03 |
| IEY414 | S. par | S. par | BY | 109.75 | 115.35 |
| IEY414 | S. par | S. par | BY | 107.50 | 116.79 |
| IEY414 | S. par | S. par | BY | 181.90 | 107.08 |
| IEY414 | S. par | S. par | BY | 107.29 | 115.33 |
| IEY414 | S. par | S. par | BY | 108.19 | 113.85 |
| IEY414 | S. par | S. par | BY | 141.57 | 110.15 |
| IEY414 | S. par | S. par | BY | 83.85 | 125.41 |
| IEY414 | S. par | S. par | BY | 85.84 | 110.89 |
| IEY414 | S. par | S. par | BY | 91.67 | 114.56 |
| IEY414 | S. par | S. par | BY | 102.61 | 96.99 |
| IEY414 | S. par | S. par | BY | 105.53 | 99.04 |
| IEY414 | S. par | S. par | BY | 137.91 | 98.52 |
| IEY415 | S. par | RM | BY | 100.30 | 96.59 |
| IEY415 | S. par | RM | BY | 101.86 | 94.89 |
| IEY415 | S. par | RM | BY | 110.77 | 91.46 |
| IEY415 | S. par | RM | BY | 99.98 | 98.98 |
| IEY415 | S. par | RM | BY | 99.53 | 99.53 |
| IEY415 | S. par | RM | BY | 110.56 | 98.24 |
| IEY415 | S. par | RM | BY | 79.62 | 93.86 |
| IEY415 | S. par | RM | BY | 83.55 | 98.18 |
| IEY415 | S. par | RM | BY | 83.43 | 93.47 |
| IEY415 | S. par | RM | BY | 93.84 | 98.54 |
| IEY415 | S. par | RM | BY | 100.18 | 98.69 |
| IEY415 | S. par | RM | BY | 96.10 | 98.00 |
| IEY416 | RM | BY | BY | 102.25 | 91.42 |
| IEY416 | RM | BY | BY | 99.58 | 88.05 |
| IEY416 | RM | BY | BY | 101.93 | 88.97 |
| IEY416 | RM | BY | BY | 99.51 | 96.98 |
| IEY416 | RM | BY | BY | 96.00 | 94.96 |
| IEY416 | RM | BY | BY | 99.08 | 96.30 |
| IEY416 | RM | BY | BY | 80.06 | 93.04 |
| IEY416 | RM | BY | BY | 81.26 | 88.82 |
| IEY416 | RM | BY | BY | 83.34 | 93.87 |
| IEY416 | RM | BY | BY | 93.37 | 91.22 |
| IEY416 | RM | BY | BY | 92.10 | 94.51 |
| IEY416 | RM | BY | BY | 95.75 | 88.39 |
| IEY416 | RM | BY | S. par | 100.16 | 91.48 |
| IEY418 | RM | BY | S. par | 97.36 | 93.09 |
| IEY418 | RM | BY | S. par | 99.78 | 91.39 |
| IEY418 | RM | BY | S. par | 98.90 | 98.07 |
| IEY418 | RM | BY | S. par | 99.47 | 97.66 |
| IEY418 | RM | BY | S. par | 123.89 | 94.00 |
| IEY418 | RM | BY | S. par | 80.23 | 96.26 |
| IEY418 | RM | BY | S. par | 79.48 | 95.25 |
| IEY418 | RM | BY | S. par | 78.48 | 98.38 |
| IEY418 | RM | BY | S. par | 95.93 | 94.61 |
| IEY418 | RM | BY | S. par | 93.44 | 93.98 |
| IEY418 | RM | BY | S. par | 97.87 | 95.14 |
| IEY419 | RM | RM | BY | 103.16 | 110.37 |
| IEY419 | RM | RM | BY | 99.66 | 106.30 |
| IEY419 | RM | RM | BY | 100.88 | 104.78 |
| IEY419 | RM | RM | BY | 99.60 | 118.36 |
| IEY419 | RM | RM | BY | 100.01 | 112.71 |
| IEY419 | RM | RM | BY | 123.84 | 116.38 |
| IEY419 | RM | RM | BY | 81.33 | 100.52 |
| IEY419 | RM | RM | BY | 81.48 | 103.17 |
| IEY419 | RM | RM | BY | 77.98 | 106.82 |
| IEY419 | RM | RM | BY | 97.73 | 102.41 |
| IEY419 | RM | RM | BY | 93.84 | 106.34 |
| IEY419 | RM | RM | BY | 99.22 | 110.66 |
| IEY420 | RM | S. par | BY | 98.97 | 93.49 |
| IEY420 | RM | S. par | BY | 102.52 | 88.93 |
| IEY420 | RM | S. par | BY | 96.82 | 95.47 |
| IEY420 | RM | S. par | BY | 101.67 | 97.57 |
| IEY420 | RM | S. par | BY | 105.74 | 98.14 |
| IEY420 | RM | S. par | BY | 98.89 | 98.90 |
| IEY420 | RM | S. par | BY | 83.19 | 93.09 |
| IEY420 | RM | S. par | BY | 82.34 | 94.19 |
| IEY420 | RM | S. par | BY | 81.41 | 97.76 |
| IEY420 | RM | S. par | BY | 92.21 | 94.96 |
| IEY420 | RM | S. par | BY | 91.60 | 96.19 |
| IEY420 | RM | S. par | BY | 93.95 | 99.79 |

Column 2 through 4 (‘Segment1’, ‘Segment2’ and ‘Segment3’) lists each strain-specific segment to its respective chimera strain. Column 5 (‘Doubling time at 30° C. (min)’) lists the doubling time of each chimera strain growing on rich media at 30° C. Column 5 (‘Doubling time at 35° C. (min)’) lists the doubling time of each chimera strain growing on rich media at 35° C.

TABLE 8
Full factorial ANOVA table for growth of chimera ChrI cells at 35°
C. PVE (phenotypic variance explained). Interaction terms are denoted by ‘;’.

| | | Sum | Mean | F | | |
|---|---|---|---|---|---|---|
| | Df | Sq | Sq | value | Pr(>F) | PVE(%) |
| Segment 1 | 2 | 1.539 | 0.7694 | 52.452 | <2e−16 *** | 8.9 |
| Segment 2 | 2 | 1.085 | 0.5425 | 36.985 | 5.18e−15 *** | 6.3 |
| Segment 3 | 2 | 3.784 | 1.8922 | 129.003 | <2e−16 *** | 22 |
| Segment1; Segment2 | 4 | 3.594 | 0.8985 | 61.256 | <2e−16 *** | 20 |
| Segment1; Segment3 | 4 | 1.253 | 0.3132 | 21.355 | 1.99e−15 *** | 7.3 |
| Segment2; Segment3 | 4 | 0.314 | 0.0786 | 5.365 | 0.000358 *** | 1.8 |
| Segment1; | 7 | 1.394 | 0.1992 | 13.579 | 3.76e−15 *** | 8.1 |
| Segment2; Segment3 | | | | | | |
| Residuals | 286 | 4.195 | 0.0147 | | | |

TABLE 9
Efficiency of assembly of synthetic restructured
ChrI in BY and elimination of its native ChrI.

| | | | | | | Native ChrI | |
|---|---|---|---|---|---|---|---|
| | Position | Position | Position | Assembly | | elimination | |
| Strain | 1 | 2 | 3 | efficiency | | efficiency | |
| IEY421 | Seg1 | Seg3 | Seg2 | 1/3 | (33%) | 1/1 | (100%) |
| IEY422 | Seg2 | Seg1 | Seg3 | 3/3 | (100%) | 2/3 | (66%) |
| IEY423 | Seg2 | Seg3 | Seg1 | 3/3 | (100%) | 3/3 | (100%) |
| IEY424 | Seg3 | Seg2 | Seg1 | 3/3 | (100%) | 3/3 | (100%) |
| IEY425 | Seg3 | Seg1 | Seg2 | 2/3 | (66%) | 2/2 | (100%) |

Column 1 (‘Strain’) lists the ID of all the strains containing restructured versions of ChrI. Column 2-4 (‘Position1’, ‘Position2’ and ‘Position3’) lists the order of assembled segments for each restructured version of chromosome I. Column 5 (‘Assembly efficiency’) lists the efficiency of assembling the restructured ChrI for each strain cell based on PCR checking of 5 junctions across the assembled ChrI. Column 6 (‘Native ChrI elimination efficiency’) lists the efficiency of elimination of native BY ChrI based on PCR checking of segments covering the native ChrI. A total of 12 replicas per strain were phenotyped on both conditions.

TABLE 10
Phenotypic assay of strains containing
the restructured versions of BY ChrI.

| Strain | Position 1 | Position 2 | Position 3 | Doubling time |
|---|---|---|---|---|
| Synthetic ChrI | Seg1 | Seg2 | Seg3 | 96.62 |
| Synthetic ChrI | Seg1 | Seg2 | Seg3 | 93.60 |
| Synthetic ChrI | Seg1 | Seg2 | Seg3 | 94.60 |
| Synthetic ChrI | Seg1 | Seg2 | Seg3 | 90.30 |
| Synthetic ChrI | Seg1 | Seg2 | Seg3 | 92.72 |
| Synthetic ChrI | Seg1 | Seg2 | Seg3 | 92.21 |
| Synthetic ChrI | Seg1 | Seg2 | Seg3 | 91.19 |
| Synthetic ChrI | Seg1 | Seg2 | Seg3 | 92.23 |
| Synthetic ChrI | Seg1 | Seg2 | Seg3 | 87.94 |
| Synthetic ChrI | Seg1 | Seg2 | Seg3 | 92.46 |
| Synthetic ChrI | Seg1 | Seg2 | Seg3 | 93.59 |
| Synthetic ChrI | Seg1 | Seg2 | Seg3 | 87.00 |
| IEY421 | Seg1 | Seg3 | Seg2 | 159.29 |
| IEY421 | Seg1 | Seg3 | Seg2 | 154.82 |
| IEY421 | Seg1 | Seg3 | Seg2 | 154.62 |
| IEY421 | Seg1 | Seg3 | Seg2 | 158.20 |
| IEY421 | Seg1 | Seg3 | Seg2 | 152.05 |
| IEY421 | Seg1 | Seg3 | Seg2 | 154.72 |
| IEY421 | Seg1 | Seg3 | Seg2 | 172.11 |
| IEY421 | Seg1 | Seg3 | Seg2 | 159.60 |
| IEY421 | Seg1 | Seg3 | Seg2 | 154.00 |
| IEY421 | Seg1 | Seg3 | Seg2 | 161.71 |
| IEY421 | Seg1 | Seg3 | Seg2 | 155.03 |
| IEY421 | Seg1 | Seg3 | Seg2 | 152.42 |
| IEY422 | Seg2 | Seg1 | Seg3 | 94.50 |
| IEY422 | Seg2 | Seg1 | Seg3 | 91.72 |
| IEY422 | Seg2 | Seg1 | Seg3 | 92.10 |
| IEY422 | Seg2 | Seg1 | Seg3 | 100.66 |
| IEY422 | Seg2 | Seg1 | Seg3 | 94.66 |
| IEY422 | Seg2 | Seg1 | Seg3 | 92.03 |
| IEY422 | Seg2 | Seg1 | Seg3 | 95.01 |
| IEY422 | Seg2 | Seg1 | Seg3 | 89.22 |
| IEY422 | Seg2 | Seg1 | Seg3 | 93.12 |
| IEY422 | Seg2 | Seg1 | Seg3 | 96.33 |
| IEY422 | Seg2 | Seg1 | Seg3 | 91.71 |
| IEY422 | Seg2 | Seg1 | Seg3 | 92.06 |
| IEY423 | Seg2 | Seg3 | Seg1 | 85.52 |
| IEY423 | Seg2 | Seg3 | Seg1 | 86.26 |
| IEY423 | Seg2 | Seg3 | Seg1 | 87.82 |
| IEY423 | Seg2 | Seg3 | Seg1 | 83.06 |
| IEY423 | Seg2 | Seg3 | Seg1 | 82.81 |
| IEY423 | Seg2 | Seg3 | Seg1 | 86.28 |
| IEY423 | Seg2 | Seg3 | Seg1 | 85.52 |
| IEY423 | Seg2 | Seg3 | Seg1 | 84.91 |
| IEY423 | Seg2 | Seg3 | Seg1 | 87.38 |
| IEY423 | Seg2 | Seg3 | Seg1 | 85.61 |
| IEY423 | Seg2 | Seg3 | Seg1 | 89.01 |
| IEY423 | Seg2 | Seg3 | Seg1 | 87.38 |
| IEY424 | Seg3 | Seg2 | Seg1 | 89.37 |
| IEY424 | Seg3 | Seg2 | Seg1 | 88.97 |
| IEY424 | Seg3 | Seg2 | Seg1 | 92.54 |
| IEY424 | Seg3 | Seg2 | Seg1 | 89.60 |
| IEY424 | Seg3 | Seg2 | Seg1 | 90.31 |
| IEY424 | Seg3 | Seg2 | Seg1 | 94.11 |
| IEY424 | Seg3 | Seg2 | Seg1 | 90.14 |
| IEY424 | Seg3 | Seg2 | Seg1 | 95.18 |
| IEY424 | Seg3 | Seg2 | Seg1 | 93.23 |
| IEY424 | Seg3 | Seg2 | Seg1 | 90.53 |
| IEY424 | Seg3 | Seg2 | Seg1 | 95.33 |
| IEY424 | Seg3 | Seg2 | Seg1 | 93.61 |
| IEY425 | Seg3 | Seg1 | Seg2 | 113.38 |
| IEY425 | Seg3 | Seg1 | Seg2 | 111.67 |
| IEY425 | Seg3 | Seg1 | Seg2 | 110.92 |
| IEY425 | Seg3 | Seg1 | Seg2 | 103.61 |
| IEY425 | Seg3 | Seg1 | Seg2 | 104.70 |
| IEY425 | Seg3 | Seg1 | Seg2 | 109.69 |
| IEY425 | Seg3 | Seg1 | Seg2 | 104.39 |
| IEY425 | Seg3 | Seg1 | Seg2 | 105.12 |
| IEY425 | Seg3 | Seg1 | Seg2 | 111.11 |
| IEY425 | Seg3 | Seg1 | Seg2 | 107.13 |
| IEY425 | Seg3 | Seg1 | Seg2 | 107.45 |
| IEY425 | Seg3 | Seg1 | Seg2 | 110.82 |

Column I (‘Strain’) lists IDs of each strain carrying a restructured version of ChrI. Column 2-4 (‘Position1’, ‘Position2’ and ‘Position3’) lists the order of assembled segments for each restructured version of chromosome I. Column 5 (‘Doubling time at 30° C. (min)’) lists the doubling time of each restructured ChrI strain growing on rich media at 30° C. A total of 9 replicas per strain were phenotyped.

TABLE 11
Cloning cassettes for chromosome I segment capture in Saccharomyces cerevisiae
BY4742 used for multiplex deletion. Each module contains a pair of upstream and downstream
adaptors flanking segment-specific homology arms that are separated by a site containing XhoI and
AvrII sites used for vector linearization. Column 1 (‘Cloning module’) lists the ID of each cloned
region. Column 2 (‘Sequence’) contains the nucleotide sequence of each specific clone module.

| Cloning | Sequence |
|---|---|
| cassette | |
| Segment 1 | gaattctttccgaatgacatgcgtctccttgcgggtaaatcaccgaccgcaattcatagaagcctgggggaacag |
| (SEQ ID NO: | ataggtctaattagcttaagagagtaaatcctcatatgtacatttatcgagccaatcgagggcagcagtttaac |
| 61) | atcaagccggatttgctcacgctactttgaccccttttcgtttcgacggagagaagaaaccggtgttttcctatc |
| | cttgcctctcgaggcgcgcatatcctaggtggtttcgatagaggccaagcgaataatactccttttccaggttattttg |
| | gaggtttggatttatttgattatgactttttgtttggcaatgactttgcttaaaaattttctttccaaactccagccgcgcta |
| | tccggtaatctccaaattaaaacataccgttccatgaaggctagaattacttaccggccttttccatgcctgcgctat |
| | acccccccactcgcatgc |
| Segment 2 | gaattcagccgcgctatccggtaatctccaaattaaaacataccgttccatgaaggctagaattacttaccggcct |
| (SEQ ID NO: | tttccatgcctgcgctatacccccccactcaaatgcaacacttccctaatatagaaatttgggcattaattatttt |
| 62) | gagaattttgatgatttgaataatttcattaacgtaaaggaacatagtgctacgaatccaacagtggacccaa |
| | aaatgactcgaggcgcgcatatcctaggatgcctgccactaggaatccatcgacgtaccatggctataactttcctta |
| | tgttgtttgcttagttttttgatattagtgttgcttatgtgaaatttcgcgatttcaattaaaataataaatacatatgcata |
| | tggtccacaggacactcgtcgctttcggatttgccctctatgtggcggttttcaggcacacttatgctcagcaccgttta |
| | aaccagaccgacgcatg |
| Segment 3 | gaattctatgcatatggtccacaggacactcgtcgctttcggatttgccctctatgtggcggttttcaggcacacttat |
| (SEQ ID NO: | gctcagcaccgtttaaaccagaccgacctttccagattacaagaactttgcgtttggcctctacaacgattctc |
| 63) | acaagcataagggccatgctggtgtacagggaaatgtctctgctgagacacatttccggattgagatggtca |
| | gtaaaactcgaggcgcgcatatcctaggttggtggtttaatctattttttataaaaaatgacgcgggcagattcaatta |
| | gtgtcctaaatttattcgcgtttcaagatttcaaaggattgatcctcttatcagaaacgataagtgctactccgaactacg |
| | atgccgctaagaacctctcggtcgtcgctgacgtttacactctagtctcattataatcgttcgctattcagggattga |
| | ccaacaccggaaaacgcatg |
| Segment 4 | gaattcaactacgatgccgctaagaacctctcggtcgtcgctgacgtttacactctagtctcattataatcgttcgct |
| (SEQ ID NO: | attcagggattgaccaacaccggaaaacttttttcatttcttatattattttttgttcgagaatcactttttcaagat |
| 64) | ggtaacaacatcttcgtcttccaaaatgtgactcaaccccacgtattgaggttgatgtttgacactgctaccgt |
| | actcgaggcgcgcatatcctaggtggtacaaggatctaaaaatgaagatgtgtctggctttagtaatcatcatattgct |
| | tgttgtaatcatcgtccccattgctgttcactttagtcgatagaagttcactcgcaatgctatatatatgtcactagagga |
| | cgcacgctctatttttatgatccattgatgtccctgacgctgcaaaatttgcaaccaggcagtcttcgcggtaggtc |
| | ctagtgcaatggcatgc |
| Segment 5 | gaattcgtcactagaggacgcacgctctatttttatgatccattgatgtccctgacgctgcaaaatttgcaaccagg |
| (SEQ ID NO: | cagtcttcgcggtaggtcctagtgcaatgaggaattgagtatgcagaatcagaataaaggctgactttcaaaaaaag |
| 65) | gttgtattacaattgcaggttttcgataaaagagaccctattctcatctactactgctaaacttcgagatattttcgctcga |
| | ggcgcgcatatcctaggctgttgctgttgctgctgcgggcccacattagggtagcctagtaaagaagggtcgttcatt |
| | agcggtagtttgcagggataacgtcagtcggagttcccttgctggtgtcccttatgtgtgcccatactcttccataaa |
| | cgggctattagttatgaggtccgaagattgaaaaaggtgagggaactcggccgaacgggaaagacggacat |
| | ctaggcaacctgacgcatgc |
| Segment 6 | gaattcatactcttccataaacgggctattagttatgaggtccgaagattgaaaaaggtgagggaactcggccg |
| (SEQ ID NO: | aacgggaaagacggacatctaggcaacctgactggcgtgtgctatagtgctctatattcgagtttgttgctact |
| 66) | ggtggacacccgactatctacagtaaggaacgtaaacaagaaaaagagagaaaatacgctatagttgaaa |
| | acatgagtggttcctcgaggcgcgcatatcctaggttgtttgattttggtccttttctccggtaggagttctgattctgg |
| | cccagtttcagtctttaccagcggtcttttcctcagaattgccatagatgagtatttactgatcttttgcatatttttttgtc |
| | gctcttgggccgcggtgcgctaccttgcaggaattgagaccgtccgttaatttcccttgcatatatattgcgtttctttga |
| | ccttttaaccgctctcgcatgc |
| Segment 7 | gaattgtcgctcttgggccgcggtgcgctaccttgcaggaattgagaccgtccgttaatttcccttgcatatatattg |
| (SEQ ID NO: | cgtttctttgaccttttaaccgctctccggcaaagtacaagggaaggaagcacagaagcaagaggaggcgcat |
| 67) | cgatcgtggcagatgagtcagcaaacaccacaggaaagtgaacagaccacagcgaaagaacaggacctt |
| | gatcaagagagctcgaggcgcgcatatcctaggactcataatatcttgtcgaaaatacgcggtgtagggagttatg |
| | gtggataactttttcacgattagaagaaaaggaaaatttcattattcgtagcttaacatggcaaaaacgagaaagacat |
| | atacaaaagcttaaatgggaaatacgcgcccataacttggtgcgaatacgggtcgtagcaatgttcgtctgacta |
| | tgatctacatattacaggcggtacgtctgcatgc |
| Segment 8 | gaattccaaaagcttaaatgggaaatacgcgcccataacttggtgcgaatacgggtcgtagcaatgttcgtctga |
| (SEQ ID NO: | ctatgatctacatattacaggcggtacgtctcatgaagcgaaagtacttcacgacacctagattgcaatctactc |
| 68) | aatgttatccctggatgaaatattatttcgttaacgaccatagtaactacctgcttccatatgtttggcctaatg |
| | gaaccactcgaggcgcgcatatcctaggaatcgtcagtaccatttttcttgtggctagttggcttcaaccaaacgtcct |
| | cttctctcttatggcaagaagaaagttatatgtgtgactggttgtttatttcactttcgcgactgaaagcgccgtctaaac |
| | tagaaagttaccgttagtcttactgatccgaaccgtcattagttactagaggaaacgtgtcaagaatgccagccg |
| | atcccacattcttaattagcatgc |
| Segment 9 | gaattcaatcaaagggaagtcgaataaaggtcatccagggaattagccgttttcgaccaagtctttacgggagt |
| (SEQ ID NO: | agaagacgaatcctctcgtgtgctggcccaggaaattaagatagttattcgagtgattgccaaatatcatgttc |
| 69 | tacttcgaagacttatagctaattaattttttcataatgaaggtgtcgttaattgttctgattagtaacatgaaac |
| | tcaaaaactcgaggcgcgcatatcctaggagtagttgtttatcactagacatataattatgtttatttatatttagtggga |
| | gcaaaacagtttattgaatgtttaccagaaccgaaaaaaaagctcttctaaactgttgacatccagttcattttactaggt |
| | aactagaggatttgcgacgttctaagcgttggtccatgtgaatcgccatccaggatcacgtcgccctgaaaaaa |
| | agatatcagcaactctccgcatgc |
| Segment 10 | gaattctactaggtaactagaggatttgcgacgttctaagcgttggtccatgtgaatcgccatccaggatcacgtc |
| (SEQ ID NO: | gccctgaaaaaaagatatcagcaactctcctacccgtatgtattaatgtataaatgttctcagagcaaattttat |
| 70) | cgatatcttgtttgccagtggtatgcaggtttggcaaattttttaccataatatccgtttatagattctggaacctt |
| | accctcgaggcgcgcatatcctaggcctgaaaatttgaggttgttcacggaaatcatttggttatgtctgtcggcctgc |
| | tatttagagacattttttattgcaacaacctactctatgcacttacacggaatcgcagaataacgcgcccatgctctagg |
| | catctaactattcccactgccttaagggggcctgcgttttctgcctgtcgatccataggactcgtgtcaacgcgcag |
| | gcttagttcgagatgcatgc |
| Segment 11 | gaattctgctctaggcatctaactattcccactgccttaagggggcctgcgttttctgcctgtcgatccataggactc |
| (SEQ ID NO: | gtgtcaacgcgcaggcttagttcgagatgggtgtaaaagttatgtacgctcgaaacaaattttatgtagtttact |
| 71 | ttagatgcaaatgctattatatattttgctttatgatcctcggcttgatgctcgccaacgtgagatagctggtcat |
| | cactcgaggcgcgcatatcctagggacaataactggcaagggctctcactaaatatcaacccctttcaaataaaaaa |
| | aggatcatggctggcagcgcccacaacaattaaactctgttactatcaaaaaacattgagcccaagaatggaacttg |
| | atacctagttaacccacgggtgtggaaaagctccctaagcgccggaaggttttcctgagtgg |
| | cactcacacattatggtgtcaggcatgc |

Upstream adaptor (italicized); upstream homology arm (bold); downstream homology (underlined); and downstream adaptor (italicized and underlined)

TABLE 12
Location and size of each target segment on ChrI for
cloning or deletion during multiplex deletion assay.

| Segments | Start | End | Size (bp) |
|---|---|---|---|
| Deleted left sub-telomere | 1649 | 31266 | 29617 |
| Deleted segment 1 | 31267 | 34802 | 3535 |
| Cloned segment 1 | 34803 | 51727 | 16925 |
| Deleted segment 2 | 51728 | 52761 | 1033 |
| Cloned segment 2 | 52762 | 56949 | 4188 |
| Deleted segment 3 | 56950 | 57585 | 635 |
| Cloned segment 3 | 57586 | 73851 | 16266 |
| Deleted Segment 4 | 73852 | 75007 | 1155 |
| Cloned segment 4 | 75008 | 87778 | 12771 |
| Deleted segment 5 | 87779 | 92460 | 4681 |
| Cloned segment 5 | 92461 | 113424 | 20964 |
| Deleted segment 6 | 113425 | 114804 | 1379 |
| Cloned segment 6 | 114805 | 128110 | 13306 |
| Deleted segment 7 | 128111 | 129212 | 1101 |
| Cloned segment 7 | 129213 | 143552 | 14340 |
| Deleted segment 8 | 143553 | 147496 | 3943 |
| Cloned segment 8 | 147497 | 151278 | 3781 |
| Deleted Centromere | 151279 | 151900 | 621 |
| Cloned segment 9 | 151901 | 160045 | 8144 |
| Deleted segment 9 | 160046 | 170356 | 10310 |
| Cloned segment 10 | 170357 | 182386 | 12029 |
| Deleted segment 10 | 182387 | 184582 | 2195 |
| Cloned segment 11 | 184583 | 196952 | 12369 |
| Deleted right sub-telomere | 197029 | 229121 | 32092 |

Column 1 (‘Segment’) lists the IDs of all target segments. Column 2 (‘Start’) lists the start position of each segment referent to S. cerevisiae ChrI coordinates. Column 3 (‘End’) lists the end position for each segment. Column 4 (‘Size(bp)’) lists the size of each segment.

TABLE 13
Genetic elements removed from the ChrI multiple deletion strain.

| | Feature | | | |
|---|---|---|---|---|
| | Systematic | Feature | Coor- | |
| Feature | Name | Type | dinates | Location |
| GDH3 | YAL062W | ORF | 31567- | Deleted |
| | | | 32940 | segment 1 |
| BDH2 | YAL061W | ORF | 33448- | Deleted |
| | | | 34701 | segment 1 |
| AIM2 | YAL049C | ORF | 51855- | Deleted |
| | | | 52595 | segment 2 |
| BOL3 | YAL046C | ORF | 57029- | Deleted |
| | | | 57385 | segment 3 |
| YAL037W | YAL037W | ORF | 74020- | Deleted |
| | | | 74823 | segment 4 |
| MYO4 | YAL029C | ORF | 87855- | Deleted |
| | | | 92270 | segment 5 |
| ATS1 | YAL020C | ORF | 113614- | Deleted |
| | | | 114615 | segment 6 |
| SYN8 | YAL014C | ORF | 128252- | Deleted |
| | | | 129019 | segment 7 |
| VPS8 | YAL002W | ORF | 143707- | Deleted |
| | | | 147531 | segment 8 |
| YARCdelta3 | YARCdelta3 | Long | 160105- | Deleted |
| | | terminal | 160237 | segment 9 |
| | | repeat | | |
| YARCTy1-1 | YARCTy1-1 | LTR | 160238- | Deleted |
| | | retro- | 166162 | segment 9 |
| | | transposon | | |
| TGA1 | YNCA0004W | tRNA gene | 166267- | Deleted |
| | | | 166339 | segment 9 |
| BUD14 | YAR014C | ORF | 166742- | Deleted |
| | | | 168871 | segment 9 |
| ADE1 | YAR015W | ORF | 169375- | Deleted |
| | | | 170295 | segment 9 |
| YNCA0006C | YNCA0006C | tRNA gene | 182522- | Deleted |
| | | | 182603 | segment 10 |
| YARWsigma1 | YARWsigma1 | Long | 182620- | Deleted |
| | | terminal | 182959 | segment 10 |
| | | repeat | | |
| YARWdelta6 | YARWdelta6 | Long | 183142- | Deleted |
| | | terminal | 183474 | segment 10 |
| | | repeat | | |
| UIP3 | YAR027W | ORF | 183770- | Deleted |
| | | | 184477 | segment 10 |

Column 1 (‘Features’) lists the standard ID of each genetic element removed from ChrI. Column 2 (‘Systematic feature’) lists the systematic feature of each genetic element removed from ChrI. Column 3 (‘Feature type’) associates the type of feature to each genetic element. Column 4 (‘ Coordinates’) lists the start and end position of each removed element based on ChrI sequence coordinates. Column 5 (‘Location’) associates each removed genetic feature to a deleted segment into multiple deleted ChrI strain. A given deleted segment may contain one or more listed features.

TABLE 14
gRNAs used to clone segments for the multiplex deletion experiment.
Column 1 (‘gRNAs’) lists the gRNAs IDs. The ‘up’ and ‘down’
annotations refer to the side of a segment targeted by a
guide, Column 2 (‘sequence’) lists the target sequence of each gRNA.

| gRNAs | Sequence | SED ID NO |
|---|---|---|
| Segment 1 down | aattgatgaaatgaataggt | SEQ ID NO: 72 |
| Segment 1 down | atatatatatatgtatataa | SEQ ID NO: 73 |
| Segment 1 up | acgttctcgtgttaatcccg | SEQ ID NO: 74 |
| Segment 1 up | ttagtaaaacaagaagaccg | SEQ ID NO: 75 |
| Segment 10 down | caacacaattgggaaacgat | SEQ ID NO: 76 |
| Segment 10 down | aagcatactcatcttcaact | SEQ ID NO: 77 |
| Segment 10 up | tattcagtgaggagttacac | SEQ ID NO: 78 |
| Segment 10 up | tgtatgaatcatattcagtg | SEQ ID NO: 79 |
| Segment 11 up | gatggactgacacttaattg | SEQ ID NO: 80 |
| Segment 11 up | aattaagtgtcagtccatct | SEQ ID NO: 81 |
| Segment 11 down | ataaaattttcactacacct | SEQ ID NO: 82 |
| Segment 11 down | catgtacaaatccatgtccg | SEQ ID NO: 83 |
| Segment 2 down | tatataaagaatatacacag | SEQ ID NO: 84 |
| Segment 2 down | atataaagaatatacacaga | SEQ ID NO: 85 |
| Segment 2 up | tctgctgtagttagacgtag | SEQ ID NO: 86 |
| Segment 2 up | ctatgatatctggcctaagg | SEQ ID NO: 87 |
| Segment 3 down | agatggctagaagaatagga | SEQ ID NO: 88 |
| Segment 3 down | agatgggaagaaaactgaag | SEQ ID NO: 89 |
| Segment 3 up | atcctgaagagactggaatg | SEQ ID NO: 90 |
| Segment 3 up | gcattccagtctcttcagga | SEQ ID NO: 91 |
| Segment 4 down | ggagcttccatatgtagtgt | SEQ ID NO: 92 |
| Segment 4 down | catatatcctacactacata | SEQ ID NO: 93 |
| Segment 4 up | agcatttgtattcgaaaagg | SEQ ID NO: 94 |
| Segment 4 up | aagcatttgtattcgaaaag | SEQ ID NO: 95 |
| Segment 5 down | gagaaggttctcaagcacaa | SEQ ID NO: 96 |
| Segment 5 down | cgatgagaattaatgctgaa | SEQ ID NO: 97 |
| Segment 5 up | aaaaaaggtgataactccgt | SEQ ID NO: 98 |
| Segment 5 up | tgataactccgtaggaattg | SEQ ID NO: 99 |
| Segment 6 down | tatatgatgaatcattaaag | SEQ ID NO: 100 |
| Segment 6 down | atgatgaatcattaaagagg | SEQ ID NO: 101 |
| Segment 6 up | cagatccaaagcgcaagtga | SEQ ID NO: 102 |
| Segment 6 up | cacgtaacttaaatcagtac | SEQ ID NO: 103 |
| Segment 7 down | atcaaaacgtgagtttcctg | SEQ ID NO: 104 |
| Segment 7 down | aggtatatcatcgtaaccag | SEQ ID NO: 105 |
| Segment 7 up | cacgactttgatcacccgaa | SEQ ID NO: 106 |
| Segment 7 up | ataaactcataacagtgcaa | SEQ ID NO: 107 |
| Segment 8a down | ttatgatgcaaaaaaccaag | SEQ ID NO: 108 |
| Segment 8a down | ctgcattttcaaataccgct | SEQ ID NO: 109 |
| Segment 8a up | tgaatctacataatgaaatg | SEQ ID NO: 110 |
| Segment 8a up | aaatatcagaaatatgcaaa | SEQ ID NO: 111 |
| Segment 9 down | acttccacgtgtagatgtga | SEQ ID NO: 112 |
| Segment 9 down | tgttccttcacatctacacg | SEQ ID NO: 113 |
| Segment 9 up | cacctttcgtctgctgcctc | SEQ ID NO: 114 |
| Segment 9 up | ataatttgaaaaaacacccg | SEQ ID NO: 115 |

TABLE 15
Primers used to confirm capture of segments 1 through 11 on multiple deletion
assay. Column 1 (‘Primers’) lists primers IDs. Column 2 (‘Sequence’) contains
the nucleotide sequence of each primer.

| Primers | Sequence | SEQ ID NO |
|---|---|---|
| GDH3 check REV | ggaagcgtaggccaccct | SEQ ID NO: 116 |
| Seg1 capture confirm REV | gagccacaaatcccacacca | SEQ ID NO: 117 |
| Seg1 capture confirm FOR | agagcttggactacaacagtgg | SEQ ID NO: 118 |
| AIM2 check FOR | gagatgtagagcctgctgca | SEQ ID NO: 119 |
| Seg2 capture confirm REV | acccactgcacatatcatccag | SEQ ID NO: 120 |
| Seg2 capture confirm FOR | atcagtgtcattgcccagcg | SEQ ID NO: 121 |
| BOL3 check REV | tctcatcaagcagcaccagc | SEQ ID NO: 122 |
| Seg3 capture confirm REV | tggtgagggcgttaatgtgg | SEQ ID NO: 123 |
| Seg3 capture confirm FOR | ggactgatgatgttgaagccc | SEQ ID NO: 124 |
| YAL037W check REV | tgctgctgtagttggtgagc | SEQ ID NO: 125 |
| Seg4 capture confirm REV | tgccgtgcctatttcgtctgg | SEQ ID NO: 126 |
| Seg4 capture confirm FOR | ggactgcggaactacaagctg | SEQ ID NO: 127 |
| MYO4 check REV | aagcctgctaacaagggcga | SEQ ID NO: 128 |
| Seg5 capture confirm REV | gctttacatcttccaggggtct | SEQ ID NO: 129 |
| Seg5 capture confirm FOR | gagcatgttcgcgttcccat | SEQ ID NO: 130 |
| ATS1 check REV | cgcgcctcaatacggtagag | SEQ ID NO: 131 |
| Seg6 capture confirm REV | ggggaactctctcgcaaggt | SEQ ID NO: 132 |
| Seg6 capture confirm FOR | cgacgggggctagaatcttaga | SEQ ID NO: 133 |
| SYN8 check REV | gtcacagtcacagccacagc | SEQ ID NO: 134 |
| Seg7 capture confirm REV | gctggatagcttgggagcg | SEQ ID NO: 135 |
| Seg7 capture confirm FOR | gccagctgctaagtccattgtc | SEQ ID NO: 136 |
| VPS8 check REV | gtcggcactaggattgtctgc | SEQ ID NO: 137 |
| Seg8 capture confirm REV | caaagttccactgggcaaacc | SEQ ID NO: 138 |
| Seg8 capture confirm FOR | tcccgttaataatgtccataggct | SEQ ID NO: 139 |
| Seg9 capture confirm REV | atagggagcggaaggcacag | SEQ ID NO: 140 |
| Seg9 capture confirm FOR | agcagtacagacaggcagga | SEQ ID NO: 141 |
| BUD14 check FOR | agtctcatcaacatgctcttcct | SEQ ID NO: 142 |
| Seg10 capture confirm REV | acccaggcagtggaggag | SEQ ID NO: 143 |
| Seg 10 capture confirm FOR | cgctcgccatgagatatgct | SEQ ID NO: 144 |
| UIP3 check FOR | tacgcagtcaaacacaccagc | SEQ ID NO: 145 |
| Seg11 capture confirm REV | ctgaaggtgtttgcatggtca | SEQ ID NO: 146 |
| Seg11 capture confirm FOR | ccatggattcaaagccgatacc | SEQ ID NO: 147 |

TABLE 16
Genetic elements present on both sub-telomeric
regions in S. cerevisiae BY ChrI.

| | Feature | | | |
|---|---|---|---|---|
| | Systematic | Feature | Coor- | |
| Feature | Name | Type | dinates | Location |
| YAL069W | YAL069W | ORF | 335- | Left Sub |
| | | | 649 | telomere |
| YAL068W-A | YAL068W-A | ORF | 538- | Left Sub |
| | | | 792 | telomere |
| PAU8 | YAL068C | ORF | 1807- | Left Sub |
| | | | 2169 | telomere |
| YAL067W-A | YAL067W-A | ORF | 2480- | Left Sub |
| | | | 2707 | telomere |
| SEO1 | YAL067C | ORF | 7235- | Left Sub |
| | | | 9016 | telomere |
| YAL066W | YAL066W | ORF | 10091- | Left Sub |
| | | | 10399 | telomere |
| YAL065C | YAL065C | ORF | 11565- | Left Sub |
| | | | 11951 | telomere |
| YAL064W-B | YAL064W-B | ORF | 12046- | Left Sub |
| | | | 12426 | telomere |
| TDA8 | YAL064C-A | ORF | 13363- | Left Sub |
| | | | 13743 | telomere |
| YAL064W | YAL064W | ORF | 21566- | Left Sub |
| | | | 21850 | telomere |
| YALWdelta1 | YALWdelta1 | long | 22230- | Left Sub |
| | | terminal | 22552 | telomere |
| | | repeat | | |
| YAL063C-A | YAL063C-A | ORF | 22395- | Left Sub |
| | | | 22685 | telomere |
| FLO9 | YAL063C | ORF | 24000- | Left Sub |
| | | | 27968 | telomere |
| YAR047C | YAR047C | ORF | 201467- | Right Sub |
| | | | 201787 | telomere |
| FLO1 | YAR050W | ORF | 203403- | Right Sub |
| | | | 208016 | telomere |
| YAR053W | YAR053W | ORF | 208367- | Right Sub |
| | | | 208663 | telomere |
| YARCdelta8 | YARCdelta8 | long | 209448- | Right Sub |
| | | terminal | 209778 | telomere |
| | | repeat | | |
| YAR060C | YAR060C | ORF | 217157- | Right Sub |
| | | | 217492 | telomere |
| YAR061W | YAR061W | pseudogene | 218140- | Right Sub |
| | | | 219145 | telomere |
| YAR064W | YAR064W | ORF | 220198- | Right Sub |
| | | | 220497 | telomere |
| YAR066W | YAR066W | ORF | 221049- | Right Sub |
| | | | 221660 | telomere |
| YAR068W | YAR068W | ORF | 222406- | Right Sub |
| | | | 222891 | telomere |
| YAR069C | YAR069C | ORF | 224011- | Right Sub |
| | | | 224304 | telomere |
| YAR070C | YAR070C | ORF | 224563- | Right Sub |
| | | | 224862 | telomere |
| PHO11 | YAR071W | ORF | 225460- | Right Sub |
| | | | 226863 | telomere |
| IMD1 | YAR073W | ORF | 227742- | Right Sub |
| | | | 228953 | telomere |
| YAR075W | YAR075W | ORF | 228844- | Right Sub |
| | | | 229317 | telomere |

Both sub-telomeres (left and right) and consequently their genetic elements were removed from all the synthetic ChrI constructs. Column 1 (‘Features’) lists the standard ID of each genetic element present on sub-telomeres. Column 2 (‘Systematic feature’) lists the systematic feature of each genetic element present on sub-telomeres. Column 3 (‘Feature type’) associates the type of feature to each genetic element. Column 4 (‘Coordinates’) lists the start and end position of each element based on ChrI sequence coordinates. Column 5 (‘Location’) lists if a given feature is located on the left or right sub-telomere on ChrI.

TABLE 17
Phenotypic assay of strains generated during ChrI
multiple deletion assay and their controls.

| Strain | Doubling Time at 30° C. (min) |
|---|---|
| BY4742 | 88.72931969 |
| BY4742 | 83.39266681 |
| BY4742 | 82.89834573 |
| BY4742 | 83.50118352 |
| BY4742 | 89.13436854 |
| BY4742 | 91.37895703 |
| BY4742 | 91.16826909 |
| BY4742 | 91.6830047 |
| BY syn8Δ | 87.98780256 |
| BY syn8Δ | 81.92212109 |
| BY syn8Δ | 87.85825647 |
| BY syn8Δ | 85.44066121 |
| BY syn8Δ | 92.91753156 |
| BY syn8Δ | 90.06264858 |
| BY syn8Δ | 88.07455688 |
| BY syn8Δ | 86.94347991 |
| BY syn8Δ | 87.77750448 |
| BY synthetic ChrI | 88.24396232 |
| BY synthetic ChrI | 93.65803672 |
| BY synthetic ChrI | 95.50624222 |
| BY synthetic ChrI | 97.04904224 |
| BY synthetic ChrI | 94.27469593 |
| BY synthetic ChrI | 89.61415658 |
| BY synthetic ChrI | 86.72118957 |
| BY synthetic ChrI | 94.01112848 |
| BY synthetic ChrI | 95.84090862 |
| ChrI multiple deletion | 219.094345 |
| ChrI multiple deletion | 214.1159012 |
| ChrI multiple deletion | 225.582891 |
| ChrI multiple deletion | 208.6559921 |
| ChrI multiple deletion | 200.8680417 |
| ChrI multiple deletion | 225.2619949 |
| ChrI multiple deletion | 210.2813049 |
| ChrI multiple deletion | 208.9293032 |
| ChrI multiple deletion | 235.2457741 |
| ChrI multiple deletion | 244.3415846 |
| ChrI multiple deletion | 277.1097999 |
| ChrI multiple deletion | 230.0858969 |
| ChrI multiple deletion syn8Δ | 437.1075259 |
| ChrI multiple deletion syn8Δ | 424.9516265 |
| ChrI multiple deletion syn8Δ | 396.5195141 |
| ChrI multiple deletion syn8Δ | 399.462152 |
| ChrI multiple deletion syn8Δ | 390.0608333 |
| ChrI multiple deletion syn8Δ | 386.8709313 |
| ChrI multiple deletion syn8Δ | 389.6391713 |
| ChrI multiple deletion syn8Δ | 390.8815326 |

Column I (‘Strain’) lists IDs of each strain. The ‘ChrI multiple deletion strain’ contains a version of ChrI where 15 out of the 16 targeted genetic elements were removed in a single yeast transformation. The ‘ChrI multiple deletion strain syn8Δ’ is the former ‘ChrI multiple deletion strain’ whose gene SYN8 was deleted through CRISPR/cas9 cutting and replacement. The ‘BY synthetic ChrI strain’ contains a circular assembled version of ChrI without the sub-telomeres but preserved core region. The ‘BY syn8Δ’ strain is the parental wild-type BY4742 strain whose gene SYN8 was deleted by using CRISPR/CAS9 cutting and replacement. The ‘BY4742 strain’ is the wild type parental S. cerevisiae strain. Column 2 (‘Doubling time at 30° C. (min)’) lists the doubling time of each strain growing on rich media at 30° C.

TABLE 18
Sequences of vectors used for Spar ChrI linearization and SYN8 deletion.

| Vector | Sequence |
|---|---|
| pMM_KANMX | gcgcgtcagcgggtgttggcgggtgtcggggctggcttaactatgcggcatcagagcagattgtactgaga |
| (SEQ ID | gtgcaccatatggacatattgtcgttagaacgcggctacaattaatacataaccttatgtatcatacacatacgat |
| NO: 148) | ttaggtgacactatagaacgccacatttccccgaaaagtgccacctgggtccttttcatcacgtgctataaaaat |
| | aattataatttaaattttttaatataaatatataaattaaaaatagaaagtaaaaaaagaaattaaagaaaaaatagt |
| | ttttgttttccgaagatgtaaaagactctagggggatcgccaacaaatactaccttttatcttgctcttcctgctctc |
| | aggtattaatgccgaattgtttcatcttgtctgtgtagaagaccacacacgaaaatcctgtgattttacattttactt |
| | atcgttaatcgaatgtatatctatttaatctgcttttcttgtctaataaatatatatgtaaagtacgctttttgttgaaatt |
| | ttttaaacctttgtttatttttttttcttcattccgtaactcttctaccttctttatttactttctaaaatccaaatacaaa |
| | acataaaaataaataaacacagagtaaattcccaaattattccatcattaaaagatacgaggcgcgtgtaagttaca |
| | ggcaagcgatccgtcctaagaaaccattattatcatgacattaacctataaaaataggcgtatcacgaggccc |
| | gcctctaccttgcagacccatataatataataactaaataagtaaataagacacacgcgagaacatatatacac |
| | aattacagtaacaataacaagaggacagatactaccaaaatgtgtggggaagcgggtaagctgccacagca |
| | attaatgcacaacatttaacctacattcttccttatcggatcctcaaaacccttaaaaacatatgcctcaccctaac |
| | atattttccaattaaccctcaatatttctctgtcacccggcctctattttccattttcttctttacccgccacgcgtttttt |
| | tctttcaaatttttttcttccttcttctttttcttccacgtcctcttgcataaataaataaaccgttttgaaaccaaactcg |
| | cctctctctctcctttttgaaatatttttgggtttgtttgatcctttccttcccaatctctcttgtttaatatatattcatt |
| | tatatcacgctctctttttatcttcctttttttcctctctcttgtattcttccttcccctttctactcaaaccaagaagaaa |
| | aagaaaaggtcaatctttgttaaagaataggatcttctactacatcagcttttagatttttcacgcttactgcttttttctt |
| | cccaagatcgaaaatttactgaattaacagggccccccctcgaggtcgacggtatcgataagcttgaagcaagc |
| | ctcctgaaagatgggtacccgcccatatgcttgccctgtcgagtcctgcgatcgccgcttttctcgccacgcca |
| | atcttacccgccatatccgcatccataccggtcagaagcccttccagtgtcgaatctgcatgcgtaacttcagtc |
| | gtaatgcgaaccttgtgcgccacatccgcacccacacaggatcccaaaagccgttccaatgtcggatctgtat |
| | gcggaactttagtcgaaaggccgacctgaggcgtcacattcgcacgcacaccggcgagaagccttttgcct |
| | gtgacatttgtgggaggaagtttgccaggaagggcgacctcaagaggcataccaaaatccatacaggtggc |
| | ggaggcacacctgcagctgcgtcgactctagaggatccatctgctggagacatgagagctgccaacctttgg |
| | ccaagcccgctcatgatcaaacgctctaagaagaacagcctggccttgtccctgacggccgaccagatggt |
| | cagtgccttgttggatgctgagccccccatactctattccgagtatgatcctaccagacccttcagtgaagcttc |
| | gatgatgggcttactgaccaacctggcagacagggagctggttcacatgatcaactgggcgaagagggtgc |
| | caggctttgtggatttgaccctccatgatcaggtccaccttctagaatgtgcctggctagagatcctgatgattg |
| | gtctcgtctggcgctccatggagcacccagtgaagctactgtttgctcctaacttgctcttggacaggaaccag |
| | ggaaaatgtgtagagggcatggtggagatcttcgacatgctgctggctacatcatctcggttccgcatgatga |
| | atctgcagggagaggagtttgtgtgcctcaaatctattattttgcttaattctggagtgtacacatttctgtccagc |
| | accctgaagtctctggaagagaaggaccatatccaccgagtcctggacaagatcacagacactttgatccac |
| | ctgatggccaaggcaggcctgaccctgcagcagcagcaccagcggctggcccagctcctcctcatcctctc |
| | ccacatcaggcacatgagtaacaaaggcatggagcatctgtacagcatgaagtgcaagaacgtggtgcccc |
| | tctatgacctgctgctggagatgctggacgcccaccgcctacatgcgcccactagccgtggaggggcatcc |
| | gtggaggagacggaccaaagccacttggccactgcgggctctacttcatcggagctccacttagacggcga |
| | ggacgtggcgatggcgcatgccgacgcgctagacgatttcgatctggacatgttgggggacggggattccc |
| | cggggccgggatttaccccccacgactccgccccctacggcgctctggatatggccgacttcgagtttgagc |
| | agatgtttaccgatgcccttggaattgacgagtacggtgggtaggccggccagcgacatggaggcccagaa |
| | taccctccttgacagtcttgacgtgcgcagctcaggggcatgatgtgactgtcgcccgtacatttagcccatac |
| | atccccatgtataatcatttgcatccatacattttgatggccgcacggcgcgaagcaaaaattacggctcctcg |
| | ctgcagacctgcgagcagggaaacgctcccctcacagacgcgttgaattgtccccacgccgcgcccctgta |
| | gagaaatataaaaggttaggatttgccactgaggttcttctttcatatacttccttttaaaatcttgctaggatacag |
| | ttctcacatcacatccgaacataaacaaccatgggtaaggaaaagactcacgtttcgaggccgcgattaaatt |
| | ccaacatggatgctgatttatatgggtataaatgggctcgcgataatgtcgggcaatcaggtgcgacaatctat |
| | cgattgtatgggaagcccgatgcgccagagttgtttctgaaacatggcaaaggtagcgttgccaatgatgtta |
| | cagatgagatggtcagactaaactggctgacggaatttatgcctcttccgaccatcaagcattttatccgtactc |
| | ctgatgatgcatggttactcaccactgcgatccccggcaaaacagcattccaggtattagaagaatatcctgat |
| | tcaggtgaaaatattgttgatgcgctggcagtgttcctgcgccggttgcattcgattcctgtttgtaattgtcctttt |
| | aacagcgatcgcgtatttcgtctcgctcaggcgcaatcacgaatgaataacggtttggttgatgcgagtgatttt |
| | gatgacgagcgtaatggctggcctgttgaacaagtctggaaagaaatgcataagcttttgccattctcaccgg |
| | attcagtcgtcactcatggtgatttctcacttgataaccttatttttgacgaggggaaattaataggttgtattgatgt |
| | tggacgagtcggaatcgcagaccgataccaggatcttgccatcctatggaactgcctcggtgagttttctcctt |
| | cattacagaaacggctttttcaaaaatatggtattgataatcctgatatgaataaattgcagtttcatttgatgctcg |
| | atgagtttttctaatcagtactgacaataaaaagattcttgttttcaagaacttgtcatttgtatagtttttttatattgta |
| | gttgttctattttaatcaaatgttagcgtgatttatattttttttcgcctcgacatcatctgcccagatgcgaagttaag |
| | tgcgcagaaagtaatatcatgcgtcaatcgtatgtgaatgctggtcgctatactgctgtcgattcgatactaacg |
| | ccgccatccagtgtcgaaaacgagaacctgcattaatgaatcggccaacgcgcggggagaggcggtttgc |
| | gtattgggcgctcttccgcttcctcgctcactgactcgctgcgctcggtcgttcggctgcggcgagcggtatca |
| | gctcactcaaaggcggtaatacggttatccacagaatcaggggataacgcaggaaagaacatgtgagcaaa |
| | aggccagcaaaaggccaggaaccgtaaaaaggccgcgttgctggcgtttttccataggctccgcccccctg |
| | acgagcatcacaaaaatcgacgctcaagtcagaggtggcgaaacccgacaggactataaagataccaggc |
| | gtttccccctggaagctccctcgtgcgctctcctgttccgaccctgccgcttaccggatacctgtccgcctttct |
| | cccttcgggaagcgtggcgctttctcaatgctcacgctgtaggtatctcagttcggtgtaggtcgttcgctccaa |
| | gctgggctgtgtgcacgaaccccccgttcagcccgaccgctgcgccttatccggtaactatcgtcttgagtcc |
| | aacccggtaagacacgacttatcgccactggcagcagccactggtaacaggattagcagagcgaggtatgt |
| | aggcggtgctacagagttcttgaagtggtggcctaactacggctacactagaaggacagtatttggtatctgc |
| | gctctgctgaagccagttaccttcggaaaaagagttggtagctcttgatccggcaaacaaaccaccgctggta |
| | gcggtggtttttttgtttgcaagcagcagattacgcgcagaaaaaaaggatctcaagaagatcctttgatcttttc |
| | tacggggtctgacgctcagtggaacgaaaactcacgttaagggattttggtcatgagattatcaaaaaggatct |
| | tcacctagatccttttaaattaaaaatgaagttttaaatcaatctaaagtatatatgagtaaacttggtctgacagtt |
| | accaatgcttaatcagtgaggcacctatctcagcgatctgtctatttcgttcatccatagttgcctgactccccgt |
| | cgtgtagataactacgatacgggagggcttaccatctggccccagtgctgcaatgataccgcgagacccac |
| | gctcaccggctccagatttatcagcaataaaccagccagccggaagggccgagcgcagaagtggtcctgc |
| | aactttatccgcctccatccagtctattaattgttgccgggaagctagagtaagtagttcgccagttaatagtttg |
| | cgcaacgttgttgccattgctacaggcatcgtggtgtcacgctcgtcgtttggtatggcttcattcagctccggtt |
| | cccaacgatcaaggcgagttacatgatcccccatgttgtgcaaaaaagcggttagctccttcggtcctccgat |
| | cgttgtcagaagtaagttggccgcagtgttatcactcatggttatggcagcactgcataattctcttactgtcatg |
| | ccatccgtaagatgcttttctgtgactggtgagtactcaaccaagtcattctgagaatagtgtatgcggcgaccg |
| | agttgctcttgcccggcgtcaatacgggataataccgcgccacatagcagaactttaaaagtgctcatcattgg |
| | aaaacgttcttcggggcgaaaactctcaaggatcttaccgctgttgagatccagttcgatgtaacccactcgtg |
| | cacccaactgatcttcagcatcttttactttcaccagcgtttctgggtgagcaaaaacaggaaggcaaaatgcc |
| | gcaaaaaagggaataagggcgacacggaaatgttgaatactcatactcttcctttttcaatattattgaagcattt |
| | atcagggttattgtctcatgagcggatacatatttgaatgtatttagaaaaataaacaaataggggttccgcgca |
| | catttccccgaaaagtgccacctgacgtctaagaaaccattattatcatgacattaacctataaaaataggcgta |
| | tcacgaggccctttcgtctcgcgcgtttcggtgatgacggtgaaaacctctgacacatgcagctcccggagac |
| | ggtcacagcttgtctgtaagcggatgccgggagcagacaagcccgtcagg |
| pML104_ | aaaagtgccacctgaacgaagcatctgtgcttcattttgtagaacaaaaatgcaacgcgagagcgctaattttt |
| HIS3 | caaacaaagaatctgagctgcatttttacagaacagaaatgcaacgcgaaagcgctattttaccaacgaagaa |
| (SEQ ID | tctgtgcttcatttttgtaaaacaaaaatgcaacgcgagagcgctaatttttcaaacaaagaatctgagctgcatt |
| NO: 149) | tttacagaacagaaatgcaacgcgagagcgctattttaccaacaaagaatctatacttcttttttgttctacaaaa |
| | atgcatcccgagagcgctatttttctaacaaagcatcttagattactttttttctcctttgtgcgctctataatgcagt |
| | ctcttgataactttttgcactgtaggtccgttaaggttagaagaaggctactttggtgtctattttctcttccataaaa |
| | aaagcctgactccacttcccgcgtttactgattactagcgaagctgcgggtgcattttttcaagataaaggcatc |
| | cccgattatattctataccgatgtggattgcgcatactttgtgaacagaaagtgatagcgttgatgattcttcattg |
| | gtcagaaaattatgaacggtttcttctattttgtctctatatactacgtataggaaatgtttacattttcgtattgttttc |
| | gattcactctatgaatagttcttactacaatttttttgtctaaagagtaatactagagataaacataaaaaatgtaga |
| | ggtcgagtttagatgcaagttcaaggagcgaaaggtggatgggtaggttatatagggatatagcacagagat |
| | atatagcaaagagatacttttgagcaatgtttgtggaagcggtattcgcaatattttagtagctcgttacagtccg |
| | gtgcgtttttggttttttgaaagtgcgtcttcagagcgcttttggttttcaaaagcgctctgaagttcctatactttcta |
| | gagaataggaacttcggaataggaacttcaaagcgtttccgaaaacgagcgcttccgaaaatgcaacgcga |
| | gctgcgcacatacagctcactgttcacgtcgcacctatatctgcgtgttgcctgtatatatatatacatgagaag |
| | aacggcatagtgcgtgtttatgcttaaatgcgtacttatatgcgtctatttatgtaggatgaaaggtagtctagtac |
| | ctcctgtgatattatcccattccatgcggggtatcgtatgcttccttcagcactaccctttagctgttctatatgctg |
| | ccactcctcaattggattagtctcatccttcaatgctatcatttcctttgatattggatcatactaagaaaccattatt |
| | atcatgacattaacctataaaaataggcgtatcacgaggccctttcgtctcgcgcgtttcggtgatgacggtga |
| | aaacctctgacacatgcagctcccggagacggtcacagcttgtctgtaagcggatgccgggagcagacaag |
| | cccgtcagggcgcgtcagcgggtgttggcgggtgtcggggctggcttaactatgcggcatcagagcagatt |
| | gtactgagagtgcaccataaattcccgttttaagagcttggtgagcgctaggagtcactgccaggtatcgtttg |
| | aacacggcattagtcagggaagtcataacacagtcctttcccgcaattttctttttctattactcttggcctcctcta |
| | gtacactctatatttttttatgcctcggtaatgattttcatttttttttttcccctagcggatgactctttttttttcttag |
| | cgattggcattatcacataatgaattatacattatataaagtaatgtgatttcttcgaagaatatactaaaaaatgagca |
| | ggcaagataaacgaaggcaaagatgacagagcagaaagccctagtaaagcgtattacaaatgaaaccaag |
| | attcagattgcgatctctttaaagggtggtcccctagcgatagagcactcgatcttcccagaaaaagaggcag |
| | aagcagtagcagaacaggccacacaatcgcaagtgattaacgtccacacaggtatagggtttctggaccata |
| | tgatacatgctctggccaagcattccggctggtcgctaatcgttgagtgcattggtgacttacacatagacgac |
| | catcacaccactgaagactgcgggattgctctcggtcaagcttttaaagaggccctactggcgcgtggagtaa |
| | aaaggtttggatcaggatttgcgcctttggatgaggcactttccagagcggtggtagatctttcgaacaggccg |
| | tacgcagttgtcgaacttggtttgcaaagggagaaagtaggagatctctcttgcgagatgatcccgcattttctt |
| | gaaagctttgcagaggctagcagaattaccctccacgttgattgtctgcgaggcaagaatgatcatcaccgta |
| | gtgagagtgcgttcaaggctcttgcggttgccataagagaagccacctcgcccaatggtaccaacgatgttcc |
| | ctccaccaaaggtgttcttatgtagtgacaccgattatttaaagctgcagcatacgatatatatacatgtgtatata |
| | tgtatacctatgaatgtcagtaagtatgtatacgaacagtatgatactgaagatgacaaggtaatgcatcattcta |
| | tacgtgtcattctgaacgaggcgcgctttcctttaccgcatcaggaaattgtaagcgttaatattttgttaaaattc |
| | gcgttaaatttttgttaaatcagctcattttttaaccaataggccgaaatcggcaaaatcccttataaatcaaaaga |
| | atagaccgagatagggttgagtgttgttccagtttggaacaagagtccactattaaagaacgtggactccaac |
| | gtcaaagggcgaaaaaccgtctatcagggcgatggcccactacgtgaaccatcaccctaatcaagttttttgg |
| | ggtcgaggtgccgtaaagcactaaatcggaaccctaaagggagcccccgatttagagcttgacggggaaa |
| | gccggcgaacgtggcgagaaaggaagggaagaaagcgaaaggagcgggcgctagggcgctggcaagt |
| | gtagcggtcacgctgcgcgtaaccaccacacccgccgcgcttaatgcgccgctacagggcgcgtccattcg |
| | ccattcaggctgcgcaactgttgggaagggcgatcggtgcgggcctcttcgctattacgccagctggcgaaa |
| | gggggatgtgctgcaaggcgattaagttgggtaacgccagggttttcccagtcacgacgttgtaaaacgacg |
| | gccagtgagcgcgcgtaatacgactcactatagggcgaattgggtaccgggccccccctcgaggtcgacg |
| | gtatcgataagcttgatatcgaattcagtctactagtcagttcgagtttatcattatcaatactgccatttcaaagaa |
| | tacgtaaataattaatagtagtgattttcctaactttatttagtcaaaaaattagccttttaattctgctgtaacccgta |
| | catgcccaaaataggggggggttacacagaatatataacatcgtaggtgtctgggtgaacagtttattcctgg |
| | catccactaaatataatggagcccgctttttaagctggcatccagaaaaaaaaagaatcccagcaccaaaata |
| | ttgttttcttcaccaaccatcagttcataggtccattctcttagcgcaactacagagaacaggggcacaaacag |
| | gcaaaaaacgggcacaacctcaatggagtgatgcaacctgcctggagtaaatgatgacacaaggcaattga |
| | cccacgcatgtatctatctcattttcttacaccttctattaccttctgctctctctgatttggaaaaagctgaaaaaaa |
| | aggttgaaaccagttccctgaaattattcccctacttgactaataagtatataaagacggtaggtattgattgtaat |
| | tctgtaaatctatttcttaaacttcttaaattctacttttatagttagtcttttttttagttttaaaacaccaagaacttag |
| | tttcgaataaacacacataaacaaacaaaagatctgatatggatcgaattagatctcgccaccatggacaagaa |
| | gtattctatcggactggacatcgggactaatagcgtcgggtgggccgtcatcactgacgagtacaaggtgcc |
| | ctctaagaagttcaaggtgctcgggaacaccgaccggcattccatcaagaaaaatctgatcggagctctcctc |
| | tttgattcaggggagaccgctgaagcaacccgcctcaagcggactgctagacggcggtacaccaggagga |
| | agaaccggatttgttaccttcaagagatattctccaacgaaatggcaaaggtcgacgacagcttcttccatagg |
| | ctggaagaatcattcctcgtggaagaggataagaagcatgaacggcatcccatcttcggtaatatcgtcgacg |
| | aggtggcctatcacgagaaatacccaaccatctaccatcttcgcaaaaagctggtggactcaaccgacaagg |
| | cagacctccggcttatctacctggccctggcccacatgattaagttcagaggccacttcctgatcgagggcga |
| | cctcaatcctgacaatagcgatgtggataaactgttcatccagctggtgcagacttacaaccagctctttgaag |
| | agaaccccatcaatgcaagcggagtcgatgccaaggccattctgtcagcccggctgtcaaagagccgcag |
| | acttgagaatcttatcgctcagctgccgggtgaaaagaaaaatggactgttcgggaacctgattgctctttcact |
| | tgggctgactcccaatttcaagtctaatttcgacctggcagaggatgccaagctgcaactgtccaaggacacc |
| | tatgatgacgatctcgacaacctcctggcccagatcggtgaccaatacgccgaccttttccttgctgctaagaa |
| | tctttctgacgccatcctgctgtctgacattctccgcgtgaacactgaaatcaccaaggcccctctttcagcttca |
| | atgattaagcggtatgatgagcaccaccaggacctgaccctgcttaaggcactcgtccggcagcagcttccg |
| | gagaagtacaaggaaatcttctttgaccagtcaaagaatggatacgccggctacatcgacggaggtgcctcc |
| | caagaggaattttataagtttatcaaacctatccttgagaagatggacggcaccgaagagctcctcgtgaaact |
| | gaatcgggaggatctgctgcggaagcagcgcactttcgacaatgggagcattccccaccagatccatcttgg |
| | ggagcttcacgccatccttcggcgccaagaggacttctacccctttcttaaggacaacagggagaagattga |
| | gaaaattctcactttccgcatcccctactacgtgggacccctcgccagaggaaatagccggtttgcttggatga |
| | ccagaaagtcagaagaaactatcactccctggaacttcgaagaggtggtggacaagggagccagcgctca |
| | gtcattcatcgaacggatgactaacttcgataagaacctccccaatgagaaggtcctgccgaaacattccctg |
| | ctctacgagtactttaccgtgtacaacgagctgaccaaggtgaaatatgtcaccgaagggatgaggaagccc |
| | gcattcctgtcaggcgaacaaaagaaggcaattgtggaccttctgttcaagaccaatagaaaggtgaccgtg |
| | aagcagctgaaggaggactatttcaagaaaattgaatgcttcgactctgtggagattagcggggtcgaagatc |
| | ggttcaacgcaagcctgggtacctaccatgatctgcttaagatcatcaaggacaaggattttctggacaatgag |
| | gagaaagaggacatccttgaggacattgtcctgactctcactctgttcgaggaccgggaaatgatcgaggag |
| | aggcttaagacctacgcccatctgttcgacgataaagtgatgaagcaacttaaacggagaagatataccggat |
| | ggggacgccttagccgcaaactcatcaacggaatccgggacaaacagagcggaaagaccattcttgatttc |
| | cttaagagcgacggattcgctaatcgcaacttcatgcaacttatccatgatgattccctgacctttaaggaggac |
| | atccagaaggcccaagtgtctggacaaggtgactcactgcacgagcatatcgcaaatctggctggttcaccc |
| | gctattaagaagggtattctccagaccgtgaaagtcgtggacgagctggtcaaggtgatgggtcgccataaa |
| | ccagagaacattgtcatcgagatggccagggaaaaccagactacccagaagggacagaagaacagcagg |
| | gagcggatgaaaagaattgaggaagggattaaggagctcgggtcacagatccttaaagagcacccggtgg |
| | aaaacacccagcttcagaatgagaagctctatctgtactaccttcaaaatggacgcgatatgtatgtggaccaa |
| | gagcttgatatcaacaggctctcagactacgacgtggaccacatcgtccctcagagcttcctcaaagacgact |
| | caattgacaataaggtgctgactcgctcagacaagaaccggggaaagtcagataacgtgccctcagaggaa |
| | gtcgtgaaaaagatgaagaactattggcgccagcttctgaacgcaaagctaatcactcagcggaagttcgac |
| | aatctcactaaggctgagaggggcggactgagcgaactggacaaagcaggattcattaaacggcaacttgt |
| | ggagactcggcagattactaaacatgtagcccaaatccttgactcacgcatgaataccaagtacgacgaaaa |
| | cgacaaacttatccgcgaggtgaaggtgattaccctgaagtccaagctggtcagcgatttcagaaaggacttt |
| | caattctacaaagtgcgggagatcaataactatcatcatgctcatgacgcatatctgaatgccgtggtgggaac |
| | cgccctaatcaagaagtacccaaagctggaaagcgagttcgtgtacggagactacaaggtctacgacgtgc |
| | gcaagatgattgccaaatctgagcaggagatcggaaaggccaccgcaaagtacttcttctacagcaacatca |
| | tgaatttcttcaagaccgaaatcacccttgcaaacggtgagatccggaagaggccgctcatcgagactaatg |
| | gggagactggcgaaatcgtgtgggacaagggcagagatttcgctaccgtgcgcaaagtgctttctatgcctc |
| | aagtgaacatcgtgaagaaaaccgaggtgcaaaccggaggcttttctaaggaatcaatcctccccaagcgc |
| | aactccgacaagctcattgcaaggaagaaggattgggaccctaagaagtacggcggattcgattcaccaact |
| | gtggcttattctgtcctggtcgtggctaaggtggaaaaaggaaagtctaagaagctcaagagcgtgaaggaa |
| | ctgctgggtatcaccattatggagcgcagctccttcgagaagaacccaattgactttctcgaagccaaaggtta |
| | caaggaagtcaagaaggaccttatcatcaagctcccaaagtatagcctgttcgaactggagaatgggcggaa |
| | gcggatgctcgcctccgctggcgaacttcagaagggtaatgagctggctctcccctccaagtacgtgaatttc |
| | ctctaccttgcaagccattacgagaagctgaaggggagccccgaggacaacgagcaaaagcaactgtttgt |
| | ggagcagcataagcattatctggacgagatcattgagcagatttccgagttttctaaacgcgtcattctcgctga |
| | tgccaacctcgataaagtccttagcgcatacaataagcacagagacaaaccaattcgggagcaggctgaga |
| | atatcatccacctgttcaccctcaccaatcttggtgcccctgccgcattcaagtacttcgacaccaccatcgacc |
| | ggaaacgctatacctccaccaaagaagtgctggacgccaccctcatccaccagagcatcaccggactttac |
| | gaaactcggattgacctctcacagctcggaggggatgagggagctcccaagaaaaagcgcaaggtaggta |
| | gttccggatcctagactctcgaggcgaatttcttatgatttatgatttttattattaaataagttataaaaaaaataag |
| | tgtatacaaattttaaagtgactcttaggttttaaaacgaaaattcttattcttgagtaactctttcctgtaggtcagg |
| | ttgctttctcaggtatagcatgaggtcgctcttattgaccacacctctaccggcatgccgagcaaatgcctgcaa |
| | atcgctccccatttctctagagcggccgtggtatcgtccagtcgggaaacctgtcgtgccagctgcattaatga |
| | atcggccaacgcgcggggagaggcggtttgcgtattgggcgctcttccgcttcctcgctcactgactcgctg |
| | cgctcggtcgttcggctgcggcgagcggtatcagctcactcaaaggcggtaatacggttatccacagaatca |
| | ggggataacgcaggaaagaacatgtgagcaaaaggccagcaaaaggccaggaaccgtaaaaaggccgc |
| | gttgctggcgtttttccataggctccgcccccctgacgagcatcacaaaaatcgacgctcaagtcagaggtgg |
| | cgaaacccgacaggactataaagataccaggcgtttccccctggaagctccctcgtgcgctctcctgttccga |
| | ccctgccgcttaccggatacctgtccgcctttctcccttcgggaagcgtggcgctttctcatagctcacgctgta |
| | ggtatctcagttcggtgtaggtcgttcgctccaagctgggctgtgtgcacgaaccccccgttcagcccgaccg |
| | ctgcgccttatccggtaactatcgtcttgagtccaacccggtaagacacgacttatcgccactggcagcagcc |
| | actggtaacaggattagcagagcgaggtatgtaggcggtgctacagagttcttgaagtggtggcctaactac |
| | ggctacactagaagaacagtatttggtatctgcgctctgctgaagccagttaccttcggaaaaagagttggtag |
| | ctcttgatccggcaaacaaaccaccgctggtagcggtggtttttttgtttgcaagcagcagattacgcgcagaa |
| | aaaaaggatctcaagaagatcctttgatcttttctacggggtctgacgctcagtggaacgaaaactcacgttaa |
| | gggattttggtcatgagattatcaaaaaggatcttcacctagatccttttaaattaaaaatgaagttttaaatcaatc |
| | taaagtatatatgagtaaacttggtctgacagttaccaatgcttaatcagtgaggcacctatctcagcgatctgtc |
| | tatttcgttcatccatagttgcctgactccccgtcgtgtagataactacgatacgggagggcttaccatctggcc |
| | ccagtgctgcaatgataccgcgagacccacgctcaccggctccagatttatcagcaataaaccagccagcc |
| | ggaagggccgagcgcagaagtggtcctgcaactttatccgcctccatccagtctattaattgttgccgggaag |
| | ctagagtaagtagttcgccagttaatagtttgcgcaacgttgttgccattgctacaggcatcgtggtgtcacgct |
| | cgtcgtttggtatggcttcattcagctccggttcccaacgatcaaggcgagttacatgatcccccatgttgtgca |
| | aaaaagcggttagctccttcggtcctccgatcgttgtcagaagtaagttggccgcagtgttatcactcatggtta |
| | tggcagcactgcataattctcttactgtcatgccatccgtaagatgcttttctgtgactggtgagtactcaaccaa |
| | gtcattctgagaatagtgtatgcggcgaccgagttgctcttgcccggcgtcaatacgggataataccgcgcca |
| | catagcagaactttaaaagtgctcatcattggaaaacgttcttcggggcgaaaactctcaaggatcttaccgct |
| | gttgagatccagttcgatgtaacccactcgtgcacccaactgatcttcagcatcttttactttcaccagcgtttctg |
| | ggtgagcaaaaacaggaaggcaaaatgccgcaaaaaagggaataagggcgacacggaaatgttgaatac |
| | tcatactcttcctttttcaatattattgaagcatttatcagggttattgtctcatgagcggatacatatttgaatgtattt |
| | agaaaaataaacaaataggggttccgcgcacatttccccg |
| pRS316 | tcgcgcgtttcggtgatgacggtgaaaacctctgacacatgcagctcccggagacggtcacagcttgtctgta |
| (SEQ ID | agcggatgccgggagcagacaagcccgtcagggcgcgtcagcgggtgttggcgggtgtcggggctggct |
| NO: 150) | taactatgcggcatcagagcagattgtactgagagtgcaccacgcttttcaattcaattcatcattttttttttattctt |
| | ttttttgatttcggtttctttgaaatttttttgattcggtaatctccgaacagaaggaagaacgaaggaaggagcac |
| | agacttagattggtatatatacgcatatgtagtgttgaagaaacatgaaattgcccagtattcttaacccaactgc |
| | acagaacaaaaacctgcaggaaacgaagataaatcatgtcgaaagctacatataaggaacgtgctgctactc |
| | atcctagtcctgttgctgccaagctatttaatatcatgcacgaaaagcaaacaaacttgtgtgcttcattggatgtt |
| | cgtaccaccaaggaattactggagttagttgaagcattaggtcccaaaatttgtttactaaaaacacatgtggat |
| | atcttgactgatttttccatggagggcacagttaagccgctaaaggcattatccgccaagtacaattttttactctt |
| | cgaagacagaaaatttgctgacattggtaatacagtcaaattgcagtactctgcgggtgtatacagaatagcag |
| | aatgggcagacattacgaatgcacacggtgtggtgggcccaggtattgttagcggtttgaagcaggcggca |
| | gaagaagtaacaaaggaacctagaggccttttgatgttagcagaattgtcatgcaagggctccctatctactg |
| | gagaatatactaagggtactgttgacattgcgaagagcgacaaagattttgttatcggctttattgctcaaagag |
| | acatgggtggaagagatgaaggttacgattggttgattatgacacccggtgtgggtttagatgacaagggag |
| | acgcattgggtcaacagtatagaaccgtggatgatgtggtctctacaggatctgacattattattgttggaagag |
| | gactatttgcaaagggaagggatgctaaggtagagggtgaacgttacagaaaagcaggctgggaagcatat |
| | ttgagaagatgcggccagcaaaactaaaaaactgtattataagtaaatgcatgtatactaaactcacaaattag |
| | agcttcaatttaattatatcagttattaccctgcggtgtgaaataccgcacagatgcgtaaggagaaaataccgc |
| | atcaggaaattgtaaacgttaatattttgttaaaattcgcgttaaatttttgttaaatcagctcattttttaaccaatag |
| | gccgaaatcggcaaaatcccttataaatcaaaagaatagaccgagatagggttgagtgttgttccagtttggaa |
| | caagagtccactattaaagaacgtggactccaacgtcaaagggcgaaaaaccgtctatcagggcgatggcc |
| | cactacgtgaaccatcaccctaatcaagttttttggggtcgaggtgccgtaaagcactaaatcggaaccctaaa |
| | gggagcccccgatttagagcttgacggggaaagccggcgaacgtggcgagaaaggaagggaagaaagc |
| | gaaaggagcgggcgctagggcgctggcaagtgtagcggtcacgctgcgcgtaaccaccacacccgccgc |
| | gcttaatgcgccgctacagggcgcgtcgcgccattcgccattcaggctgcgcaactgttgggaagggcgat |
| | cggtgcgggcctcttcgctattacgccagctggcgaaggggggatgtgctgcaaggcgattaagttgggtaa |
| | cgccagggttttcccagtcacgacgttgtaaaacgacggccagtgaattgtaatacgactcactatagggcga |
| | attggagctccaccgcggtggcggccgctctagaactagtggatcccccgggctgcaggaattcgatatcaa |
| | gcttatcgataccgtcgacctcgagggggggcccggtacccagcttttgttccctttagtgagggttaattccg |
| | agcttggcgtaatcatggtcatagctgtttcctgtgtgaaattgttatccgctcacaattccacacaacataggag |
| | ccggaagcataaagtgtaaagcctggggtgcctaatgagtgaggtaactcacattaattgcgttgcgctcact |
| | gcccgctttccagtcgggaaacctgtcgtgccagctgcattaatgaatcggccaacgcgcggggagaggcg |
| | gtttgcgtattgggcgctcttccgcttcctcgctcactgactcgctgcgctcggtcgttcggctgcggcgagcg |
| | gtatcagctcactcaaaggcggtaatacggttatccacagaatcaggggataacgcaggaaagaacatgtga |
| | gcaaaaggccagcaaaaggccaggaaccgtaaaaaggccgcgttgctggcgtttttccataggctcggccc |
| | ccctgacgagcatcacaaaaatcgacgctcaagtcagaggtggcgaaacccgacaggactataaagatac |
| | caggcgttcccccctggaagctccctcgtgcgctctcctgttccgaccctgccgcttaccggatacctgtccg |
| | cctttctcccttcgggaagcgtggcgctttctcaatgctcacgctgtaggtatctcagttcggtgtaggtcgttcg |
| | ctccaagctgggctgtgtgcacgaaccccccgttcagcccgaccgctgcgccttatccggtaactatcgtctt |
| | gagtccaacccggtaagacacgacttatcgccactggcagcagccactggtaacaggattagcagagcga |
| | ggtatgtaggcggtgctacagagttcttgaagtggtggcctaactacggctacactagaaggacagtatttggt |
| | atctgcgctctgctgaagccagttaccttcggaaaaagagttggtagctcttgatccggcaaacaaaccaccg |
| | ctggtagcggtggtttttttgtttgcaagcagcagattacgcgcagaaaaaaaggatctcaagaagatcctttg |
| | atcttttctacggggtctgacgctcagtggaacgaaaactcacgttaagggattttggtcatgagattatcaaaa |
| | aggatcttcacctagatccttttaaattaaaaatgaagttttaaatcaatctaaagtatatatgagtaaacttggtct |
| | gacagttaccaatgcttaatcagtgaggcacctatctcagcgatctgtctatttcgttcatccatagttgcctgact |
| | gcccgtcgtgtagataactacgatacgggagggcttaccatctggccccagtgctgcaatgataccgcgaga |
| | cccacgctcaccggctccagatttatcagcaataaaccagccagccggaagggccgagcgcagaagtggt |
| | cctgcaactttatccgcctccatccagtctattaattgttgccgggaagctagagtaagtagttcgccagttaata |
| | gtttgcgcaacgttgttgccattgctacaggcatcgtggtgtcacgctcgtcgtttggtatggcttcattcagctc |
| | cggttcccaacgatcaaggcgagttacatgatcccccatgttgtgaaaaaaagcggttagctccttcggtcctc |
| | cgatcgttgtcagaagtaagttggccgcagtgttatcactcatggttatggcagcactgcataattctcttactgt |
| | catgccatccgtaagatgcttttctgtgactggtgagtactcaaccaagtcattctgagaatagtgtatgcggcg |
| | accgagttgctcttgcccggcgtcaatacgggataataccgcgccacatagcagaactttaaaagtgctcatc |
| | attggaaaacgttcttcggggcgaaaactctcaaggatcttaccgctgttgagatccagttcgatgtaacccac |
| | tcgtgcacccaactgatcttcagcatcttttactttcaccagcgtttctgggtgagcaaaaacaggaaggcaaa |
| | atgccgcaaaaaagggaataagggcgacacggaaatgttgaatactcatactcttcctttttcaatattattgaa |
| | gcatttatcagggttattgtctcatgagcggatacatatttgaatgtatttagaaaaataaacaaataggggttcc |
| | gcgcacatttccccgaaaagtgccacctgggtccttttcatcacgtgctataaaaataattataatttaaatttttta |
| | atataaatatataaattaaaaatagaaagtaaaaaaagaaattaaagaaaaaatagtttttgttttccgaagatgta |
| | aaagactctagggggatcgccaacaaatactaccttttatcttgctcttcctgctctcaggtattaatgccgaatt |
| | gtttcatcttgtctgtgtagaagaccacacacgaaaatcctgtgattttacattttacttatcgttaatcgaatgtata |
| | tctatttaatctgcttttcttgtctaataaatatatatgtaaagtacgctttttgttgaaattttttaaacctttgtttatt |
| | tttttttcttcattccgtaactcttctaccttctttatttactttctaaaatccaaatacaaaacataaaaataaataaacaca |
| | gagtaaattcccaaattattccatcattaaaagatacgaggcgcgtgtaagttacaggcaagcgatccgtccta |
| | agaaaccattattatcatgacattaacctataaaaataggcgtatcacgaggccctttcgtc |
| pRS316_ | tctacggggtctgacgctcagtggaacgaaaactcacgttaagggattttggtcatgagattatcaaaaaggat |
| NATMX | cttcacctagatccttttaaattaaaaatgaagttttaaatcaatctaaagtatatatgagtaaacttggtctgacag |
| (SEQ ID | ttaccaatgcttaatcagtgaggcacctatctcagcgatctgtctatttcgttcatccatagttgcctgactgcccg |
| NO: 151) | tcgtgtagataactacgatacgggagggcttaccatctggccccagtgctgcaatgataccgcgagacccac |
| | gctcaccggctccagatttatcagcaataaaccagccagccggaagggccgagcgcagaagtggtcctgc |
| | aactttatccgcctccatccagtctattaattgttgccgggaagctagagtaagtagttcgccagttaatagtttg |
| | cgcaacgttgttgccattgctacaggcatcgtggtgtcacgctcgtcgtttggtatggcttcattcagctccggtt |
| | cccaacgatcaaggcgagttacatgatcccccatgttgtgaaaaaaagcggttagctccttcggtcctccgat |
| | cgttgtcagaagtaagttggccgcagtgttatcactcatggttatggcagcactgcataattctcttactgtcatg |
| | ccatccgtaagatgcttttctgtgactggtgagtactcaaccaagtcattctgagaatagtgtatgcggcgaccg |
| | agttgctcttgcccggcgtcaatacgggataataccgcgccacatagcagaactttaaaagtgctcatcattgg |
| | aaaacgttcttcggggcgaaaactctcaaggatcttaccgctgttgagatccagttcgatgtaacccactcgtg |
| | cacccaactgatcttcagcatcttttactttcaccagcgtttctgggtgagcaaaaacaggaaggcaaaatgcc |
| | gcaaaaaagggaataagggcgacacggaaatgttgaatactcatactcttcctttttcaatattattgaagcattt |
| | atcagggttattgtctcatgagcggatacatatttgaatgtatttagaaaaataaacaaataggggttccgcgca |
| | catttccccgaaaagtgccacctgggtccttttcatcacgtgctataaaaataattataatttaaattttttaatataa |
| | atatataaattaaaaatagaaagtaaaaaaagaaattaaagaaaaaatagtttttgttttccgaagatgtaaaaga |
| | ctctagggggatcgccaacaaatactaccttttatcttgctcttcctgctctcaggtattaatgccgaattgtttcat |
| | cttgtctgtgtagaagaccacacacgaaaatcctgtgattttacattttacttatcgttaatcgaatgtatatctattt |
| | aatctgcttttcttgtctaataaatatatatgtaaagtacgctttttgttgaaattttttaaacctttgtttatttttttt |
| | tcttcattccgtaactcttctaccttctttatttactttctaaaatccaaatacaaaacataaaaataaataaacacagagt |
| | aaattcccaaattattccatcattaaaagatacgaggcgcgtgtaagttacaggcaagcgatccgtcctaagaaa |
| | ccattattatcatgacattaacctataaaaataggcgtatcacgaggccctttcgtctcgcgcgtttcggtgatga |
| | cggtgaaaacctctgacacatgcagctcccggagacggtcacagcttgtctgtaagcggatgccgggagca |
| | gacaagcccgtcagggcgcgtcagcgggtgttggcgggtgtcggggctggcttaactatgcggcatcaga |
| | gcagattgtaagatctgtttagcttgccttgtccccgccgggtcacccggccagcgacatggaggcccagaa |
| | taccctccttgacagtcttgacgtgcgcagctcaggggcatgatgtgactgtcgcccgtacatttagcccatac |
| | atccccatgtataatcatttgcatccatacattttgatggccgcacggcgcgaagcaaaaattacggctcctcg |
| | ctgcagacctgcgagcagggaaacgctcccctcacagacgcgttgaattgtccccacgccgcgcccctgta |
| | gagaaatataaaaggttaggatttgccactgaggttcttctttcatatacttccttttaaaatcttgctaggatacag |
| | ttctcacatcacatccgaacataaacaaccatgggtaaaaagcctgaactcaccgcgacgtctgtcgagaagt |
| | ttctgatcgaaaagttcgacagcgtctccgacctgatgcagctctcggagggcgaagaatctcgtgctttcag |
| | cttcgatgtaggagggcgtggatatgtcctgcgggtaaatagctgcgccgatggtttctacaaagatcgttatg |
| | tttatcggcactttgcatcggccgcgctcccgattccggaagtgcttgacattggggaattcagcgagagcct |
| | gacctattgcatctcccgccgtgcacagggtgtcacgttgcaagacctgcctgaaaccgaactgcccgctgtt |
| | ctgcagccggtcgcggaggccatggatgcgatcgctgcggccgatcttagccagacgagcgggttcggcc |
| | cattcggaccgcaaggaatcggtcaatacactacatggcgtgatttcatatgcgcgattgctgatccccatgtg |
| | tatcactggcaaactgtgatggacgacaccgtcagtgcgtccgtcgcgcaggctctcgatgagctgatgcttt |
| | gggccgaggactgccccgaagtccggcacctcgtgcacgcggatttcggctccaacaatgtcctgacgga |
| | caatggccgcataacagcggtcattgactggagcgaggcgatgttcggggattcccaatacgaggtcgcca |
| | acatcttcttctggaggccgtggttggcttgtatggagcagcagacgcgctacttcgagcggaggcatccgg |
| | agcttgcaggatcgccgcggctccgggcgtatatgctccgcattggtcttgaccaactctatcagagcttggtt |
| | gacggcaatttcgatgatgcagcttgggcgcagggtcgatgcgacgcaatcgtccgatccggagccggga |
| | ctgtcgggcgtacacaaatcgcccgcagaagcgcggccgtctggaccgatggctgtgtagaagtactcgcc |
| | gatagtggaaaccgacgccccagcactcgtccgagggcaaaggaataatcagtactgacaataaaaagatt |
| | cttgttttcaagaacttgtcatttgtatagtttttttatattgtagttgttctattttaatcaaatgttagcgtgatttata |
| | ttttttttcgcctcgacatcatctgcccagatgcgaagttaagtgcgcagaaagtaatatcatgcgtcaatcgtatgt |
| | gaatgctggtcgctatactgctgtcgattcgatactaacgccgccatccagtgtcgaaaacgagctcgaattca |
| | tcgatgtcagttattaccctgcggtgtgaaataccgcacagatgcgtaaggagaaaataccgcatcaggaaat |
| | tgtaaacgttaatattttgttaaaattcgcgttaaatttttgttaaatcagctcattttttaaccaataggccgaaatcg |
| | gcaaaatcccttataaatcaaaagaatagaccgagatagggttgagtgttgttccagtttggaacaagagtcca |
| | ctattaaagaacgtggactccaacgtcaaagggcgaaaaaccgtctatcagggcgatggcccactacgtga |
| | accatcaccctaatcaagttttttggggtcgaggtgccgtaaagcactaaatcggaaccctaaagggagcccc |
| | cgatttagagcttgacggggaaagccggcgaacgtggcgagaaaggaagggaagaaagcgaaaggagc |
| | gggcgctagggcgctggcaagtgtagcggtcacgctgcgcgtaaccaccacacccgccgcgcttaatgcg |
| | ccgctacagggcgcgtcgcgccattcgccattcaggctgcgcaactgttgggaagggcgatcggtgcggg |
| | cctcttcgctattacgccagctggcgaaggggggatgtgctgcaaggcgattaagttgggtaacgccagggt |
| | tttcccagtcacgacgttgtaaaacgacggccagtgaattgtaatacgactcactatagggcgaattggagctc |
| | caccgcggtggcggccgctctagaactagtggatcccccgggctgcaggaattcgatatcaagcttatcgat |
| | accgtcgacctcgagggggggcccggtacccagcttttgttccctttagtgagggttaattccgagcttggcgt |
| | aatcatggtcatagctgtttcctgtgtgaaattgttatccgctcacaattccacacaacataggagccggaagca |
| | taaagtgtaaagcctggggtgcctaatgagtgaggtaactcacattaattgcgttgcgctcactgcccgctttcc |
| | agtcgggaaacctgtcgtgccagctgcattaatgaatcggccaacgcgcggggagaggcggtttgcgtatt |
| | gggcgctcttccgcttcctcgctcactgactcgctgcgctcggtcgttcggctgcggcgagcggtatcagctc |
| | actcaaaggcggtaatacggttatccacagaatcaggggataacgcaggaaagaacatgtgagcaaaagg |
| | ccagcaaaaggccaggaaccgtaaaaaggccgcgttgctggcgtttttccataggctcggcccccctgacg |
| | agcatcacaaaaatcgacgctcaagtcagaggtggcgaaacccgacaggactataaagataccaggcgttc |
| | ccccctggaagctccctcgtgcgctctcctgttccgaccctgccgcttaccggatacctgtccgcctttctccct |
| | tcgggaagcgtggcgctttctcaatgctcacgctgtaggtatctcagttcggtgtaggtcgttcgctccaagct |
| | gggctgtgtgcacgaaccccccgttcagcccgaccgctgcgccttatccggtaactatcgtcttgagtccaac |
| | ccggtaagacacgacttatcgccactggcagcagccactggtaacaggattagcagagcgaggtatgtagg |
| | cggtgctacagagttcttgaagtggtggcctaactacggctacactagaaggacagtatttggtatctgcgctc |
| | tgctgaagccagttaccttcggaaaaagagttggtagctcttgatccggcaaacaaaccaccgctggtagcg |
| | gtggtttttttgtttgcaagcagcagattacgcgcagaaaaaaaggatctcaagaagatcctttgatcttt |

TABLE 19
gRNAs used during S.par ChrI linearization and SYN8 deletion. Column 1
(‘gRNAs’) lists the gRNAs IDs. The ‘up’ and ‘down’ annotations refer to the
side of a segment targeted by a guide, Column 2 (‘sequence’) lists the
target sequence of each gRNA.

| gRNA | Sequence | SEQ ID NO |
|---|---|---|
| SYN8 gRNA1 | ggacaaaattcctgatacgg | SEQ ID NO: 152 |
| SYN8 gRNA2 | ggatgacgatgaacaagacg | SEQ ID NO: 153 |
| pASC2 gRNA up 1 | gcgtagggaacgtcgcaaag | SEQ ID NO: 154 |
| pASC2 gRNA up 2 | ccgcacagatgcgtaaggta | SEQ ID NO: 155 |
| pASC2 gRNA down 1 | ggtgtggaaaagctccctaa | SEQ ID NO: 156 |
| pASC2 gRNA down 2 | tatccctactcactcaaagg | SEQ ID NO: 157 |

### Example 2. Material and Methods

Production of target-specific cloning vectors. To produce the BAC/YAC cloning vector pASC1, we performed a four-piece Gibson assembly (Gibson et al., Nat Methods 6, 343-5 (2009).) with the pCC1BAC CopyControl plasmid from Epicentre Biotechnologies, a portion of the pRS316 plasmid (Sikorski et al., Genetics 122, 19-27 (1989).) (ATCC #77145) that included ARSH4, CEN6, and URA3, and two DNA blocks containing I-SceI sites. To create the necessary homology for Gibson assembly, pCC1BAC and the portion of pRS316 were amplified by PCR with tailed primers. The two DNA blocks containing I-SceI sites were ordered from Twist Bioscience and also contain a multiple cloning site (MCS) that is used as a homology sequence between the two blocks. Correct assembly of all four pieces produced a vector with a multiple cloning site flanked by two I-SceI sites. To prepare this cloning vector for a specific target segment, we add a −500 bp cloning cassette to pASC1 at the MCS. Each cloning cassette contains two −150 bp homology arms flanked by −100 bp adapters and separated by 30 bp of restriction sites that are used for vector linearization. Cloning cassettes were ordered from Twist Bioscience. Addition of a cloning cassette to pASC1 was done by restriction digestion and ligation. Equimolar amounts of pASC1 and a cloning cassette were digested with EcoRI and SphI and ligated using T4 DNA ligase. After addition of a cloning cassette, the vector was transformed into TransforMax EP1300 cells (LGC Biosearch Technologies) and high copy number was induced with Epicentre's CopyControl system via a copy control induction solution (LGC Biosearch Technologies). Large quantities of vectors were then harvested by ZymoPURE II plasmid midiprep kit (Zymo Research).

In vitro transcription of gRNAs. The gRNAs for all CRISPR/Cas9 cutting were produced by in vitro transcription (Kannan et al., Sci Rep 6, 30714 (2016)). For a given gRNA, we generated a dsDNA template by fusing two ssDNA oligonucleotides, one including the tracrRNA and the other the target-specific crRNA, using PCR. After PCR products were purified using the DNA Clean and Concentrator-5 kit, we combined 150 ng of purified PCR with 10 ul of RiboMAX 2× buffer and 2 ul of T7 express enzyme from the T7 RiboMAX Express Large Scale RNA Production System (Promega). Water was used to bring each reaction to a total volume of 20 ul. In vitro transcription reactions were incubated at 37° C. for ≥4 hrs. We then added 2 ul of DNAse and incubated reactions for an additional 18 mins at 37° C. We cleaned gRNAs using the RNA Clean & Concentrator-5 kit (Zymo Research) and stored them at −20° C. until use.

Cloning of natural genomic segments. We clone target segments by co-transforming a linear version of pASC1 that possess homology arms matching the ends of a target segment, a repair template containing kan (KanMX), and gRNAs into a Saccharomyces strain that constitutively expresses Streptococcus pyogenes Cas9 either from a chromosomally-encoded construct or from a plasmid. Prior to transformation, the cloning vector is linearized by cutting between the homology arms using XhoI and AvrII. The repair template is produced through a PCR reaction using a modified pRS316 plasmid in which URA3 was replaced with KanMX as the template. The primers are designed to flank KanMX and contain 40 bp homology tails that match genomic sites adjacent to a target segment. Yeast cells were transformed with 200 ng of linearized vector, 200 ng of repair template, and 1 ug total of a mix of multiple gRNAs. Typically, we included six distinct gRNAs, three targeting each side of a segment. Cells were transferred to 2 mm electroporation cuvettes and electroporated at 2.5 kV, 200Ω, and 25 uF⁴⁰. Transformants were recovered for 2-3 hours in YPDS, a 50:50 mix of YPD (2% glucose, 1% yeast extract, and 2% peptone) and 1M sorbitol, and plated on SC Ura- plates containing G418 to select for the pASC1 vector and use of the repair template, respectively. After two days, transformants were checked by colony PCR at both junctions between pASC1 and a cloned segment.

Amplification of cloned segments. Cloned segments were extracted from yeast using the Zymo Research ZymoPURE II bacterial midiprep kit and the following steps. The yeast strain containing a cloned segment was inoculated into 50 ml of SC lacking uracil and containing G418, and grown for 12-16 hrs at 30° C. on a shaking incubator. Cells were pelleted and washed twice using sterile water. Cells were then converted to spheroplasts by resuspension in 3 ml of lysis buffer (1 M sorbitol, 100 mM EDTA pH 8, and 14 mM β-mercaptoethanol) and 1 ml of lyticase (15 U/ul). This suspension was incubated at 37° C. for 1 hr with shaking and inspected for spheroplasts every 15 mins. After the majority of cells had their cell wall removed (≥75%), the spheroplasts were pelleted and washed with sterile water twice. The remaining steps proceeded according to manufacturer recommendations. To prevent DNA shearing, vortex steps were avoided, and wide bore pipette tips were employed. The plasmid containing the cloned segment was then transformed into EPI300 cells (LGC Biosearch Technologies) and transformants were selected by growing in LB chloramphenicol plates (30 μg/ml). Presence of the correct cloned segment was checked by junction PCRs and confirmed transformants were grown overnight at 37° C. on a shaking incubator. High copy plasmid induction was done by transferring 4.5 ml of overnight growing cells to 45 ml of LB supplemented with chloramphenicol (30 ug/ml) and 50 ul of copy control induction solution (LGC Biosearch Technologies). Cells were grown for 5 hrs and plasmids extracted using the midiprep kit described before, using manufacturer recommendation. The cloned segments were liberated from the cloning vector by digestion with I-SceI, followed by 0.5% agarose gel electrophoresis at 70 V for 90 mins, and purification with the Zymoclean Large Fragment DNA Recovery kit (Zymo Research).

Construction of the centromere cassette. We generated the centromere cassette by adding kan (KanMX) and a loxP site to the pRS316 plasmid right after its CEN/ARS region. The pRS316 vector, the KanMX cassette, and a loxP site were amplified using primers with homology tails. The molecules were mixed in equimolar amounts and ligated using Gibson Assembly Master Mix (New England Biolabs). These assemblies was then transformed into Dh5α cells and the correct assembly was identified by PCR and Sanger sequencing. To generate the centromere cassettes used in synthetic chromosome assemblies, we amplified the centromere cassette region of the plasmid with tailed primers containing appropriate adapters.

Chromosome assembly. Synthetic chromosomes were assembled as circular molecules including segments, a centromere cassette with appropriate adapters, and a modified BAC/YAC vector named pASC2, which lacks CEN/ARS and contains HIS3 instead of URA3. A given assembly was performed by co-transforming >500 ng of each purified segment, 200 ng of the centromere cassette, and 200 ng of linearized pASC2 into BY. Transformation was performed using a standard PEG/LiAc method (Gietz et al., method. Nat. Protoc. 2, 31-34 (2007).), but extra care was taken when handling DNA solutions to avoid DNA shearing. All vortex steps were replaced by gentle manual shaking and pipetting with wide bore tips. Transformants containing assemblies were selected on SC lacking histidine and containing G418. Correct assemblies were identified by junction PCRs and confirmed by Oxford Nanopore sequencing.

Elimination of native chromosomes. We used CRISPR/Cas9 to place pGAL1 114 bp upstream of the centromere on the native ChrI in BY. In the presence of galactose, pGAL1 drives transcription through the centromere, destabilizing its function and resulting in native ChrI loss in some cells (Richardson et al., Science 355, 1040-1044 (2017); Hill et al., Mol. Cell. Biol. 7, 2397-2405 (1987).). We also marked the native ChrI with URA3, enabling selection for native ChrI loss. After assembly, cells possessing an assembly were selected on SC lacking histidine and containing G418, and then replicated onto plates that lacked histidine and contained galactose. Cells were grown for two days and then replicated into SC plates lacking histidine that were supplemented with 5-FOA. Elimination of native ChrI was confirmed by diagnostic PCRs of sites that present on the native ChrI but not a synthetic ChrI.

Linearization of synthetic S. paradoxus ChrI. The BY strain containing the assembled S. paradoxus ChrI was first transformed with a modified version of the pML104 Cas9 plasmid⁴², which had URA3 replaced with HIS3 (Table 18). To linearize the S. paradoxus ChrI, we used electroporation to co-transform two telomere cassettes and two pairs of in vitro transcribed gRNAs that target the ends of the pASC2 vector (Table 19). The left telomere cassette was generated by amplification of URA3 in the pRS316 vector using primers with tails that added a telomere seed sequence and homology to S. paradoxus ChrI (For: TGTGTGGTGTGTGGTGTGTGTGGGTGTGTGGTGTGTGGGT CTGTAAGCGGATGCCGG (SEQ ID NO: 158); Rev: CTCCTTACGCATCTGTGCG TACCCTTTAAAATCTCATTGGCTCGTGATTAATTTGTTCTGTGCTGCTGAATATTCATGC)(SEQ ID NO: 159). The right telomere cassette was generated by amplifying nat (NatMX) from a modified pRS316 plasmid, which had URA3 replaced with nat (NatMX) (For: TTACATATCCTCTACACCGAGCGCGTCGACCCGTCGAATGGTTTAGCTTGCCTTGTC CCC (SEQ ID NO: 160); Rev: GGCGGCGTTAGTATCGAATCCACC CACCACACACACCCACACACACCACACACCCACCCA) (SEQ ID NO: 161). Linearization occurs when the gRNAs create double strand breaks at both ends of pASC2, which are repaired by homologous recombination between S. paradoxus ChrI and the left and right telomere cassettes. Strains with a linear ChrI will have URA3 and nat (NatMX) and will lack the HIS3 present on pASC2. Linearization was confirmed phenotypically and by PCR of the linearization sites.

Deletion of SYN8. We used Cas9 to delete SYN8 from BY and the ChrI multiple deletion strain. Both strains were electroporated with the pML104 Cas9 vector that had URA3 replaced with HIS3. We used a 2 mm cuvette and 2.5 kV, 200Ω, and 25 uF. Transformants were selected on SC His-. We then performed a second electroporation with 200 ng of a nat (NatMX) repair template with homology arms targeting the sites adjacent to the cut and 1 ug total of gRNAs targeting SYN8 (Table 19). We added the homology arms to nat (NatMX) by tailed PCR using the modified pRS316 vector as the template (For: GGGCTATAAAGTATATATAGATACAAATATATGATGAATCGTTTAGCTTGCCTTGTC CCC (SEQ ID NO: 162); Rev: GAATAAAATTTCCCAGCACGACTTTGATCACC CGAAAGGGGGCGGCGTTAGTATCGAATC) (SEQ ID NO: 163). Transformants were recovered for 2-3 hours in YPDS and plated on YPD plates containing 200 ug/ul of nourseothricin (Goldbio, USA).

Genomic DNA extraction. Strains were streaked from a −80° C. freezer stock onto YPD plates containing G418 and allowed to grow for two days at 30° C. A single colony was then used to inoculate a 5 ml overnight culture in YPD containing G418 which was placed in a shaker at 30° C. The 5 ml overnight culture was then inoculated into 50 ml+G418 in a 250 ml Erlenmeyer flask. The cells were shaken overnight at 30° C. Prior to extraction, these cultures were normalized to 7.0×10⁹ number of cells in YPD. Cells were then placed into 50 ml falcon tubes, centrifuged at 3000×g for 10 mins at 4° C., and supernatant was decanted. Cell pellets were gently washed with 10 ml of PBS and centrifuged again at 3000×g for 10 mins at 4° C. Supernatant was then decanted, and a fresh 10 mL of PBS was added. This process was repeated twice. After the wash steps 4 ml of Y1 (1M sorbitol, 100 mM EDTA pH 8.0, and 14 mM beta mercaptoethanol which was added immediately before use) were added to the cells along with 1 ml of lyticase (15 U/ul). The tubes were transferred back to the shaking incubator at 30° C. for ≥1 hr, with hand inversion done every 10 mins to prevent the cells from settling. OD was checked periodically and once the cells had lost 80% of their OD660, spheroplasting was considered complete. Spheroplasts were centrifuged at 5000×g for 10 mins at 4° C., and the supernatant was decanted and replaced with 5 ml of Qiagen G2 buffer with 15 ul of RNase A and 300 ul of Proteinase K. The solution was incubated at 55° C. for ≥2 hrs. A Qiagen 100/G Genomic tip was equalized with 4 mL of QBT buffer. While this was flowing through the samples were centrifuged at 5000×g for 10 mins at 4° C. to spin down cell debris. The sample was passed through the column, collected and repassed through the column a second time. The column was washed with 2×7.5 ml of buffer QC, then eluted with 5 ml of buffer QF. The collected elution was passed through the column a second time, then 3.5 ml of molecular grade isopropanol was added along with 0.01× volumes of filter sterilized sodium acetate. The samples were placed in a −20° C. to precipitate for 48 hrs. After precipitation the samples were spun down at 21000×g for 10 mins at 4° C. to collect the DNA in a Lo-bind tube. The ethanol was decanted, and the ethanol was allowed to evaporate for 1 min. An extraction buffer (EB) of 1,080 ul of 10 mM tris-HCl, 1 mM EDTA Ph 9.0, and 45 ul Triton X-100 0.5% (v/v) was created fresh before each use. 200 ul of EB buffer was added to the DNA. The DNA was then allowed to dissolve at 55° C. for 30 mins followed by 2-3 hours at 30° C.

Oxford Nanopore library preparation. For Oxford Nanopore library preparation, the SQK-LSK109 and EXP-NBD104 protocols were used with the following modifications. At the end of each bead clean up, DNA was eluted in EB at 37° C. for 10 mins to allow for the recovery of larger molecules. The library was then barcoded and multiplexed with up to 10 samples. These multiplexes were sequenced on R9.4.1 Chemistry, MIN-106 flow cells. The .fast5 archive was Tarbelled, gunzipped, and uploaded onto the USC's Center for Advanced Research Computing (CARC) Discovery cluster. Basecalling was performed using Guppy (v. 6.0.1), which was contained in a Singularity container using the configuration dna_r9.4.1_450bps_sup.cfg with 16 threads and a V100 GPU. The .fastq files were demultiplexed and trimmed of barcodes in Guppy using standard parameters.

Structural analysis of synthetic chromosomes. For each strain carrying a synthetic chromosome, we built a reference genome in silico using appropriate data from BY, RM, or S. paradoxus CBS5829. Because all assemblies were performed in BY, Chromosomes II-XIV, as well as the mitochondrial genome and 2-um plasmid, were always taken from the BY reference genome. The only part of the reference genomes that varied was ChrI. Reads were mapped against the appropriate reference using minimap2 (Li et al., Bioinformatics 34, 3094-3100 (2018).) (v. 2.24-r1122, -ax map-ont). Using samtools (Danecek et al., GigaScience 10, giab008 (2021); Bonfield et al., GigaScience 10, giab007 (2021).) (v1.15, htslib v1.15, view -bS), the .sam was converted into a bam file, sorted, and indexed. We then used bamtools (v2.5.2) to split the .bam file by chromosome. The .bam file for ChrI was extracted and used in both Nanocaller and Sniffles with variant calls being made when both programs agreed upon a variant and the .bam files could be visually inspected to verify each call. Reads spanning adapters were extracted using samtools (v1.15, htslib v1.15, view) and checked by visual inspection.

Growth assays. Most phenotyping was done using liquid growth assays on a BioTek ELx808TM 96-well plate reader. A given strain was grown overnight in YPD at 30° C. and 1.2 ul was then inoculated into 118.8 ul of YPD in the appropriate wells of a 96-well plate. A randomized block design was used to mitigate positional effects. The plates were incubated with shaking at 30° C. or 35° C. on the plate reader and OD600 was acquired every 15 mins until cultures reached the stationary phase. Doubling time value for each culture was calculated using PRECOG (PREsentation and Characterization Of Growth-data) (Fernandez-Ricaud et al., BMC Bioinformatics 17, 249 (2016)). We also performed dilution spot assays. Overnight cultures and their ODs were measured. Cell aliquots were diluted in YPD to an OD of 1. We then performed 10-fold serial dilutions (1:10, 1:100, 1:1,000, 1:10,000). 3 ul of each dilution, including non-diluted overnight culture, were pinned into appropriate plates and incubated at 30° C. or 35° C. Plates were imaged using a BioRAD Gel Doc XR+ Molecular Imager at a standard size of 11.4×8.52 cm2 (width×length) and imaged with epi-illumination using an exposure time of 0.5 s. Images were saved as 600 dpi tiffs.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

What is claimed is:

1. A method for constructing a synthetic chromosome comprising the steps of:
providing host cells comprising an endogenous chromosome;
transforming a cloning vector and a cloning cassette into the host cells;
excising target genomic nucleic acids from the endogenous chromosome;
recombining the excised target genomic nucleic acids with the cloning cassette via homologous recombination to form heterologous vectors comprising cloned sequences;
extracting the heterologous vectors containing the cloned sequences from the host cells;
digesting the heterologous vectors with a restriction endonuclease to release the cloned sequences from the heterologous vectors to provide released cloned sequences; and
introducing the released cloned sequences, a centromere cassette, and a yeast artificial chromosomes or bacterial artificial chromosomes into a second host cell, wherein the released cloned sequences, the centromere cassette, and the yeast artificial chromosomes or bacterial artificial chromosomes recombine with one another via homologous recombination to produce the synthetic chromosome.

2. The method of claim 1, wherein the host cells express a CRISPR endonuclease protein; and wherein the transformation step further comprises transforming into the host cells guide ribonucleic acids (gRNAs) that are complementary to regions flanking the target genomic nucleic acid sequences of the endogenous chromosome, wherein the CRISPR endonuclease protein and the gRNAs excise the target genomic nucleic acid sequences from the endogenous chromosome.

3. The method of claim 1, wherein the cloning cassette comprises two separate linear segments of nucleic acid, wherein a first segment of nucleic acid comprises a first hook region and a second segment of nucleic acid comprises a second hook region, wherein the first hook region comprises formula A-B-C, wherein:
A comprises a unique restriction endonuclease site and first homology region comprising a nucleic acid sequence complementary to first region of the cloning vector;
B comprises a first adapter region comprising a nucleic acid sequence complementary to another adapter region; and
C comprises a first complementary region comprising a nucleic acid sequence complementary to a region flanking the excised target genomic nucleic acids;
the second hook region comprises formula D-E-F, wherein:
D comprises a second complementary region comprising a nucleic acid sequence complementary to a region flanking the excised target genomic nucleic acids;
E comprises a second adapter region comprising a nucleic acid sequence complementary to another adapter region; and
F comprises a unique restriction endonuclease site and a second homology region comprising a nucleic acid sequence complementary to second region of the cloning vector; and
wherein the two separate pieces of nucleic acid are co-transformed into the host cells.

4. The method of claim 1, wherein the cloning vector comprise the cloning cassette, and the cloning cassette comprises a first hook region, a second hook region, and a multiple cloning site (MCS) disposed therebetween, wherein the first hook region comprises formula A-B-C, wherein:
A comprises a unique restriction endonuclease site;
B comprises a first adapter region comprising a nucleic acid sequence complementary to another adapter region; and
C comprises a first complementary region comprising a nucleic acid sequence complementary to a region flanking the excised target genomic nucleic acids;
the second hook region comprises formula D-E-F, wherein:
D comprises a second complementary region comprising a nucleic acid sequence complementary to a region flanking the excised target genomic nucleic acids;
E comprises a second adapter region comprising a nucleic acid sequence complementary to another adapter region; and
F comprises a unique restriction endonuclease site; and
wherein prior to the transformation step, the cloning cassette is linearized by restriction endonuclease digestion using a restriction endonuclease that recognizes and cuts a nucleic acid sequence within the MCS.

5. The method of claim 3, wherein the recombining step further comprises the first complementary region and the second complementary region separately contacting and binding to complementary nucleic acid sequences flanking the excised target genomic nucleic acids; and
the first homology region and the second homology region separately contacting and binding to the first region and second region of the cloning vector, respectively.

6. The method of claim 4, wherein during the digesting step, the restriction endonuclease recognizes and cuts unique restriction endonuclease sites A and F flanking the cloning cassette, thereby releasing the cloned sequences from the heterologous vectors to provide released cloned sequences.

7. The method of claim 5, wherein the released cloned sequences comprise structure B-CS-E, wherein CS comprises the cloned sequences.

8. The method of claim 6, wherein during the introducing step, the first adapter region and the second adapter region of a first released clone sequence are complementary to and binds to one of the first adapter sequence and the second adapter sequence of a second released cloned sequence to permit homologous recombination between the first released clone sequence and the second released clone sequence to produce the synthetic chromosome.

9. The method of claim 3, wherein the cloning cassette is about 100 to about 1000 base pairs in length, wherein each of the B and E moieties is about 25 base pairs to about 200 base pairs in length; each of the C and D moieties is about 50 base pairs to about 200 base pairs in length; and each of the A and F moieties is about 4 base pairs to about 50 base pairs in length.

10. The method of claim 1, wherein the transforming step further comprises introducing a repair template into the host cells, wherein the repair template comprises:
a selectable marker;
a first homology arm; and
a second homology arm;
wherein the first homology arm and the second homology are complementary to nucleic acid sequences of the endogenous chromosome that flank the target genomic nucleic acids to permit homologous recombination between the repair template and the endogenous chromosome, thereby repairing the endogenous chromosome.

11. The method of claim 1, wherein the cloning vectors further comprise a selectable marker, an origin of replication, a centromere sequence, a sop genes, an oriV sequence, an oriS sequence, a rep gene, or a combination thereof.

12. The method of claim 1, wherein the centromere cassette comprises an origin of replication, a centromere sequence, and an adapter arm flanking both the origin of replication and the centromere sequence, and optionally, a selectable marker; wherein the adapter arm is complementary to another adapter region of the cloning cassette.

13. The method of claim 1, wherein the yeast artificial chromosomes or bacterial artificial chromosomes comprises a selectable marker, and homology arms that are complementary to adapter regions of the cloning cassette.

14. The method of claim 1, wherein the synthetic chromosome comprises deletion of segments of nucleic acid as compared to the endogenous chromosome; or
wherein the synthetic chromosome comprises a rearrangement of nucleic acid segments as compared to an arrangement of corresponding nucleic acid segments of the endogenous chromosome.

15. The method of claim 1, wherein the host cells are cells of at least two different genus or species, wherein the synthetic chromosome is a chimeric chromosome comprising nucleic acid sequences from the at least two different genus or species.

16. The method of claim 1, wherein the host cells are one or more of a human cell, a fungal cell, or a bacterial cell.

17. A recombinant cell having at least one synthetic chromosome produced according to the method of claim 1.
